(19) United States
(12) Patent Application Publication
(10) Pub. No.: US 2026/0258120 A1
(43) Pub. Date: Sep. 3, 2026

(54) CHIMERIC ANTIGEN RECEPTORS FOR DIRECT AND INDIRECT TARGETING OF FIBRONECTIN-POSITIVE TUMORS

(71) Applicant: St. Jude Children's Research Hospital, Inc., Memphis, TN (US)

(72) Inventors: Stephen Gottschalk, Memphis, TN (US); Jessica Wagner, Memphis, TN (US); Timothy Isham Shaw, Memphis, TN (US); Jinghui Zhang, Memphis, TN (US)

(73) Assignee: St. Jude Children's Research Hospital, Inc., Memphis, TN (US)

(21) Appl. No.: 19/468,667

(22) Filed: Feb. 3, 2026

Related U.S. Application Data
(63) Continuation of application No. 17/628,483, filed on Jul. 17, 2020, now Pat. No. 12,570,730.
(60) Provisional application No. 62/876,158, filed on Jul. 19, 2019.

(51) Int. Cl. C07K 16/18; A61K 40/11; A61K 40/31; A61K 40/42; A61P 35/00; C07K 14/705

(52) U.S. Cl. CPC C07K 16/18; A61K 40/11; A61K 40/31; A61K 40/4202; A61P 35/00; C07K 14/70517; C07K 14/70521; A61K 2239/31; A61K 2239/38; A61K 2239/55; C07K 2317/622; C07K 2319/03; C07K 2319/33

(57) ABSTRACT

The application relates to a chimeric antigen receptor that directly and/or indirectly targets cells and their uses in tumor immunotherapy. The application also relates to polynucleotides that encode the chimeric antigen receptor and optionally accessory genes, vectors, and host cells comprising the chimeric antigen receptor and optionally a second antigen targeting moiety (e.g., a second chimeric antigen receptor or a bispecific molecule). The application also relates to methods for preparing host cells comprising the chimeric antigen receptor and optionally the second antigen targeting moiety.

### CROSS-REFERENCE TO RELATED APPLICATIONS

This application is a continuation of U.S. patent application Ser. No. 17/628,483, filed Jan. 19, 2022, which is a National Stage Application of International Application No. PCT/US2020/042565, filed Jul. 17, 2020 which claims priority to U.S. Provisional Application No. 62/876,158, filed Jul. 19, 2019, all of which are herein incorporated by reference in their entireties.

### SEQUENCE LISTING

The instant application contains a Sequence Listing which has been submitted electronically in XML file format and is hereby incorporated by reference in its entirety. Said XML copy, created on Mar. 19, 2026, is named 243734_000237_SL.xml and is 236,127 bytes in size.

### FIELD

The application relates to chimeric antigen receptors (CARs), particularly CARs with improved antitumor properties (e.g., CARs the directly and/or indirectly target cells), and their uses in tumor immunotherapy (e.g., adoptive cell therapy). The application further relates to methods for activating T-cells and other lymphocytes resulting in an immune response against a target antigen. The application further relates to therapeutic cells that express said CARs and methods for treating patients using the modified therapeutic cells.

### BACKGROUND

The outcome of recurrent/refractory solid tumor and brain tumors remains poor despite aggressive multimodal therapy. Cell therapy with genetically modified T-cells expressing chimeric antigen receptors (CARs) hold the promise to improve outcomes since they kill tumor cells through mechanisms that are distinct from conventional therapies such a chemotherapy and radiation. While CAR T-cell therapy has been successful for hematological malignancies, leading to the FDA approval of two CD19-CAR T-cell products, the antitumor activity of CAR T-cells for solid tumors and brain tumors have been limited.⁵⁻⁸ For example, in a Phase I clinical study in which 16 patients with osteosarcoma were infused with up to 1×10⁸/m² HER2-CAR T-cells.⁵ HER2-CAR T-cells trafficked to tumor sites and persisted at low levels for >6 weeks without evident toxicities. Four patients had stable disease for up to 14 months; of these, three are alive with no evidence of disease after tumor removal with median follow up of three to five years.

The lack of efficacy of CAR T-cell therapy for solid tumors is most likely multifactorial, including heterogeneous expression of a limited array of tumor associated antigens (TAAs), loss of TAAs following natural or therapy-induced selective pressure, limited homing of T-cells to solid tumors, concerns about on-target/off target toxicity, and the hostile tumor micro-environment (TM).⁹⁻¹¹ In addition, nonmalignant cells present in the tumor stroma that support tumor growth such as vascular endothelial cells and cancer associated fibroblasts are not targeted by standard CAR T-cells.

While adult solid tumors and brain tumors express unique neoantigens, they use is problematic since most are private and rarely shared between patients, making it almost impossible to generate a CAR T-cell product that could be used for multiple patients.⁵⁸ In addition, pediatric solid tumors and brain tumors carry a low neoantigen load, which precludes its targeting with neoantigen-specific CARs. To increase CAR T-cell specificity when targeting solid and brain tumors, the unique extracellular matrix (ECM) within the tumor micro-environment (TME) may serve as a target as it is an environment that is distinct to tumors alone.

Fibronectin is a major component of the ECM. Tumor cells as well as tumor endothelial cell express a unique variant of fibronectin called FN-EDB, EDB, or EIIIB. Several monoclonal antibodies have been developed for targeting and imaging of EDB+ positive tumors ²⁷⁻²⁹

### SUMMARY OF THE INVENTION

As specified in the Background section above, there is a great need in the art for CARS that target new antigens in solid tumors and brain tumors. EDB is an ideal target, since it is a major component of the tumor's ECM that is produced by tumor cells as well as endothelial cells of the tumor neovasculature. The present application addresses these and other needs.

In one aspect provided herein is a polynucleotide encoding a first chimeric antigen receptor (CAR) comprising: (a) an extracellular target-binding domain comprising a Fibronectin Extradomain B (FN-EDB)-binding moiety, (b) a hinge domain, (c) a transmembrane domain, and (d) a cytoplasmic domain comprising (i) optionally one or more costimulatory domains, (ii) a signaling domain.

In some embodiments, the FN-EDB-binding moiety is an anti-FN-EDB single chain variable fragment (scFv). In some embodiments, the anti-FN-EDB scFv is derived from antibody L19 (L19 scFv). In some embodiments, the L19 scFv comprises the amino acid sequence set forth in SEQ ID NO: 25, or an amino acid sequence having at least 80% identity thereof. In some embodiments, the nucleotide sequence encoding the L19 scFV comprises the sequence set forth in SEQ ID NO: 26, or a nucleotide sequence having at least 80% identity thereof.

In some embodiments, the extracellular target-binding domain further comprises a leader sequence. In some embodiments, the leader sequence comprises the amino acid sequence set forth in SEQ ID NO: 61, or an amino acid sequence having at least 80% identity thereof. In some embodiments, the nucleotide sequence encoding the leader sequence comprises the sequence set forth in SEQ ID NO: 62, or a nucleotide sequence having at least 80% identity thereof.

In some embodiments, the hinge domain is derived from IgG1, CD8a stalk, or CD28.

In some embodiments, the hinge domain comprises the amino acid sequence set forth in SEQ ID NO: 91, or an amino acid sequence having at least 80% identity thereof. In some embodiments, the nucleotide sequence encoding the hinge domain comprises the sequence set forth in SEQ ID NO: 92, or a nucleotide sequence having at least 80% identity thereof.

In some embodiments, the transmembrane domain is derived from CD28, CD8α, CD4, or CD3ζ.

In some embodiments, the transmembrane domain comprises the amino acid sequence set forth in SEQ ID NO: 36, or an amino acid sequence having at least 80% identity thereof. In some embodiments, the nucleotide sequence encoding the transmembrane domain comprises the sequence set forth in SEQ ID NO: 37, or a nucleotide sequence having at least 80% identity thereof.

In some embodiments, the costimulatory domain is derived from CD28, CD27, CD40, CD134, CD137, CD226, CD79A, ICOS, or MyD88.

In some embodiments, the costimulatory domain comprises the amino acid sequence set forth SEQ ID NO: 40, or an amino acid sequence having at least 80% identity thereof. In some embodiments, the nucleotide sequence encoding the costimulatory domain comprises the sequence set forth in SEQ ID NO: 41, or a nucleotide sequence having at least 80% identity thereof.

In some embodiments, the lymphocyte activation domain is derived from CD3ζ, DAP10, DAP12, Fc epsilon receptor I γ chain (FCER1G), CD3δ, CD3ε, CD3γ, CD226, or CD79A.

In some embodiments, the CD35 signaling domain comprises the amino acid sequence set forth in SEQ ID NO: 57, or an amino acid sequence having at least 80% identity thereof. In some embodiments, the nucleotide sequence encoding the signaling domain comprises the sequence set forth in SEQ ID NO: 58, or a nucleotide sequence having at least 80% identity thereof.

In some embodiments, the CAR encoded by the polynucleotide comprises the amino acid sequence as set forth in SEQ ID NO: 95, or an amino acid sequence having at least 80% identity thereof. In some embodiments, the polynucleotide encoding the CAR comprises the nucleotide sequence as set forth in SEQ ID NO: 2, 96 or 120, or a nucleotide sequence having at least 80% identity thereof.

In some embodiments, the polynucleotide comprises at least one additional polynucleotide sequence encoding a second gene.

In some embodiments, the second gene encodes a truncated CD19 (tCD19) polypeptide. In some embodiments, the tCD19 comprises the amino acid sequence set forth in SEQ ID NO: 65, or an amino acid sequence having at least 80% identity thereof. In some embodiments, the nucleotide sequence encoding the tCD19 comprises the sequence set forth in SEQ ID NO: 66, or a nucleotide sequence having at least 80% identity thereof.

In some embodiments, the second gene encodes a synthetic notch (synNotch) receptor. In some embodiments, the synNotch receptor comprises the amino acid sequence set forth in SEQ ID NO: 67, or an amino acid sequence having at least 80% identity thereof. In some embodiments, the nucleotide sequence encoding the synNotch receptor comprises the sequence set forth in SEQ ID NO: 68, or a nucleotide sequence having at least 80% identity thereof.

In some embodiments, the polynucleotide further comprises a polynucleotide that encodes an additional antigen binding moiety. In some embodiments, the antigen-binding moiety is an anti-FN-EDB binding moiety. In some embodiments, the FN-EDB-binding moiety is an anti-FN-EDB single chain variable fragment (scFv). In some embodiments, the anti-FN-EDB scFv is derived from antibody L19 (L19 scFv). In some embodiments, the L19 scFv comprises the amino acid sequence set forth in SEQ ID NO: 25, or an amino acid sequence having at least 80% identity thereof. In some embodiments, the nucleotide sequence encoding the L19 scFV comprises the sequence set forth in SEQ ID NO: 26, or a nucleotide sequence having at least 80% identity thereof.

In some embodiments, the at least one additional polynucleotide sequence is operably linked to the sequence encoding a CAR via a sequence encoding a self-cleaving peptide and/or an internal ribosomal entry site (IRES).

In some embodiments, the self-cleaving peptide is a 2A peptide. In some embodiments, the 2A peptide is T2A, P2A, E2A, or F2A peptide. In some embodiments, the self-cleaving 2A peptide encoded comprises the amino acid sequence set forth in SEQ ID NO: 69, or an amino acid sequence having at least 80% identity thereof. In some embodiments, the sequence encoding the self-cleaving 2A peptide comprises the nucleotide sequence set forth in SEQ ID NO: 70, or a nucleotide sequence having at least 80% identity thereof.

In one aspect provided herein is a polynucleotide encoding a synthetic notch (synNotch) receptor operably linked to at an antigen targeting moiety. In certain embodiments, the antigen targeting moiety can be, but is not limited to, a chimeric antigen receptors (CAR), bispecific T-cell engager (BiTE), or an antibody. In certain embodiments, the synNotch receptor is operably linked to the antigen targeting moiety via a sequence encoding a self-cleaving peptide and/or an internal ribosomal entry site (IRES). In some embodiments, the synNotch receptor comprises the amino acid sequence set forth in SEQ ID NO: 67, or an amino acid sequence having at least 80% identity thereof. In some embodiments, the nucleotide sequence encoding the synNotch receptor comprises the sequence set forth in SEQ ID NO: 68, or a nucleotide sequence having at least 80% identity thereof.

In some embodiments, the polynucleotide is a DNA molecule. In other embodiments, the polynucleotide is an RNA molecule.

In another aspect provided herein is a recombinant vector comprising a polynucleotide described herein. In some embodiments, the vector is a viral vector. In some embodiments, the viral vector is a retroviral vector, a lentiviral vector, an adenoviral vector, an adeno-associated virus vector, an alphaviral vector, a herpes virus vector, or a vaccinia virus vector. In some embodiments, the viral vector is a retroviral vector. In some embodiments, the vector is a non-viral vector.

In another aspect provided herein is a chimeric antigen receptor (CAR) encoded by a polynucleotide described herein.

In another aspect provided herein is an isolated host cell comprising a polynucleotide described herein or a recombinant vector described herein. In some embodiments, host cell comprises a CAR as described herein.

In some embodiments, the host cell is an immune cell. In some embodiments, the immune cell is a T-cell, a NK cell, or a macrophage. In some embodiments, the T-cell is selected from a CD8+ T-cell, a CD4+ T-cell, a cytotoxic T-cell, an αβ T-cell receptor (TCR) T-cell, an invariant natural killer T (iNKT) cell, a γδ T-cell, a memory T-cell including memory stem T-cell (TSCM), a naïve T-cell, an effector T-cell, a T-helper cell, and a regulatory T-cell (Treg).

In some embodiments, the host cell further comprises a second CAR or a bispecific molecule and wherein the expression of the second CAR or the bispecific molecule is induced by the activation of the first CAR. In some embodiments, the expression of the second CAR or bispecific molecule is induced using a synthetic Notch (synNotch) receptor. In some embodiments, the expression of the second CAR or bispecific molecule is induced using at least one nuclear factor of activated T-cells (NFAT) dependent-promoter.

In some embodiments, the bispecific molecule is a bispecific T-cell engager (BiTE) or a bispecific antibody.

In some embodiments, the second CAR or the bispecific molecule targets a tumor associated antigen.

In some embodiments, the tumor associated antigen is 5T4, αβ6 integrin, B7-H3, B7-H6, CAIX, CD19, CD20, CD22, CD30, CD33, CD44, CD44v6, CD44v7/8, CD70, CD123, CD138, CD171, CEA, Claudin-6 or 18, CSPG4, EGFR, EGFR family including ErbB2 (HER2), EGFRvIII, EGP2, EGP40, EPCAM, EphA2, EpCAM, FAP, fetal AchR, FRα, GD2, GD3, MAGE1, NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, Survivin, TAG72, TEMs, or VEGFR2.

In some embodiments, the host cell has been activated and/or expanded ex vivo. In some embodiments, wherein the host cell is an allogeneic cell. In some embodiments, the host cell is an autologous cell.

In some embodiments, the host cell is isolated from a subject having a tumor, wherein one or more cells of the tumor express FN-EDB. In some embodiments, the tumor is a solid tumor. In some embodiments, the tumor is selected from breast cancer, prostate cancer, bladder cancer, oral squamous cell carcinoma, head and neck squamous cell carcinoma, colorectal cancer, lung cancer, brain tumors, melanoma, bone, pediatric solid tumors and brain tumors, and lymphoma.

In some embodiments, the host cell is derived from a blood, marrow, tissue, or a tumor sample.

In another aspect provided herein is a pharmaceutical composition comprising a host cell described herein and a pharmaceutically acceptable carrier and/or excipient.

In another aspect provided herein is a method of generating an isolated host cell described herein, said method comprising genetically modifying the host cell with a polynucleotide described herein or a recombinant vector described herein.

In some embodiments, the method comprises a vector. In some embodiments, the vector is a viral vector and the genetic modification is conducted by a transduction using said vector.

In some embodiments, the method comprises a genetic modification conducted ex vivo.

In some embodiments, the method further comprises activation and/or expansion of the host cell ex vivo before, after and/or during said genetic modification.

In another aspect provided herein is a method for killing a tumor cell expressing FN-EDB, said method comprising contacting the cell with a host cell described herein or a pharmaceutical composition described herein.

In another aspect provided herein is a method for indirectly killing a tumor cell expressing a tumor associated antigen, said method comprising contacting said cell with a host cell described herein or a pharmaceutical composition described herein.

In another aspect provided herein is a method for treating a tumor in a subject in need thereof, wherein one or more cells of the tumor express FN-EDB, said method comprising administering to the subject a therapeutically effective amount of host cells described herein or a pharmaceutical composition described herein.

In another aspect provided herein is a method for treating a tumor and/or inhibiting tumor neovascularization in a subject in need thereof, wherein one or more cells of the tumor or non-tumor cells within the tumor micro-environment express FN-EDB and the tumor cell expresses a tumor associated antigen, said method comprising administering to the subject a therapeutically effective amount of host cells described herein or a pharmaceutical composition described herein.

In some embodiments, the non-tumor cells within the tumor micro-environment are vascular endothelial cells and/or tumor stroma. In some embodiments, the tumor is a solid tumor. In some embodiments, the tumor is selected from breast cancer, prostate cancer, bladder cancer, oral squamous cell carcinoma, head and neck squamous cell carcinoma, colorectal cancer, lung cancer, brain tumors, melanoma, bone, pediatric solid tumors and brain tumors, and lymphoma.

In another aspect provided herein is a method comprising: a) isolating T-cells, NK cells, iNKT cells or macrophages from the subject or generating T-cells, NK cells, iNKT cells or macrophages from stem cells including induced pluripotent stem cells (IPS cells); b) genetically modifying said T-cells, NK cells, iNKT cells, macrophages or stem cells including IPS cells ex vivo with a polynucleotide described herein or a vector described herein; c) optionally, expanding and/or activating said T-cells, NK cells, iNKT cells or macrophages before, after or during step b); and d) introducing the genetically modified T-cells, NK cells, iNKT cells or macrophages into the subject.

In some embodiments, the subject is human. In some embodiments, the subject is an adult. In some embodiments, the subject is a child.

These and other aspects of the present invention will be apparent to those of ordinary skill in the art in the following description, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show schematics of direct or indirect targeting of tumor cells with EDB-CAR T-cells. While FN-EDB is a secreted protein, it plays a major role in cellular adhesion, allowing EDB-CAR T-cells to directly recognize EDB-producing tumor cells (Direct targeting; FIG. 1A). As EDB is secreted, it is also an ideal target for indirect targeting of tumor cells, an approach in which T-cells are genetically modified to express an EDB-specific SynNotch (EDB-SynNotch) receptor. Once EDB-SynNotch T-cells encounter EDB within the extracellular matrix (ECM; FIG. 1B), the EDB-SynNotch receptor induces the expression of a tumor associated antigen (TAA)-specific CAR or bispecific T-cell engage (BiTE).

FIG. 2 shows expression of FN-EDB in pediatric solid tumors and brain tumors including sarcoma using the Pediatric Cancer Genome Project (PCGP) dataset. Fragments Per Kilobase Million (FPKM) and t-Distributed Stochastic Neighbor Embedding (t-SNE) plots show expression in pediatric solid tumors and brain tumors.

FIGS. 3A-3C demonstrates that EDB-CAR T-cells recognize and kill FN-EDB+ tumor cells. A retroviral vector was designed encoding an EDB-specific CAR (EDB-CAR) using the EDB-specific L19 scFv, a short IgG 1 hinge, a CD28 transmembrane domain and a CD28.3 signaling domain; in addition, the retroviral vector encoded a 2A sequence and truncated CD19 (FIG. 3A). FACS analysis of CAR expression on transduced T-cells (FIG. 3B). Only EDB-CAR T-cells secrete IFNγ in the presence of recombinant (rh) FN-EDB; non-transduced (NT) T-cells do not (n=3) (FIG. 3C).

FIGS. 4A-4D demonstrate that EDB-CAR T-cells recognize and kill EDB-positive tumor cells. Tumor cells (LM7, A549, U87) express EDB in contrast to normal fibroblasts (FIG. 4A). EDB-CAR T-cells (FIG. 4B) secrete IFNγ and kill EDB+ tumor cells in contrast to NT T-cells (FIG. 4C) (n=3). EDB-CAR T cells do not kill EDB-fibroblasts in contrast to EDB+A549 tumor cells (FIG. 4D).

FIGS. 5A-5D demonstrate that EDB-CAR T-cells have antitumor activity in the A549 subcutaneous tumor model in vivo. To determine if EDB-CAR T-cells have antitumor activity in vivo, 2×10⁶ A549 cells were injected s.c. into immunodeficient NSG mice. On day 20, mice received a single iv of 3×10⁵ T-cells (FIG. 5A). Mice received either NT, EphA2-CAR, or EDB-CAR T-cells. Tumor growth was followed by caliper measurements (FIG. 5B). Tumors were stained for CD31 (FIG. 5C; representative images). Vessel density was quantified (n=4 tumors; dots represent average of 3 blind scores per tumor; 2 fields of view per tumor) (FIG. 5D).

FIGS. 6A-6C demonstrate that EDB-CAR T-cells have antitumor activity in the U87 subcutaneous tumor model in vivo; 2×10⁶ U87 cells were injected subcutaneously, and on day 7, mice received a single intravenous injection of 1×10⁶ EDB-CAR or NT T-cells (n=10 per group) (FIG. 6A). Tumor growth was measured by serial caliper measurements. EDB-CAR T-cells had potent antitumor activity (FIG. 6B) that translated into a significant survival advantage (FIG. 6C).

FIGS. 7A-7B demonstrate that EDB-CAR T-cells have antitumor activity in systemic tumor model in vivo. 2×10⁶ GFP-ffluc-expressing A549 cells were injected i.v. into immunodeficient NSG mice, and on day 7 mice received i.v. 1×10⁶ NT or EDB-CAR T-cells (FIG. 7A). Tumor growth was followed by bioluminescence imaging (FIG. 7B).

FIGS. 8A-8C demonstrate utilization of the synNotch system. FIG. 8A depicts a scheme of a vector that encodes an EDB-synNotch receptor, which will induce the expression of a TAA-specific CAR or BiTE molecule. FIG. 8B depicts how EDB-synNotch receptor induce the expression of a TAA-specific CAR or BiTE molecule. FIG. 8C depicts a vector for combined indirect and direct targeting of tumor cells encoding an EDB-CAR T-cells, an EDB-SynNotch receptor, and an inducible gene encoding for a TAA-specific CAR or BiTE molecule. DBD-DNA binding domain. MND-a promoter that comprising the modified LTR U3 region of MoMuLV (Moloney Murine Leukemia Virus), in which the enhancer sequence is replaced by myeloproliferative sarcoma virus enhancer and the negative control region is deleted. VP-viral protein sequence.

FIGS. 9A-9B present additional EDB-CARs.

FIGS. 10A-10LL present exemplary sequences of the disclosure.

### DETAILED DESCRIPTION

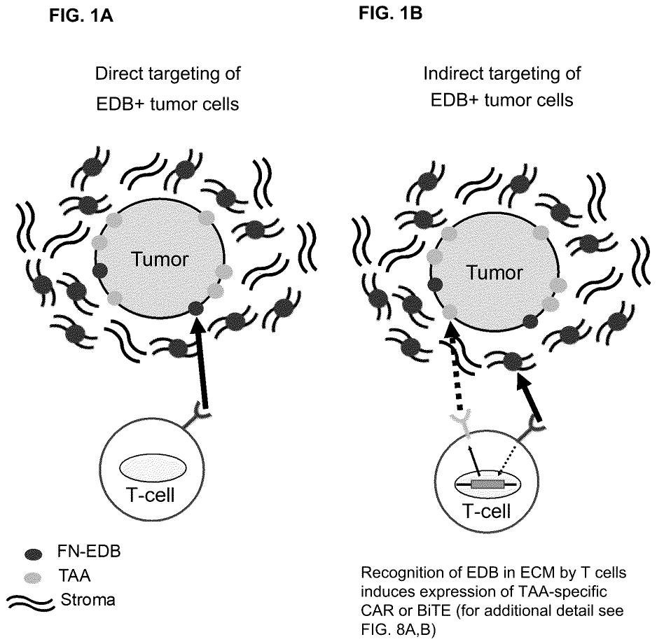

The present disclosure provides chimeric antigen receptors (CARs) and T-cells and other lymphocytes expressing said CARs that target antigens located on the target tumor cell and/or the extracellular matrix (ECM) within the tumor micro-environment (TME) (e.g., targeting vascular endothelial cells and/or tumor stroma) with special focus on fibronectin-EDB (oncofetal fibronectin, FN-EDB, EDB, EIIIB). In certain embodiments, the CAR T-cells directly target tumor cells (see e.g., FIG. 1A; i.e., EDB-specific CAR T-cells recognize an antigen that is also present on the cell surface of tumor cells and kill it). In certain embodiments, the EDB-specific T-cells indirectly target tumor cells (see e.g., FIG. 1B; i.e., synthetic Notch (synNotch)-receptor T-cells recognize an antigen that is present in the tumor ECM). Upon recognition, the synNotch receptor induces the expression of a second molecule (e.g., a second transgene) within the T-cells or other lymphocytes that enables tumor cell killing. By way of example and not limitation, the second molecule includes a tumor-specific CAR, bispecific T-cell engager (BiTE), dual affinity retargeting (DART) antibody, or bispecific antibodies that redirect other immune cells (for example, but not limited to macrophages, NK cells) to kill tumor cells.

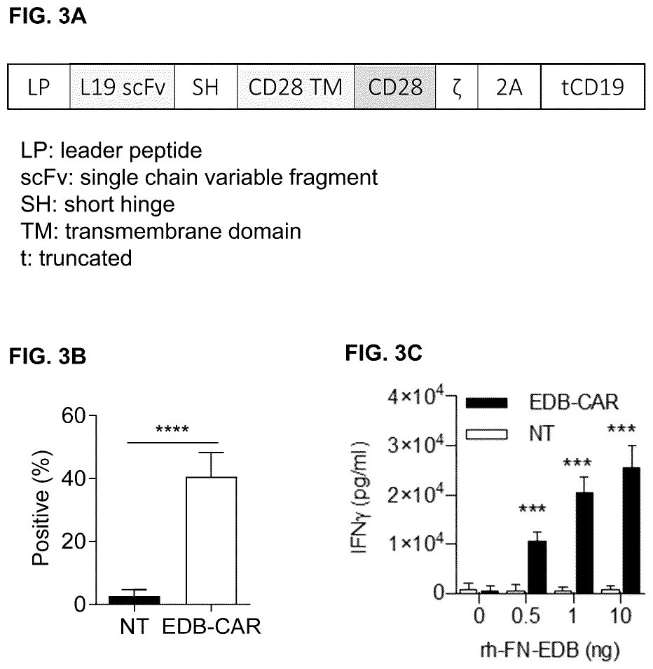

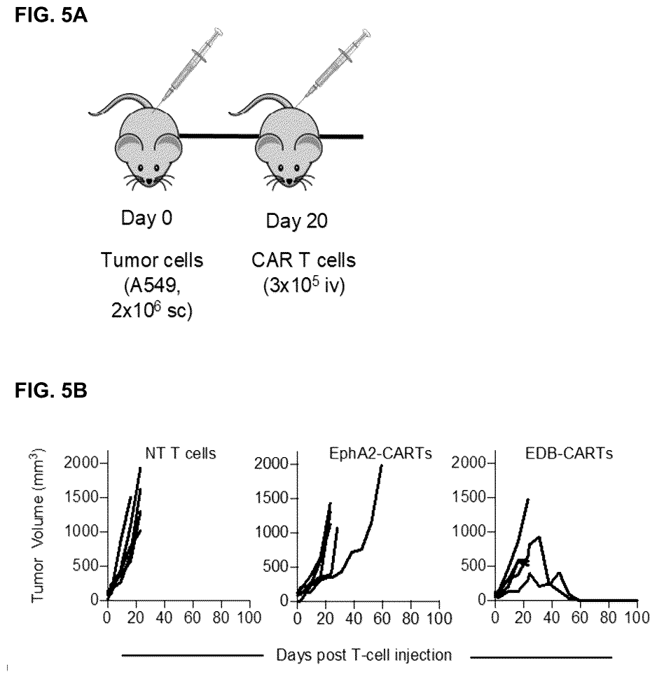

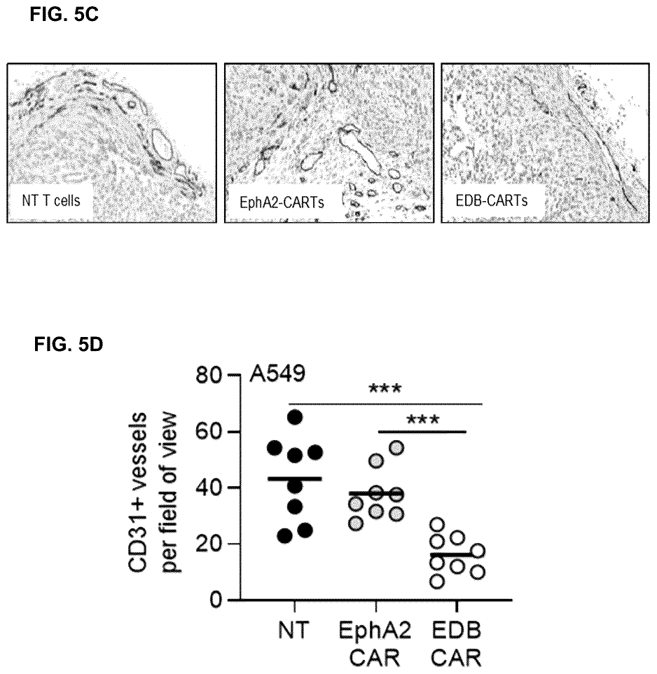

The TME contains the ECM, which is formed through the secretion of distinct proteins while the tumors develop. This ECM possesses molecules that are tumor specific. One example of such an ECM molecule is fibronectin-EDB (oncofetal fibronectin, FN-EDB, EDB, EIIIB), which is a splice variant of fibronectin and a major component of the ECM in solid tumors. EDB expression is limited to fetal cells, tumor cells, endothelial of the tumor neovasculature and not found in either the plasma or normal healthy tissue in adults.^{14,22} Further, EDB is homologous across multiple species including mouse, rabbit, and human.^{23,24} These tumor-specific attributes and the shared homology make EDB a promising target for both pre-clinical and clinical studies to reduce off-target toxicity in patients that can be caused by widespread cytokine release leading to potential organ failure. A high affinity EDB-specific monoclonal antibody (MAb), L19, was successfully tested in 40 patients demonstrating localization to the tumor site.²⁷⁻²⁹ While EDB is secreted, Example 1 demonstrates that it is arrayed on the cell surface since EDB-CAR T-cells have antitumor activity in preclinical in vitro and in vivo (FIGS. 3 to 8). In addition, the Example 3 demonstrates that EDB-CAR T-cells target the tumor vasculature (FIG. 5). Other examples of splice variants within the ECM that could be targeted with this approach include the EDA domain of fibronectin, and splice variants of other proteins, but not limited to, tenascin-C, -N and R, SPARC (Secreted Protein Acidic And Cysteine Rich), and the CCN (Cellular Communication Network) family (see e.g., Yoshida et al., Cell Adh Migr. 2015, 9 (1-2): 96-104) and Viloria and Hill, Biomol Concepts, 2016, 7 (2): 117-32; both of which are incorporated herein by reference in their entirety for all purposes).

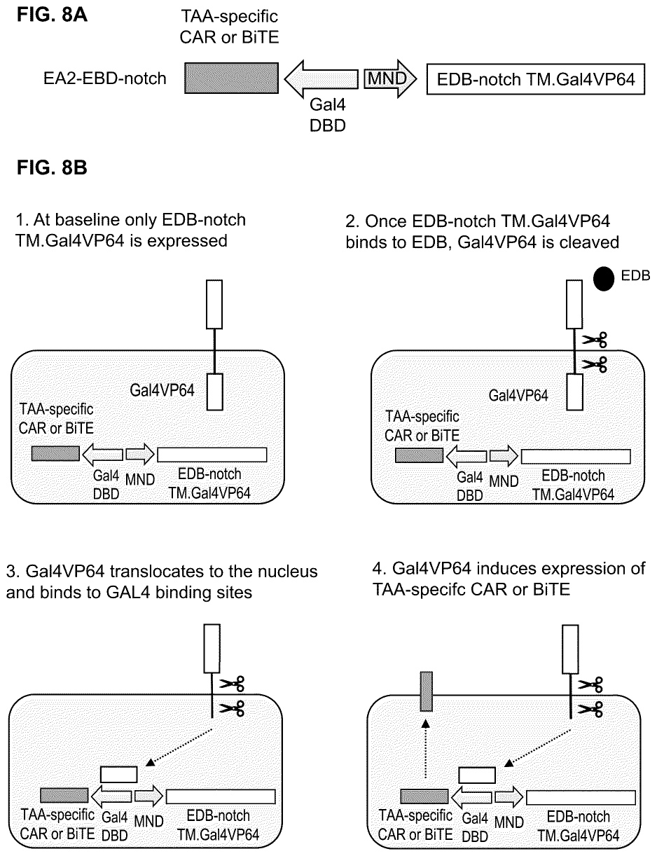

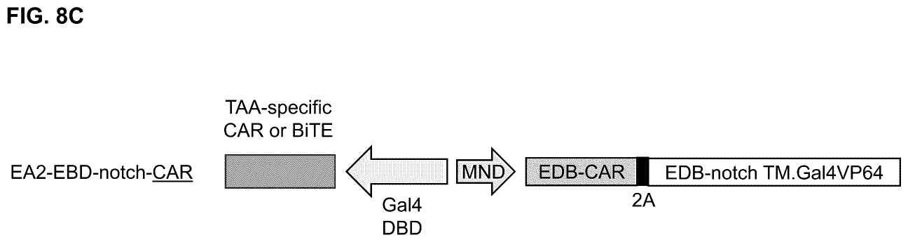

Targeting a single tumor associated antigen (TAA) carries the risk of selecting immune escape mutants, which no longer express the targeted TAA, 11,36,47 and/or in some instances can cause on-target/off target toxicity. Both limitations can be addressed with the approaches described herein. For example, in certain aspects of the present disclosure, EDB-SynNotch (e.g., SEQ ID NO: 67, 93, or 121) expressed in T-cells or other lymphocytes can be used to induce the expression of a second antigen targeting molecule (e.g., a second CAR or the bispecific molecule (e.g., bispecific T-cell engager (BITE) or bispecific antibody)) that targets a TAA expressed on the target tumor cell (FIG. 8A,B).

This allows for targeting TAA that are potentially expressed in normal tissues since the TAA-specific molecule is only expressed by the T-cells in the EDB+ tumor microenvironment. The selection of immune escape variants can be prevented by combining the EDB-CAR and EDB-SynNotch receptor approach and a representative vector for this combined approach is shown in FIG. 8C). Thus, advantageously, only the CAR T-cells activated by the antigen present on the surrounding stroma will be induced to express the TAA targeting construct.

The synNotch receptor system is ideal to couple T-cell or other lymphocyte activation to expression of a protein since in contrast to other activating T-cell or other lymphocyte promoters, synNotch receptors have no baseline activity as they do not share any common signaling intermediates, making them an attractive option for the current approach. SynNotch receptors contain its core regulatory domain from the cell-cell signaling receptor Notch, but it further comprises a synthetic extracellular recognition domain (e.g., EDB scFv) and a synthetic transcriptional domain that will induce the expression of a desired target gene (e.g., second CAR or bispecific molecule (e.g., BiTE or bispecific antibody)). When the synNotch receptor engages with its cognate antigen (e.g., EDB), the synNotch receptor undertakes induced transmembrane cleavage that releases the intracellular transcriptional domain to enter the nucleus and induces the expression of the target gene via an upstream promoter (FIG. 8B).

Alternatively, the expression of the second antigen targeting molecule can be under the control of at least one nuclear factor of activated T-cells (NFAT) dependent-promoter, which results in the expression of the second antigen targeting molecule is tightly linked to T-cell activation (e.g., but not limited to Th1 and Th2 cytokines or cell surface molecules (e.g., PD-1, CD137)).

In certain embodiments, the present disclosure provides CARs and T-cells or other lymphocytes expressing said CARs that target FN-EDB located on the target tumor cell (see FIG. 1A) and/or the ECM within the TME. In certain embodiments, the CARs that target FN-EDB on the ECM enable the activation of CAR T-cells or other lymphocytes within the tumors that are not in direct contact with tumor cells (see FIG. 1B). The activation of the CAR T-cells or other lymphocytes induces the expression of a second antigen targeting molecule (e.g., a second CAR or a bispecific molecule (e.g., BiTE or bispecific antibody)) that targets a tumor associated antigen expressed on the target tumor cell. At that point, the CAR T-cell or other lymphocyte will then become in direct contact with the target tumor cell to induce death of the tumor cell or secrete a molecule (e.g. BiTE or bispecific antibody) that will induce tumor cell killing.

In certain embodiments, a tumor associated antigen can be, but not limited to, 5T4, avB6 integrin, B7-H3, B7-H6, CAIX, CD19, CD20, CD22, CD30, CD33, CD44, CD44v6, CD44v7/8, CD70, CD123, CD138, CD171, CEA, Claudin-6 or 18, CSPG4, EGFR, EGFR family including ErbB2 (HER2), EGFRVIII, EGP2, EGP40, EPCAM, EphA2, EpCAM, FAP, fetal AchR, FRα, GD2, GD3, MAGE1, NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, Survivin, TAG72, TEMs, or VEGFR2.

CARs are primarily comprised of 1) an antigen-binding moiety, such as but not limited to a single-chain variable fragment (scFv) derived from an antigen-specific monoclonal antibody, and 2) a lymphocyte activation domain, such as but not limited to the ζ-chain from the T-cell receptor CD3. These two regions are fused together via a transmembrane domain. A hinge domain is usually required to provide more flexibility and accessibility between the antigen-binding moiety and the transmembrane domain. Upon transduction, the lymphocyte expresses the CAR on its surface, and upon contact and ligation with the target antigen, it signals through the lymphocyte activation domain (e.g., CD3ζ chain) inducing cytotoxicity and cellular activation.

CAR constructs may also include co-stimulatory polypeptides to boost the CAR-induced immune response. The most commonly used co-stimulating molecules include CD28 and 4-1BB, which promotes both T-cell proliferation and cell survival. Another example of co-stimulatory domains is a MyD88/CD40 molecule that can be used with or without the use of a separate dimerization agent. Additional CAR constructs may also include three signaling domains (e.g., CD3 ζ, CD28, and 4-1BB), which further improves lymphocyte cell survival and efficacy.

In certain embodiments, the polynucleotide encoding the CAR is further operably linked to a second gene. In certain embodiments, the second gene encodes a truncated CD19 (tCD19) polypeptide. In certain embodiments, the second gene encodes a synthetic notch (synNotch) receptor.

### Definitions

The term “chimeric antigen receptor” or “CAR” as used herein is defined as a cell-surface receptor comprising an extracellular target-binding domain, a transmembrane domain and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present disclosure are intended primarily for use with lymphocyte such as T-cells and natural killer (NK) cells.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+CD8+ T-cell, or any other subset of T-cells. Other illustrative populations of T-cells suitable for use in particular embodiments include naive T-cells and memory T-cells. Also included are “NKT cells”, which refer to a specialized population of T-cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated α- and β-TCR chains, the TCR in γδ T-cells is made up of a γ-chain and a δ-chain. γδ T-cells can play a role in immunosurveillance and immunoregulation, and were found to be an important source of IL-17 and to induce robust CD8+ cytotoxic T-cell response. Also included are “regulatory T-cells” or “Tregs” refers to T-cells that suppress an abnormal or excessive immune response and play a role in immune tolerance. Tregs cells are typically transcription factor Foxp3-positive CD4+ T cells and can also include transcription factor Foxp3-negative regulatory T-cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′) 2, and Fv fragments; polypeptides derived from T-cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include T-cells and natural killer cells that contain the DNA or RNA sequences encoding the CAR and express the CAR on the cell surface. Host cells may be used for enhancing T-cell activity, natural killer cell activity, treatment of tumors, and treatment of autoimmune disease.

The terms “activation” or “stimulation” means to induce a change in their biologic state by which the cells (e.g., T-cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T-cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” or “variant” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative or variant of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative or variant of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative or variant of; (d) a polypeptide encoded by nucleic acids can hybridize under high, moderate or typical stringency hybridization conditions to nucleic acids encoding the polypeptide it is a derivative or variant of; (e) a polypeptide encoded by a nucleotide sequence that can hybridize under high, moderate or typical stringency hybridization conditions to a nucleotide sequence encoding a fragment of the polypeptide, it is a derivative or variant of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative or variant of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T-cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors

In certain aspects, the disclosure provides a CAR that targets antigens located on the target tumor cell and/or the ECM within the TME to allow for direct targeting of the tumor cell and/or ECM (e.g., neovasculature, stromal cells such as cancer associated fibroblasts, etc.).

In other aspects, the disclosure provides a CAR that targets antigens located on the ECM within the TME to allow for indirect targeting of the tumor cell. In particular, the CAR is co-expressed with a second antigen targeting molecule (e.g., a second CAR or a bispecific molecule (e.g., a BiTE or bispecific antibody)) that targets a TAA expressed on the target tumor cell. In certain embodiments, activation of the lymphocyte expressing the CAR induces the expression of the second antigen targeting molecule. In certain embodiments, the expression of the second antigen targeting molecule is induced by a second molecule. In certain embodiments, the second molecule can be a synNotch receptor. In certain embodiments, the synNotch receptor is operably linked to the polynucleotide encoding the CAR. In some embodiments, the second molecule can be an NFAT that is activated by the activation of the T-cell or other lymphocyte.

In certain aspects, the present disclosure provides a polynucleotide encoding a first chimeric antigen receptor (CAR) comprising: (a) an extracellular target-binding domain, (b) a hinge domain, (c) a transmembrane domain, and (d) a cytoplasmic domain comprising (i) optionally one or more costimulatory domains, and (ii) a signaling domain.

In certain aspects, the present disclosure provides a polynucleotide encoding a first chimeric antigen receptor (CAR) comprising (a) an extracellular target-binding domain comprising a Fibronectin Extradomain B (FN-EDB)-binding moiety, (b) a hinge domain, (c) a transmembrane domain, (d) a cytoplasmic domain comprising (i) optionally one or more costimulatory domains, (ii) a signaling domain.

In certain aspects, the present disclosure provides a polynucleotide encoding a first chimeric antigen receptor (CAR) comprising (a) an extracellular target-binding domain comprising a Fibronectin Extradomain B (FN-EDB)-binding moiety, (b) a hinge domain, (c) a transmembrane domain, (d) a cytoplasmic domain comprising (i) optionally one or more costimulatory domains, (ii) a signaling domain, and (e) a second gene.

### Extracellular Target-Binding Domain

In certain aspects, the CARs of the present disclosure comprise an extracellular target-binding domain, wherein the extracellular target-binding domain comprises an antigen-binding moiety.

The choice of antigen-binding moiety depends upon the type and number of antigens that define the surface of a target cell. For example, the antigen-binding moiety may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding moiety that specifically binds to an antigen (e.g., on a tumor cell). Non-limiting examples of cell surface markers that may act as targets for the antigen-binding moiety in the CAR of the disclosure include those associated with tumor cells.

Examples of antigens that may be targeted by the extracellular target-binding domains include, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, HER2/neu, hypoxia inducible factor (HIF-I), Ia, IL-2, IL-6, IL-8, interleukin 13 receptor α2 (IL13Rα2), insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, or fibronectin-EDB (oncofetal fibronectin, FN-EDB, EDB).

In certain embodiments, the antigen that is targeted by the extracellular target-binding domain is FN-EDB or a variant of FN-EDB.

In certain embodiments, the antigen-binding moiety can be monomeric or multimeric (e.g., homodimeric or heterodimeric), or associated with multiple proteins in a non-covalent complex. In some embodiments, the antigen-binding moiety comprises an antigen-binding polypeptide or functional variant thereof that binds to an antigen. In some embodiments, the antigen-binding polypeptide is an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In certain embodiments, the antigen-binding moiety comprises an FN-EDB-binding polypeptide or functional variant thereof. In certain embodiments, the antigen-binding moiety is an antibody or an antibody fragment that binds to FN-EDB. In certain embodiments, the FN-EDB-binding moiety is an anti-FN-EDB single chain variable fragment (scFv). In some embodiments, the anti-FN-EDB is derived from an FN-EDB specific MAb (EDB-specific scFv). In some embodiments, the anti-FN-EDB is derived from an FN-EDB specific Mab L19 (L19 scFv). The L19 antibody is a specific FN-EDB antibody described in U.S. Pat. No. 8,455,625, which is herein incorporated by reference in its entirety for all purposes. In some embodiments, the anti-FN-EDB is derived from EBD-specific antibodies such as, but not limited to, bc-1 and NJB2 (see www.abcam.com/fibronectin-antibody-bc-1-ab154210; which incorporated herein by reference in their entirety for all purposes).

In some embodiments, L19 scFV comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25. In certain embodiments, the nucleotide sequence that encodes the L19 scFV comprises the nucleotide sequence that encodes the amino acid sequence of SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25. In certain embodiments, the nucleotide sequence that encodes the L19 scFV comprises the nucleotide sequence set forth in SEQ ID: 26, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26 or 129. In certain embodiments, the L19 scFV comprises the amino acid sequence set forth in SEQ ID NO: 25. In certain embodiments, the nucleotide sequence that encodes the L19 scFV comprises the nucleotide sequence set forth in SEQ ID: 26 or 129.

In certain embodiments, the extracellular target-binding domain of the CAR is a ligand for a target cell antigen or receptor. In certain embodiments, the ligand is a natural ligand of the antigen or receptor.

### Linker Region and Hinge Domain

In certain embodiments, the CAR further comprises a linker region between the extracellular antigen-binding domain and the transmembrane domain, wherein the antigen-binding moiety, linker, and the transmembrane domain are in frame with each other.

The term “linker region” as used herein generally means any oligo- or polypeptide that functions to link the antigen-binding moiety to the transmembrane domain. A linker region can be used to provide more flexibility and accessibility for the antigen-binding moiety. A linker region may comprise up to 300 amino acids, preferably 10 to 100 amino acids and most preferably 25 to 50 amino acids. A linker region may be derived from all or part of naturally occurring molecules, such as from all or part of the extracellular region of CD8, CD4 or CD28, or from all or part of an antibody constant region. Alternatively, the linker region may be a synthetic sequence that corresponds to a naturally occurring linker region sequence, or may be an entirely synthetic linker region sequence. Non-limiting examples of linker regions which may be used in accordance to the disclosure include a part of human CD8α chain, partial extracellular domain of CD28, FcγRllla receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge domain. The hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the linker domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the linker domain comprises the CH1, CH2, CH3 and/or hinge region of the immunoglobulin. In certain embodiments, the linker domain comprises the core hinge region of the immunoglobulin. The term “core hinge” can be used interchangeably with the term “short hinge” (a.k.a “SH”). Non-limiting examples of suitable linker domains are the core immunoglobulin hinge regions listed in Table 1 (see also Wypych et al., JBC 2008 283 (23): 16194-16205, which is incorporated herein by reference in its entirety for all purposes). In certain embodiments, the linker domain is a fragment of the immunoglobulin hinge.

TABLE 1
Amino Acid Sequence of Short Hinge
Regions of IgG Immunoglobulins

| IgG | | |
|---|---|---|
| Subtype | Short Hinge Sequence | SEQ ID NO |
| IgG1 | EPKSCDKTHTCPPCP | SEQ ID NO: 27 |
| IgG1 | DLEPKSCDKTHTCPPCPDPK | SEQ ID NO: 91 |
| IgG2 | ERKCCVECPPCP | SEQ ID NO: 29 |
| IgG3 | ELKTPLGDTTHTCPRCP | SEQ ID NO: 30 |
| | (EPKSCDTPPPCPRCP)₃ | |
| IgG4 | ESKYGPPCPSCP | SEQ ID NO: 31 |

In certain embodiments, the hinge domain comprises an IgG1 hinge, or a variant thereof. In certain embodiments, the hinge domain comprises the short hinge structure of IgG1, IgG2, IgG3, or IgG4 or a variant thereof. In certain embodiments, hinge domain comprises a short hinge region and comprises the amino acid sequence set forth in SEQ ID NO: 27, 29, 30, 31, or 91, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27, 29, 30, 31, or 91. In certain embodiments, the nucleotide sequence encoding the hinge comprising the short hinge region comprises the nucleotide sequence that encodes the amino acid sequence of SEQ ID NO: 27, 29, 30, 31, or 91, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27, 29, 30, 31, or 91. In certain embodiments, the nucleotide sequence encoding the hinge comprising the short hinge region comprises the nucleotide sequence of SEQ ID NO: 28 or 92, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 28 or 92. In certain embodiments, hinge domain comprises a short hinge region and comprises the amino acid sequence set forth in SEQ ID NO: 27, 29, 30, 31, or 91. In certain embodiments, the nucleotide sequence encoding the hinge comprising the short hinge region comprises the nucleotide sequence of SEQ ID NO: 28 or 92. In certain embodiments, hinge domain is a short hinge region comprising the amino acid sequence set forth in SEQ ID NO: 27 or 91 or a variant thereof. In certain embodiments, the nucleotide sequence encoding the short hinge region comprises the nucleotide sequence of SEQ ID: 28 or 92 or a variant thereof.

In some embodiments, the hinge domain is derived from CD8a stalk or complete or partial sequences of the CD8a stalk, which are also called CD8a hinge. In some embodiments, the hinge domain derived from CD8a stalk comprises the amino acid sequence set forth in SEQ ID NO: 32, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 32. In certain embodiments, the nucleotide sequence that encodes the CD8a stalk hinge domain comprises the nucleotide sequence that encodes the amino acid sequence of SEQ ID NO: 32, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 32. In certain embodiments, the nucleotide sequence that encodes the CD8a stalk hinge domain comprises the nucleotide sequence set forth in SEQ ID NO: 33, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the CD8a stalk hinge domain comprises the amino acid sequence set forth in SEQ ID NO: 32. In certain embodiments, the nucleotide sequence that encodes the CD8a stalk hinge domain comprises the nucleotide sequence set forth in SEQ ID NO: 33.

In some embodiments, the hinge domain is derived from CD28. In some embodiments, the hinge domain derived from CD28 hinge domain comprises the amino acid sequence set forth in SEQ ID NO: 34, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 34. In certain embodiments, the nucleotide sequence that encodes the CD28 hinge domain comprises the nucleotide sequence that encodes the amino acid sequence of SEQ ID NO: 34, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 34. In certain embodiments, the nucleotide sequence that encodes the CD28 hinge domain comprises the nucleotide sequence set forth in SEQ ID NO: 35, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 35. In certain embodiments, the CD28 hinge domain comprises the amino acid sequence set forth in SEQ ID NO: 34. In certain embodiments, the nucleotide sequence that encodes the CD28 hinge domain comprises the nucleotide sequence set forth in SEQ ID NO: 35.

In some embodiments, in addition to the hinge domain, the linker region comprises additional linker amino acids to allow for extra flexibility and/or accessibility.

### Transmembrane Domain

In certain aspects, the CARs of the present disclosure comprise a transmembrane domain, fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor, CD28, CD3 epsilon, CD45, CD4, CD5, CD8, CD8α, CD9, CD16, CD22, CD33, CD37, CD40, CD64, CD80, CD86, CD134, CD137, CD154.

Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In certain embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3− γ, ζ, or η, in order to retain physical association with other members of the receptor complex.

In certain embodiments, the transmembrane domain in the CAR of the disclosure is derived from the CD28 transmembrane domain. In certain embodiments, the CD28 transmembrane domain comprises the amino acid sequence set forth in SEQ ID NO: 36, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO:36. In certain embodiments, the nucleotide sequence that encodes the CD28 transmembrane domain comprises the nucleotide sequence that encodes the amino acid sequence of SEQ ID NO: 36, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO:36. In certain embodiments, the nucleotide sequence that encodes the CD28 transmembrane domain comprises the nucleotide sequence set forth in SEQ ID NO: 37, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 37. In certain embodiments, the CD28 transmembrane domain comprises the amino acid sequence set forth in SEQ ID NO: 36. In certain embodiments, the nucleotide sequence that encodes the CD28 transmembrane domain comprises the nucleotide sequence set forth in SEQ ID NO: 37.

In certain embodiments, the transmembrane domain in the CAR of the disclosure is derived from the CD8α transmembrane domain. In certain embodiments, the CD8a transmembrane domain comprises the amino acid sequence set forth in SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD8a transmembrane domain comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD8a transmembrane domain comprises the nucleotide sequence set forth in SEQ ID NO: 39, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 39. In certain embodiments, the CD8a transmembrane domain comprises the amino acid sequence set forth in SEQ ID NO: 38. In certain embodiments, the nucleotide sequence that encodes the CD8a transmembrane domain comprises the nucleotide sequence set forth in SEQ ID NO: 39.

### Cytoplasmic Domain

In certain aspects, CARs of the present disclosure comprise a cytoplasmic domain, which comprises one or more costimulatory domains and one or more signaling domains. The cytoplasmic domain, which comprises one or more costimulatory domains and one or more signaling domains, is responsible for activation of at least one of the normal effector functions of the lymphocyte in which the CAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcRβ, CD3δ, CD3ε, CD3γ, CD3ζ, CD5, CD22, CD226, CD66d, CD79A, and CD79B. In some embodiments, the CAR of the present disclosure comprises a signaling domain derived from CD3ζ.

In certain embodiments, the lymphocyte activation domain in the CAR of the disclosure is designed to comprise the signaling domain of CD35. In certain embodiments, the CD35 signaling domain comprises the amino acid sequence set forth in SEQ ID NO: 57 or 59 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 57 or 59. In certain embodiments, the nucleotide sequence that encodes the CD35 signaling domain comprises the nucleotide sequence that encodes the amino acid sequence of SEQ ID NO: 57 or 59, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 57 or 59. In certain embodiments, the nucleotide sequence that encodes the CD3ζ signaling domain comprises the nucleotide sequence set forth in SEQ ID NO: 58, 60, or 123-126, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 58, 60, or 123-126. In certain embodiments, the CD3ζ signaling domain comprises the amino acid sequence set forth in SEQ ID NO: 57 or 59. In certain embodiments, the nucleotide sequence that encodes the CD3ζ signaling domain comprises the nucleotide sequence set forth in SEQ ID NO: 58, 60, or 123-126.

Non-limiting examples of costimulatory domains which can be used in the CARs of the present disclosure include, those derived from 4-1BB (CD137), CD28, CD40, ICOS, CD134 (OX-40), BTLA, CD27, CD30, GITR, CD226, CD79A, and HVEM. In some embodiments, the CAR of the present disclosure comprises one costimulatory domain. In some embodiments, the CAR of the present disclosure comprises a costimulatory domain derived from 4-1BB. In some embodiments, the CAR of the present disclosure comprises a costimulatory domain derived from CD28.

In some embodiments, the CAR of the present disclosure comprises two or more costimulatory domains. In certain embodiments, the CAR of the present disclosure comprises two, three, four, five, six or more costimulatory domains. In some embodiments, the CAR of the present disclosure comprises a costimulatory domain derived from 4-1BB and a costimulatory domain derived from CD28.

In certain aspects, the CARs of the present disclosure comprise a cytoplasmic domain, which comprises a lymphocyte activation domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the lymphocyte activation domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and costimulatory domain(s) can be in any order. In some embodiments, the signaling domain is upstream of the costimulatory domains. In some embodiments, the signaling domain is downstream from the costimulatory domains. In the cases where two or more costimulatory domains are included, the order of the costimulatory domains could be switched.

In some embodiments, the costimulatory domain derived from CD28 comprises the amino acid sequence set forth in SEQ ID NO: 40, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 40. In certain embodiments, the nucleotide sequence that encodes the CD28 costimulatory domain comprises the nucleotide sequence that encodes the amino acid sequence of SEQ ID NO: 40, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 40. In certain embodiments, the nucleotide sequence that encodes the CD28 costimulatory domain comprises the nucleotide sequence set forth in SEQ ID NO: 41, 42, 43, or 44, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 41, 42, 43, or 44. In certain embodiments, the CD28 costimulatory domain comprises the amino acid sequence set forth in SEQ ID NO: 40. In certain embodiments, the nucleotide sequence that encodes the CD28 costimulatory domain comprises the nucleotide sequence set forth in SEQ ID NO: 41, 42, 43, or 44.

In some embodiments, the costimulatory domain derived from 4-1BB comprises the amino acid sequence set forth in SEQ ID NO: 45, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 45. In certain embodiments, the nucleotide sequence that encodes the 4-1BB costimulatory domain comprises the nucleotide sequence that encodes the amino acid sequence of SEQ ID NO: 45, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 45. In certain embodiments, the nucleotide sequence that encodes the 4-1BB costimulatory domain comprises the nucleotide sequence set forth in SEQ ID NO: 46, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 46. In certain embodiments, the 4-1BB costimulatory domain comprises the amino acid sequence set forth in SEQ ID NO: 45. In certain embodiments, the nucleotide sequence that encodes the 4-1BB costimulatory domain comprises the nucleotide sequence set forth in SEQ ID NO: 46.

In some embodiments, the costimulatory domain derived from OX40 comprises the amino acid sequence set forth in SEQ ID NO: 47, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 47. In certain embodiments, the nucleotide sequence that encodes the OX40 costimulatory domain comprises the nucleotide sequence that encodes the amino acid sequence of SEQ ID NO: 47, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 47. In certain embodiments, the nucleotide sequence that encodes the OX40 costimulatory domain comprises the nucleotide sequence set forth in SEQ ID NO: 48 or 127, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48 or 127. In certain embodiments, the OX40 costimulatory domain comprises the amino acid sequence set forth in SEQ ID NO: 47. In certain embodiments, the nucleotide sequence that encodes the OX40 costimulatory domain comprises the nucleotide sequence set forth in SEQ ID NO: 48 or 127.

In certain embodiments, the MyD88 polypeptide or functional fragment thereof in the CAR of the disclosure is designed to comprise the co-stimulatory domain of MyD88, or variant thereof. In certain embodiments, the MyD88 functional fragment comprises the amino acid sequence set forth in SEQ ID NO: 49, 51, or 53, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49, 51, or 53. In certain embodiments, the nucleotide sequence encoding the MyD88 functional fragment comprises the nucleotide sequence that encodes the amino acid sequence of SEQ ID NO: 49, 51, or 53, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49, 51, or 53. In certain embodiments, the nucleotide sequence encoding the MyD88 functional fragment comprises the nucleotide sequence set forth in SEQ ID NO: 50, 52, or 54, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 50, 52, or 54. In certain embodiments, the MyD88 functional fragment comprises the amino acid sequence set forth in SEQ ID NO: 49, 51, or 53. In certain embodiments, the nucleotide sequence that encodes the MyD88 functional fragment comprises the nucleotide sequence set forth in SEQ ID NO: 50, 52, or 54.

In certain embodiments, the CD40 polypeptide or functional fragment thereof in the CAR of the disclosure is designed to comprise the CD40 cytoplasmic polypeptide region. In certain embodiments, the CD40 cytoplasmic polypeptide region comprises the amino acid sequence set forth in SEQ ID NO: 55 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 55. In certain embodiments, the nucleotide sequence encoding the CD40 cytoplasmic polypeptide region comprises the nucleotide sequence that encodes the amino acid sequence of SEQ ID NO: 55, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 55. In certain embodiments, the nucleotide sequence encoding the CD40 cytoplasmic polypeptide region comprises the nucleotide sequence set forth in SEQ ID NO: 56, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 56. In certain embodiments, the CD40 cytoplasmic polypeptide region comprises the amino acid sequence of SEQ ID NO: 55. In certain embodiments, the nucleotide sequence encoding the CD40 cytoplasmic polypeptide region comprises the nucleotide sequence set forth in SEQ ID NO: 56. Leader Sequence

In certain aspects, the CAR of the present disclosure comprises a leader sequence. The leader sequence may be positioned amino-terminal to the extracellular target-binding domain. The leader sequence may be optionally cleaved from the antigen-binding moiety during cellular processing and localization of the CAR to the cellular membrane.

In some embodiments, the leader sequence may be derived from human immunoglobulin heavy chain variable region. In some embodiments, the leader sequence comprises the amino acid sequence set forth in SEQ ID NO: 61 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 61. In certain embodiments, the nucleotide sequence encoding the leader sequence comprises the nucleotide sequence that encodes the amino acid sequence of SEQ ID NO: 61, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 61. In certain embodiments, the nucleotide sequence encoding the leader sequence comprises the sequence set forth in SEQ ID: 62, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 62 or 128. In certain embodiments, the leader sequence comprises the amino acid sequence of SEQ ID NO: 61. In certain embodiments, the nucleotide sequence encoding the leader sequence comprises the nucleotide sequence set forth in SEQ ID NO: 62 or 128.

In some embodiments, the leader sequence may be derived from CD8a. In some embodiments, the leader sequence comprises the amino acid sequence set forth in SEQ ID NO: 63 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 63. In certain embodiments, the nucleotide sequence encoding the leader sequence comprises the nucleotide sequence that encodes the amino acid sequence of SEQ ID NO: 63, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 63. In certain embodiments, the nucleotide sequence encoding the leader sequence comprises the sequence set forth in SEQ ID NO: 64, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 64. In certain embodiments, the leader sequence comprises the amino acid sequence of SEQ ID NO: 63. In certain embodiments, the nucleotide sequence encoding the leader sequence comprises the nucleotide sequence set forth in SEQ ID NO: 64.

### Additional Genes

In addition to the CAR construct, the CAR may further comprise at least one additional gene that encodes an additional peptide. Examples of additional genes can include a transduced host cell selection marker, an in vivo tracking marker, a cytokine, a suicide gene, or some other functional gene. In certain embodiments, the functional additional gene can induce the expression of another molecule. In certain embodiments, the functional additional gene can increase the safety of the CAR. For example, the CAR construct may comprise an additional gene which is truncated CD19 (tCD19). The tCD19 can be used as a tag. Expression of tCD19 may also help determine transduction efficiency. As another example, the CAR construct may comprise an additional gene which is a synNotch receptor. Once activated, the synNotch receptor can induce the expression of a target gene (e.g., a second CAR and/or bispecific molecule).

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules).

In certain embodiments, the CAR comprises at least one additional gene (i.e., a second gene). In certain embodiments, the CAR comprises one second gene. In other embodiments, the CAR comprises two additional genes (i.e., a third gene). In yet another embodiment, the CAR comprises three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc)

Non-limiting examples of classes of additional genes that can be used to increase the effector function of CAR containing host cells, include (a) secretable cytokines (e.g., but not limited to, IL-7, IL-12, IL-15, IL-18), (b) membrane bound cytokines (e.g., but not limited to, IL-15), (c) chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), or (i) a second CAR.

In some embodiments, the additional gene sequence may be derived from tCD19. In some embodiments, the tCD19 sequence comprises the amino acid sequence set forth in SEQ ID NO: 65 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 65. In certain embodiments, the nucleotide sequence encoding the tCD19 sequence comprises the nucleotide sequence that encodes the amino acid sequence of SEQ ID NO: 65, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 65. In certain embodiments, the nucleotide sequence encoding the tCD19 sequence comprises the sequence set forth in SEQ ID NO: 66, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 66. In certain embodiments, the tCD19 sequence comprises the amino acid sequence of SEQ ID NO: 65. In certain embodiments, the nucleotide sequence encoding the tCD19 sequence comprises the nucleotide sequence set forth in SEQ ID NO: 66.

In some embodiments, the additional gene sequence may be a synNotch receptor. Example 4 provides a non-limiting example of a synNotch receptor, wherein the receptor comprises the transcription factor Gal4-VP64 as an intracellular domain. In other non-limiting embodiments, other intracellular domains include Gal4-KRAB, FFHD1-VP64, or tTA.

In some embodiments, the synNotch receptor sequence comprises the amino acid sequence set forth in SEQ ID NO: 67, 93, or 121, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 67, 93, or 121. In certain embodiments, the nucleotide sequence encoding the synNotch receptor sequence comprises the nucleotide sequence that encodes the amino acid sequence of SEQ ID NO: 67, 93, or 121, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 67, 93, or 121. In certain embodiments, the nucleotide sequence encoding the synNotch receptor sequence comprises the sequence set forth in SEQ ID NO: 68, 94, or 122, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 68, 95, or 122. In certain embodiments, the synNotch receptor sequence comprises the amino acid sequence of SEQ ID NO: 67, 93, or 121. In certain embodiments, the nucleotide sequence encoding the synNotch receptor sequence comprises the nucleotide sequence set forth in SEQ ID NO: 68, 95, or 122. An antigen-binding moiety (e.g., anti-EDB scFv (SEQ ID NO: 25)) is attached to the synNotch receptor, which will bind to the target antigen.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105 (11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105 (11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

Accordingly, in some embodiments, the polynucleotide encoding a CAR of the present disclosure further comprises a sequence encoding a CD20 polypeptide. In some embodiments, the CD20 polypeptide comprises the amino acid sequence set forth in SEQ ID NO: 84, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 84. In certain embodiments, the nucleotide sequence that encodes the CD20 polypeptide comprises the nucleotide sequence that encodes the amino acid sequence of SEQ ID NO: 84, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 84. In certain embodiments, the nucleotide sequence that encodes the CD20 polypeptide comprises the nucleotide sequence set forth in SEQ ID NO: 85 or 86, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 85 or 86. In certain embodiments, the CD20 polypeptide comprises the amino acid sequence set forth in SEQ ID NO: 84. In certain embodiments, the nucleotide sequence that encodes the CD20 polypeptide comprises the nucleotide sequence set forth in SEQ ID NO.: 85 or 86.

In some embodiments, the sequence encoding an additional gene is operably linked to the sequence encoding CAR via a sequence encoding a self-cleaving peptide and/or an Internal Ribosome Entry Site (IRES) as disclosed herein.

Non-limiting examples of self-cleaving peptide sequences includes Thoseaasigna virus 2A (T2A; AEGRGSLLTCGDVEENPGP, SEQ ID NO: 69, EGRGSLLTCGDVEENPGP, SEQ ID NO: 71, or GSGEGRGSLLTCGDVEENPGP, SEQ ID NO: 73); the foot and mouth disease virus (FMDV) 2A sequence (F2A; GSGSRVTELLYRMKRAETYCPRPLLAIHPTEARHKQKIVAPVKQLLNFDLLKLAGDV ESNPGP, SEQ ID NO: 74), Sponge (Amphimedon queenslandica) 2A sequence (LLCFLLLLLSGDVELNPGP, SEQ ID NO: 75; or HHFMFLLLLLAGDIELNPGP, SEQ ID NO: 76); acorn worm 2A (Saccoglossus sequence kowalevskii) (WFLVLLSFILSGDIEVNPGP, SEQ ID NO: 77); amphioxus (Branchiostoma floridae) 2A sequence (KNCAMYMLLLSGDVETNPGP, SEQ ID NO: 78; or MVISQLMLKLAGDVEENPGP, SEQ ID NO: 79); porcine teschovirus-1 2A sequence (P2A; GSGATNFSLLKQAGDVEENPGP, SEQ ID NO: 80); and equine rhinitis A virus 2A sequence (E2A; GSGQCTNYALLKLAGDVESNPGP, SEQ ID NO: 81). In some embodiments, the separation sequence is a naturally occurring or synthetic sequence. In certain embodiments, the separation sequence includes the 2A consensus sequence D-X-E-X-NPGP (SEQ ID NO: 82), in which X is any amino acid residue.

Alternatively, an Internal Ribosome Entry Site (IRES) may be used to link the CAR and the additional gene. IRES is an RNA element that allows for translation initiation in a cap-independent manner. IRES can link two coding sequences in one bicistronic vector and allow the translation of both proteins in cells.

In some embodiments, the self-cleaving 2A peptide is a T2A peptide and comprises the amino acid sequence set forth in SEQ ID NO: 69 or 71. In some embodiments, the sequence encoding the T2A peptide comprises the nucleotide sequence SEQ ID NO: 70, 72 or 83.

In certain embodiments, the host cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8a, CD28, portions of a T-cell receptor, or an antigen-binding moiety (e.g., scFv) linked to a MyD88, CD40, and/or other signaling molecules.

In certain embodiments, the fusion protein comprises an extracellular target-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., Cd3ζ). In certain embodiments, the at least one co-stimulatory protein can be a MyD88 polypeptide or functional fragment thereof (as described above), and/or a CD40 cytoplasmic polypeptide region or a functional fragment thereof (as described above).

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., Cd3ζ). In certain embodiments, the at least one portion of the fusion protein can be a MyD88 polypeptide or functional fragment thereof (as described above), and/or a CD40 cytoplasmic polypeptide region or a functional fragment thereof (as described above).

Non-limiting examples of fusion proteins include, but are not limited to, the constructs in the publication of PCT/US19/32786 and WO2016073875, which are incorporated herein by reference in its entirety for all purposes.

In certain embodiments, the fusion proteins are introduced into the host cell on a separate vector from the CAR. In certain embodiments, the fusion proteins are introduced into the host cell on the same vector as the CAR. In certain embodiments, the fusion proteins are introduced into the host cell on the same vector as the CAR but separated by a separation sequence such as 2A.

### Non-Limited Examples of CARS

In certain embodiments, the CAR can be encoded by one polypeptide chain. In certain embodiments, the CAR of the disclosure is encoded by a nucleotide sequence comprising the nucleotides sequence of SEQ ID NO: 2, 4, 6, 8, 10, 12, 14, 16, 18, 20, 22, 24, 96, 98, 100, 102, 104, 106, 108, 110, 112, 114, 116, 118, or 120, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2, 4, 6, 8, 10, 12, 14, 16, 18, 20, 22, 24, 96, 98, 100, 102, 104, 106, 108, 110, 112, 114, 116, 118, or 120. In certain embodiments, the CAR is encoded by the nucleotide sequence that encodes the amino acid sequence of SEQ ID NO: 1, 3, 5, 7, 9, 11, 13, 15, 17, 19, 21, 23, 95, 97, 99, 101, 103, 105, 107, 109, 111, 113, 115, 117, or 119. In another embodiment, the CAR comprises the amino acid sequence of SEQ ID NO: 1, 3, 5, 7, 9, 11, 13, 15, 17, 19, 21, 23, 95, 97, 99, 101, 103, 105, 107, 109, 111, 113, 115, 117, or 119, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1, 3, 5, 7, 9, 11, 13, 15, 17, 19, 21, 23, 95, 97, 99, 101, 103, 105, 107, 109, 111, 113, 115, 117, or 119. In another embodiment, the CAR comprises the amino acid sequence of SEQ ID NO: 1, 3, 5, 7, 9, 11, 13, 15, 17, 19, 21, 23, 95, 97, 99, 101, 103, 105, 107, 109, 111, 113, 115, 117, or 119.

In certain embodiments, the CAR can be encoded by two polypeptide chains. For example, the first polypeptide chain can encode an extracellular target-binding domain comprising an FN-EDB-binding moiety, a transmembrane domain, and a short cytoplasmic tail, and the second polypeptide chain can encode a short extracellular domain, a transmembrane domain, and a cytoplasmic domain comprising a lymphocyte activation domain, a MyD88 polypeptide or functional fragment thereof, and a CD40 cytoplasmic polypeptide region or a functional fragment thereof. Each domain is described in greater detail above. In certain embodiments, both polypeptides can interact via their respective transmembrane domain.

In various embodiments, the polynucleotide encoding a CAR is a DNA molecule. In various embodiments, the polynucleotide encoding a CAR is an RNA molecule.

In one aspect, the present disclosure provides CAR polypeptides encoded by a polynucleotide described above.

### Vectors

The present disclosure provides recombinant vectors comprising a polynucleotide encoding a CAR comprising polynucleotides encoding the proteins disclosed above. In certain embodiments, the polynucleotide is operatively linked to at least one regulatory element for expression of the chimeric antigen receptor.

In certain embodiments, recombinant vectors of the disclosure comprise the nucleotide sequence of SEQ ID NO: 2, 4, 6, 8, 10, 12, 14, 16, 18, 20, 22, 24, 96, 98, 100, 102, 104, 106, 108, 110, 112, 114, 116, 118, or 120, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2, 4, 6, 8, 10, 12, 14, 16, 18, 20, 22, 24, 96, 98, 100, 102, 104, 106, 108, 110, 112, 114, 116, 118, or 120. In certain embodiments, recombinant vectors comprise a nucleotide sequence that encodes the amino acid sequence of SEQ ID NO: 1, 3, 5, 7, 9, 11, 13, 15, 17, 19, 21, 23, 95, 97, 99, 101, 103, 105, 107, 109, 111, 113, 115, 117, or 119, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1, 3, 5, 7, 9, 11, 13, 15, 17, 19, 21, 23, 95, 97, 99, 101, 103, 105, 107, 109, 111, 113, 115, 117, or 119.

In certain embodiments, the recombinant vector comprises a polynucleotide encoding a CAR, wherein the polynucleotide is operatively linked to at least one additional gene. In some embodiments, the additional gene is a synNotch receptor. In some embodiments, the additional gene is a tCD19.

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. The viral vector may be a plasmid or a transposon (such as a PiggyBac- or a Sleeping Beauty transposon).

In certain embodiments, the polynucleotide encoding the CAR is operably linked to at least a regulatory element. The regulatory element can be capable of mediating expression of the CAR in the host cell. Regulatory elements include, but are not limited to, promoters, enhancers, initiation sites, polyadenylation (polyA) tails, IRES elements, response elements, and termination signals. In certain embodiments, the regulatory element regulates CAR expression. In certain embodiments, the regulatory element increased the expression of the CAR. In certain embodiments, the regulatory element increased the expression of the CAR once the host cell is activated. In certain embodiments, the regulatory element decreases expression of the CAR. In certain embodiments, the regulatory element decreases expression of the CAR once the host cell is activated.

### CAR-Modified Host Cells

In one aspect, the present disclosure provides an isolated host cell comprising a polynucleotide or a recombinant vector described herein. In one aspect, the present disclosure provides an isolated host cell comprising a CAR described herein. In various embodiments, the isolated host cell further comprises a second antigen targeting molecule (e.g., a second CAR or a bispecific molecule) that targets a TAA expressed on the target tumor cell. In certain embodiments, the isolated host cell further comprises a second CAR that targets a TAA expressed on the target tumor cell. In certain embodiments, the isolated host cell further comprises a bispecific molecule that targets a TAA expressed on the target tumor cell. In certain embodiments, the bispecific molecule is a bispecific T-cell engager (BiTE) or a bispecific antibody.

In certain embodiments, the TAA is 5T4, αᵥβ₆ integrin, B7-H3, B7-H6, CAIX, CD19, CD20, CD22, CD30, CD33, CD44, CD44v6, CD44v7/8, CD70, CD123, CD138, CD171, CEA, Claudin-6 or -18, CSPG4, EGFR, EGFR family including ErbB2 (HER2), EGFRVIII, EGP2, EGP40, EPCAM, EphA2, EpCAM, FAP, fetal AchR, FRα, GD2, GD3, MAGE1, NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, Survivin, TAG72, TEMs, or VEGFR2.

In various embodiments, the host cell is an immune cell. The immune cell may be a T-cell or a natural killer (NK) cell.

In various embodiments, the host cell is a T-cell. T-cells may include, but are not limited to, thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+CD8+ T-cell, or any other subset of T-cells. Other illustrative populations of T-cells suitable for use in particular embodiments include naive T-cells memory T-cells, and NKT cells.

In some embodiments, the T-cell is selected from a CD8+ T-cell, a CD4+ T-cell, a cytotoxic T-cell, an αβ T-cell receptor (TCR) T-cell, a natural killer T (NKT) cell, a γδ T-cell, a memory T-cell, a T-helper cell, and a regulatory T-cell (Treg).

In various embodiments, the host cell is a NK cell. NK cell refers to a differentiated lymphocyte with a CD3− CD16+, CD3− CD56+, CD16+CD56+ and/or CD57+ TCR-phenotype.

In various embodiments, the host cell has been activated and/or expanded ex vivo.

In various embodiments, the host cell is an allogeneic cell. In various embodiments, the host cell is an autologous cell.

In some embodiments, the host cell is isolated from a subject having a tumor. In some embodiments, the tumor can be found within, but not limited to, breast tissue, prostate tissue, bladder tissue, oral and/or dental tissue, head and/or neck tissue, colorectal tissue, lung tissue, brain tissue, skin, lymph nodes, and bone. In some embodiments, the tumor is a cancer. In some embodiments, the cancer can be, but not limited to, breast cancer, prostate cancer, bladder cancer, oral squamous cell carcinoma, head and/or neck squamous cell carcinoma, colorectal cancer, lung cancer, brain tumors, melanoma, bone, pediatric solid tumors and brain tumors, and/or lymphoma.

In certain embodiments, the host cell is isolated from a subject having a tumor, wherein one or more cells of the tumor cells express FN-EDB. Non-limiting examples of tumor cells that express FN-EDB include breast tumor cells, prostate tumor cells, bladder tumor cells, oral squamous cell carcinoma, head and neck squamous cell carcinoma, colorectal tumor cells, lung tumor cells, brain tumors, melanoma, bone, pediatric solid tumors and brain tumors, lymphoma, plasmacytoma, mantel cell lymphoma, lymphocytic lymphoma, marginal zone lymphoma, lymphoplasmocytic lymphoma, follicular lymphoma, diffuse large B-cell lymphoma (DLBCL), B-lymphoblastic lymphoma, anaplastic large T-cell lymphoma, T lymphoblastic lymphoma, peripheral T-NHL (NOS), classical Hodgkin lymphoma (cHL), chronic myelogenous leukemia, chronic idiopathic myelofibrosis, essential thrombocythemia, polycythemia vera, and/or acute myeloblastic leukemia.

In some embodiments, the host cell is derived from a blood, marrow, tissue, or a tumor sample.

In one aspect, the present disclosure provides a method of generating an isolated host cell described herein. The method includes genetically modifying the host cell with a polynucleotide encoding a CAR and optionally an additional gene (e.g., synNotch, tCD19), and/or the recombinant vector comprising the polynucleotide encoding a CAR and optionally a safety switch (e.g., CD20 polypeptide). The genetically modifying step may be conducted in vivo or ex vivo. In some embodiments, the genetically modifying step is conducted ex vivo. The method may further include activation and/or expansion of the host cell ex vivo before, after and/or during the genetic modification.

### Isolation/Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T-cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T-cell based host cells can be activated by binding to an agent that activates CD35.

In other embodiments, a CD2-binding agent may be used to provide a primary stimulation signal to the T-cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab) 2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab) 2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD35 agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a CAR described above. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the CAR, the CAR polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. In some embodiments, the non-viral vector is a transposon. Exemplary transposons hat can be used in the present disclosure include, but are not limited to, a sleeping beauty transposon and a PiggyBac transposon.

Nucleic acid vaccines can be used to transfer CAR polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244 (4910): 1342-1344, 1989, both of which are incorporated herein by reference in their entity. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this disclosure include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5 (5): 1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7 (8): 2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87 (9): 3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present disclosure include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+guide RNA) capable of inducing a double strand break in a target DNA sequence is introduced to a host cell, along with a donor polynucleotide encoding a CAR of the present disclosure and optionally an additional protein (e.g., synNotch or tCD19).

### Expansion/Proliferation

After the host cells are activated and transduced, the cells are cultured to proliferate. T-cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T-cells can include interleukins, such as IL-2, IL-7, IL-15, or IL-21 (see for example Cornish et al. 2006, Blood. 108 (2): 600-8, Bazdar and Sieg, 2007, Journal of Virology, 2007, 81 (22): 12670-12674, Battalia et al, 2013, Immunology, 139 (1): 109-120). Other illustrative examples for agents that may be used for the expansion of T-cells are agents that bind to CD8, CD45 or CD90, such as αCD8, αCD45 or αCD90 antibodies. Illustrative examples of T-cell population including antigen-specific T-cells, T helper cells, cytotoxic T-cells, memory T-cell (an illustrative example of memory T-cells are CD62L|CD8| specific central memory T-cells) or regulatory T-cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78 (12): 3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, B2M, βAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription n activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In some embodiments, the compositions comprise one or more polypeptides of the CARs and other related molecules (e.g., synNotch receptor, second CAR or bispecific molecule), polynucleotides, vectors comprising same, and cell compositions, as disclosed herein. Compositions of the present disclosure include, but are not limited to pharmaceutical compositions.

In one aspect, the present disclosure provides a pharmaceutical composition comprising a polynucleotide or a recombinant vector described herein, and a pharmaceutically accepted carrier and/or excipient.

In another aspect, the present disclosure provides pharmaceutical composition comprising the CAR-modified host cells described herein and a pharmaceutically acceptable carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising CAR-modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising CAR-modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the CAR-modified host cells may be mixed with substances that adhere or penetrate then prior to their administration, e.g., but not limited to, nanoparticles.

### Therapeutic Methods

In one aspect, the present disclosure provides a method for treating a tumor in a subject in need thereof. A therapeutically effective amount of the CAR-modified host cells described herein or the pharmaceutical composition comprising the host cells is administered to the subject.

The term “tumor” refers to a benign or malignant abnormal growth of tissue. The term “tumor” includes cancer. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and β-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-O-911910-19-3); The Merck Manual of Diagnosis and Therapy, 20th Edition, § on Hematology and Oncology, published by Merck Sharp & Dohme Corp., 2018 (ISBN 978-O-911-91042-1) (2018 digital online edition at internet website of Merck Manuals); and SEER Program Coding and Staging Manual 2016, each of which are incorporated by reference in their entirety for all purposes.

In some embodiments, host cells modified with a FN-EDB-binding CAR, or pharmaceutical compositions thereof, are administered to a subject to treat a tumor expressing FN-EDB.

In some embodiments, host cells modified with an TAA-binding CAR, or pharmaceutical compositions thereof, are administered to a subject to treat a tumor expressing the TAA.

In some embodiments, host cells modified with an TAA-binding bispecific molecule (e.g., a BiTE or bispecific antibody), or pharmaceutical compositions thereof, are administered to a subject to treat a tumor expressing the TAA.

In cases where the CAR-modified host cells also express a CD20 polypeptide, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments, the therapeutic method of the present disclosure includes one or more of the following steps: (a) isolating immune cells from the subject or donor; (b) modifying the immune cells ex vivo with a polynucleotide encoding a CAR and optionally an additional protein, a second CAR and/or a bispecific molecule, or a recombinant vector comprising the same; (c) optionally, expanding and/or activating the modified immune cells before, after and/or during step (b); (d) introducing a therapeutically effective amount of the modified immune cells into the subject, and (e) in cases when the modified immune cells comprise the CD20 suicide switch, optionally, administering an anti-CD20 antibody to the subject, wherein the anti-CD20 antibody is administered in an amounts effective for removal of the modified immune cells from the subject. The immune cells may be T-cells and/or NK cells.

In some embodiments, the modified host cell is an autologous cell. In some embodiments, the modified host cell is an allogeneic cell. In cases where the host cell is isolated from a donor, the method may further include a method to prevent graft vs host disease (GVHD) and the host cell rejection.

In some embodiments of any of the therapeutic methods described above, the composition is administered in a therapeutically effective amount. The dosages of the composition administered in the methods of the disclosure will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10^{8.} cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the disclosure can be combined with other therapies that block inflammation (e.g., via blockage of IL1, INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the disclosure can also be combined with other treatments such as midostaurin, enasidenib, or a combination thereof.

Therapeutic methods of the disclosure can be combined with additional immunotherapies and therapies. For example, when used for treating tumors, the compositions of the disclosure can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present disclosure there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

The following materials and methods were used in Examples 1-3.

### Tumor Cell Lines

The U87 (glioma), A549 (lung cancer), and A673 (Ewing sarcoma) cell lines were purchased from the American Type Tissue Collection (ATCC). The lung metastatic osteosarcoma cell line LM7 was provided by Dr. Eugenie Kleinerman (MD Anderson Cancer Center, Houston, TX). Primary fibroblast cell lines have been previously established (62). The generation of the A549 cell line expressing an enhanced green fluorescence protein firefly luciferase fusion gene (GFP.ffluc) was previously described (61). All cell lines were grown in DMEM or RPMI (GE Healthcare Life Sciences HyClone Laboratories) supplemented with 10% fetal bovine serum (FBS; GE Healthcare Life Sciences HyClone) and 2 mM Glutamax (Invitrogen). Cell lines were authenticated using the ATCC's human STR profiling cell authentication service and routinely checked for Mycoplasma by the MycoAlert Mycoplasma Detection Kit (Lonza).

### Generation of CAR T-Cells

The generation of the SFG retroviral vectors encoding the EphA2-CAR with a CD28 costimulatory domain, or GFP.ffluc have been previously described (38). In-Fusion cloning (Takara Bio) was used to generate the EDB-CAR with a CD28 costimulatory domain. This retroviral vector contains a CAR.CD28ζ expression cassette, a 2A sequence, and truncated CD19 (38). The EDB-specific single chain variable fragment (scFv) was derived from the human MAb L19 (28) and synthesized by GeneArt (Thermo Fisher Scientific). RD114 -pseudotyped retroviral particles were generated by transient transfection of 293T cells as previously described (38). Supernatants were collected 48 hours after transfection, filtered, and snap frozen for later transduction of T-cells.

Human peripheral blood mononuclear cells (PBMCs) were obtained from whole blood of healthy donors under IRB-approved protocols at SJCRH. CAR T-cells were generated as described in (61). Briefly, Lymphoprep (Abbott Laboratories) isolated PBMCs were stimulated on treated non-tissue culture 24-well plates, which were precoated with CD3 and CD28 antibodies (BD). Recombinant human IL-7 and IL-15 (IL-7:10 ng/ml; IL-15:5 ng/ml; PeproTech) were added to cultures the next day. On day 2, CD3/CD28-stimulated T-cells (2.5×10⁵ cells/well) were transduced with RD114-pseudotyped retroviral particles on RetroNectin (Clontech)-coated plates in the presence of IL-7 and IL-15. On day 5, transduced T-cells were transferred into new tissue culture 24-well plates and subsequently expanded with IL-7 and IL-15. Non-transduced (NT) T-cells were prepared in the same way except for no retrovirus was added. For the generation of GFP.ffluc-expressing EDB-CAR T-cells, activated T-cells were transduced with two retroviral vectors. All experiments were performed 7-14 days post-transduction. Biological replicates were performed using PBMCs from different healthy donors.

### Flow Cytometry

A FACSCanto II (BD) instrument was used to acquire flow cytometry data, which was analyzed using FlowJo v10 (FlowJo). For surface staining, samples were washed with and stained in PBS (Lonza) with 1% FBS (HyClone). For all experiments, matched isotypes or known negatives (e.g. NT T-cells) served as gating controls. LIVE/DEAD® Fixable Aqua Dead Cell Stain Kit (Invitrogen) was used as a viability dye. T-cells were evaluated for CAR expression at multiple time points post-transduction with an anti-human IgG, F(ab′) 2 fragment specific-AF647 (Jackson ImmunoResearch). Transduction was also confirmed with anti-CD19-PE (clone J3-119, Beckman Coulter). T-cells phenotype was established with several different panels using combinations of antibodies that are listed in the supplemental data section.

The following antibodies were used for FACS analysis: CD3-APC (clone UTCH1, Beckman Coulter), CD4-Pacific Blue (clone SK3, BioLegend) CD8-PerCPCy5.5 (clone SK1, BioLegend), CD19-PE (clone J3-119, Beckman Coulter), CD19-BV421 (clone HIB19, BD), CCR7-AF488 (clone G043H7, BioLegend), CD45RA-APC-H7 (clone HI100, BD), LAG3-FITC (clone 11C3C65, BioLegend), TIM3-PE-Cy7 (clone F38-2E2, BioLegend), PD1-PE (clone EH12.2H7, BioLegend), and F(ab′) Goat IgG (AF647: 005600003, Jackson Labs).

### Real-Time PCR

mRNA extraction from 1×10⁶ to 1×10⁷ cells was performed using the Maxwell RSC SimplyRNA Blood kit (Promega AS1380) on a Maxwell RSC. RT-qPCR was performed according to the manufacturer's instruction using a one-step kit (ThermoFisher). Previously published sequences were used for EDB PCR primers (63), and GAPDH PCR primers were purchased from Life Technologies. Reactions were completed on the Applied Bioscience QuantStudio 6 Flex and analyzed using QuantStudio software.

### Co-Culture Assay

1×10⁶ T-cells were co-cultured with no tumor cells or 5×10⁵ LM7, U87, or A549 cells without the provision of exogenous cytokine. For recombinant protein studies, rhFN-EDB (Abcam) was incubated at decreasing concentrations for 3 hours at 37° C. Plates were washed and 5×10⁵ T-cells were plated. 24 and 48 hours later, supernatant was collected and frozen for later analysis. Cytokines were measured using IFNγ and IL-2 ELISA kits (R&D Systems).

### MTS Assay

A CellTiter96® AQueous One Solution Cell Proliferation Assay (Promega) was utilized to assess CAR T-cell cytotoxicity. In a tissue culture-treated 96-well plate, 12,500 U87, and A549, or 15,000 LM7 cells and primary fibroblasts were co-cultured with serial dilutions of T-cells. Media and tumor cells alone served as controls. Each condition was plated in triplicate. After 5 days, the media and T-cells were removed by gently pipetting up and down to avoid disrupting adherent tumor cells. CellTiter96® AQueous One Solution Reagent (phenazine ethosulfate) in RPMI-10% FBS was added to each well and incubated at 37° C., 5% CO2 for 2 hours. The absorbance at 492 nm was measured using an Infinite® 200 Pro MPlex plate reader (Tecan) to assess the number of viable cells in each well. Percent live tumor cells were determined by the following formula: (sample-media alone)/(tumor alone-media alone)*100.

### Xenograft Mouse Models

Animal experiments followed a protocol approved by SJCRH Institutional Animal Care and Use Committee. All experiments utilized 6-8-week NOD-scid IL2Rgammanull (NSG) mice obtained from SJCRH NSG colony. Subcutaneous tumor models: Mice were injected s.c. with 2×10⁶ tumor cells in Matrigel (Corning; 1:1 diluted in PBS). On day 7, mice received a single i.v. dose of 1×10⁶ T-cells via tail vein injection. Tumor growth was assessed by serial caliper measurements. Mice were euthanized when i) they met physical euthanasia criteria (significant weight loss, signs of distress), ii) the tumor burden was approximately 3,000 mm³, or iii) recommended by St. Jude veterinary staff. Intravenous tumor models: Mice were injected i.v. with 2×10⁶ tumor cells via tail vein injection, and on day 7 received a single i.v. dose of 1×10⁶ T-cells. Mice were euthanized when they reached i) the bioluminescence Flux endpoint of 2×10¹⁰ on two consecutive measurements, and/or ii) the above-mentioned general euthanasia criteria. For T-cell experiments in non-tumor bearing mice, mice received a single i.v. dose of 1×10⁷ NT or 1×10⁶ or 1×10⁷ CAR T-cells expressing GFP.ffLuc.

### Bioluminescence Imaging

Mice were injected i.p. with 150 mg/kg of D-luciferin 5-10 minutes before imaging, anesthetized with isoflurane, and imaged with a Xenogen IVIS-200 imaging system. The photons emitted from the luciferase-expressing tumor cells were quantified using Living Image software (Caliper Life Sciences). Mice were imaged once per week to track tumor burden, or 1-5 times per week to track T-cells.

### Angiosense Imaging

Mice where anesthetized with isoflurane and received a single i.v. dose of Angiosense 750 (PerkinElmer; NEV10011EX) via tail vain injection as recommended by the manufacturer. Administered Angiosense 750 was allowed to equilibrate in mice for 24 hours before imaging using a Xenogen IVIS-200 imaging system. Fluorescence signals were quantified using Living Image software (Caliper Life). Relative fluorescent units (RFU) where calculated by measuring tumor fluorescence subtracted from the background of mouse auto-fluorescence divided by tumor volume.

### Immunohistochemistry

Immunohistochemistry (IHC) was performed in collaboration with the SJCRH Veterinary Pathology Core Facility. After fixation, the tumor samples were embedded in paraffin and 8 μm sections were cut and mounted on slides. The sections were then processed and analyzed using immunohistochemistry with CD31 (Dianova, clone SZ31, cat. DIA 310). CD31 was only scored on those tumors+/−700 mm³ from the average tumor size in order to best control for the impact tumor size variation has on vascularization. CD31 levels where calculated by independent blind-scoring and images were acquired at the St. Jude Cell & Tissue Imaging Center using the Axio Scan Z.1.

### Statistical Analysis

All experiments were performed at least in triplicates. For comparison between two groups, two-tailed t-test was used. For comparisons of three or more groups, values were log transformed as needed and analyzed by ANOVA with Tukey's post-test. Survival was analyzed by Kaplan-Meier method and by the log-rank test. Bioluminescence imaging data were analyzed using either ANOVA, t-test, or area under the curve (AUC).

### Example 1. Expression of FN-EDB in Pediatric Solid Tumors and Brain

### Tumors Including Sarcoma

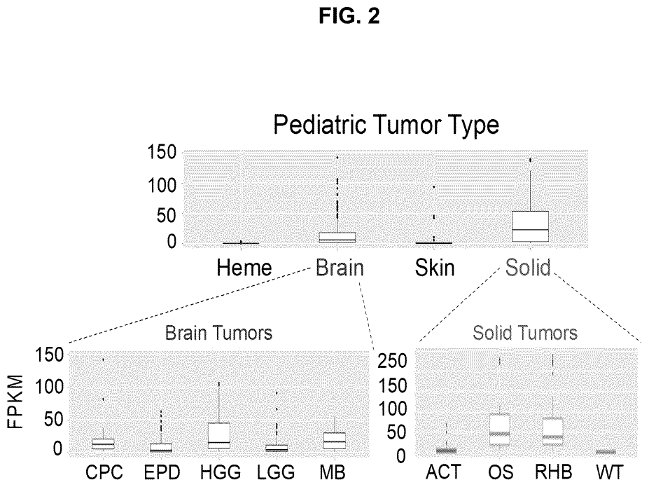

FN-EDB is expressed in a broad range of adult solid tumors, but little is known about expression in the pediatric solid tumors and brain tumors. The expression of FN-EDB was analyzed using the Pediatric Cancer Genome Project (PCGP) dataset, which includes 31 osteosarcoma (OS), 51 rhabdo-myosarcoma (RHB), 85 high grade glioma (HGG), 78 low grade gliomas (LGG) and 82 ependymomas (EPD). As shown in FIG. 2, FN-EDB is expressed in OS and RHB. Thus, this data demonstrates that FN-EDB is expressed in pediatric solid tumors as well as brain tumors.

### Example 2. Generation of EDB-CAR T-Cells

While FN-EDB is a secreted protein, it plays a major role in cellular adhesion,35 making it likely that FN-EDB-producing tumor cells (EDB+ tumor cells) can be targeted with EDB-CAR T-cells. To test this hypothesis a retroviral vector was designed encoding an EDB-specific CAR (EDB-CAR) using the EDB-specific scFv that has shown tumor specificity in imaging studies in humans (L19), a CD28hinge/transmembrane domain (CD28H/TM) and a CD28.ζ signaling domain (FIG. 3A). EDB-CAR T-cells were generated by retroviral transduction of CD3/CD28-activated T-cells in the presence of IL7 (10 ng/ml) and IL15 (10 ng/ml). CAR expression was readily detected on transduced T-cells by FACS analysis (FIG. 3B). In brief, cells were harvested, washed, and stained with isotype control or a Fab antibody to detect the CAR. Cells were analyzed on a BD Canto cell analyzer. Retroviral T-cell transduction was confirmed using anti-CD19. EDB-CAR T-cells contained a mixture of CD4- and CD8-positive T-cell, and further T-cell subset analysis revealed the presence of naïve, central memory, effector memory, and terminally differentiated memory T-cells. To confirm the specificity of EDB-CAR T-cells, CAR T-cells and non-transduced T-cells (NT T-cells) were incubated with increasing concentration of recombinant EDB protein (purchased from Abcam) (0 ng, 0.5 ng, 1 ng, or 10 ng). Only, EDB-CAR T-cells produced significant amount IFNγ, indicating specific EDB-CAR T-cell activation (FIG. 3C).

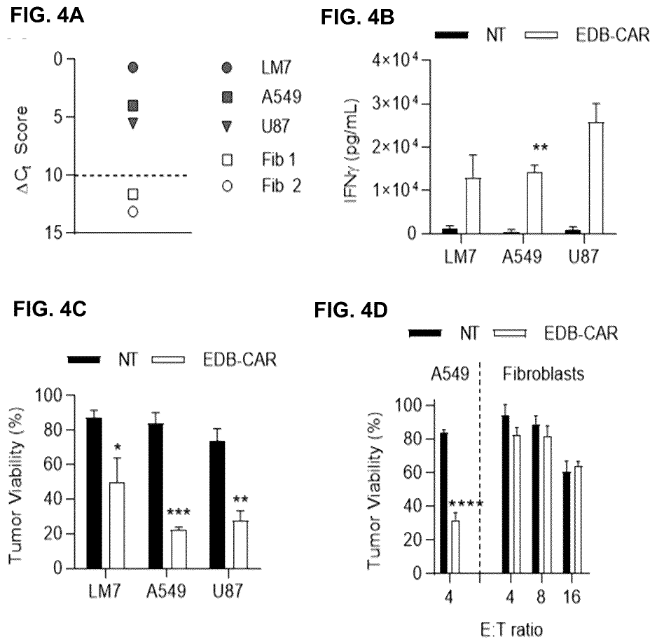

To evaluate if EDB-CAR T-cells recognize and kill EDB+ tumor cells, three EDB+ tumor cells (LM7: osteosarcoma, A549: lung cancer, U87: high grade glioma) were used (FIG. 4A). To measure IFNγ secretion, 5×10⁵ tumor cells and 1×10⁶ T-cells were cocultured in wells of a 24 well tissue culture plate. After 24 hours media was harvested and IFNγ production was determined by ELISA. EDB-CAR T-cells readily recognized EDB+ tumor cells as judged by IFNγ secretion (FIG. 4B) in contrast to NT T-cells. To determine the cytolytic activity of CAR and NT T-cells, standard MTS (3-(4,5-dimethylthiazol-2-yl)-5-(3-carboxymethoxyphenyl)-2-(4-sulfophenyl)-2H-tetrazolium) assays were performed. Briefly, tumor cells and T-cells were plated in wells of 96 well plates at a T-cell to tumor cell ratio of 8:1. After 3 days T-cells were gently removed and viable tumor cells were quantified using the MTS reagent according to the manufacturer's instructions. EDB-CAR T-cells killed EDB+ tumor cells in contrast to NT T-cells (FIG. 4C). In contrast, EDB-CAR T-cells had no cytolytic activity against primary human fibroblasts even at high effector to target (E:T) ratios of 16:1 (FIG. 4D).

### Example 3. EDB-CAR T-Cells have Antitumor Activity In Vivo

In this example, three studies were performed to determine if EDB-CAR T-cells have antitumor activity in vivo.

In the first experiment, 2×10⁶ A549 cells were injected subcutaneously into immunodeficient NSG mice, and on day 20, mice received a single intravenous injection of 3×10⁵ T-cells (FIG. 5A), a cell dose which 10× fold less than is normally used in preclinical models. Mice received EDB-CAR T-cells, EphA2-CAR (as a positive control for a CAR T-cells that work well at T-cell doses of 3×10⁶) or NT T-cells. Tumor growth was measured by serial caliper measurements. While NT- and EphA2-CAR T-cells had no or very limited antitumor activity, 4 out of 5 mice treated with EDB-CAR T-cells had tumor regression (FIG. 5B). Of these, two mice required euthanasia due to skin necrosis covering their small tumor, and two mice achieved a long-lasting CR (>100 days post CAR T-cell injection). The tumors of euthanized mice were examined for the presence to blood vessels (CD31 staining) and compared to untreated tumors and tumors treated with EphA2-CAR T-cells (FIG. 5C, FIG. 5D). EDB-CAR treated tumors had a reduced vessel density as judged by CD31 staining. This data demonstrates that EDB-CAR T-cells have antitumor activity in subcutaneous tumor model in vivo.

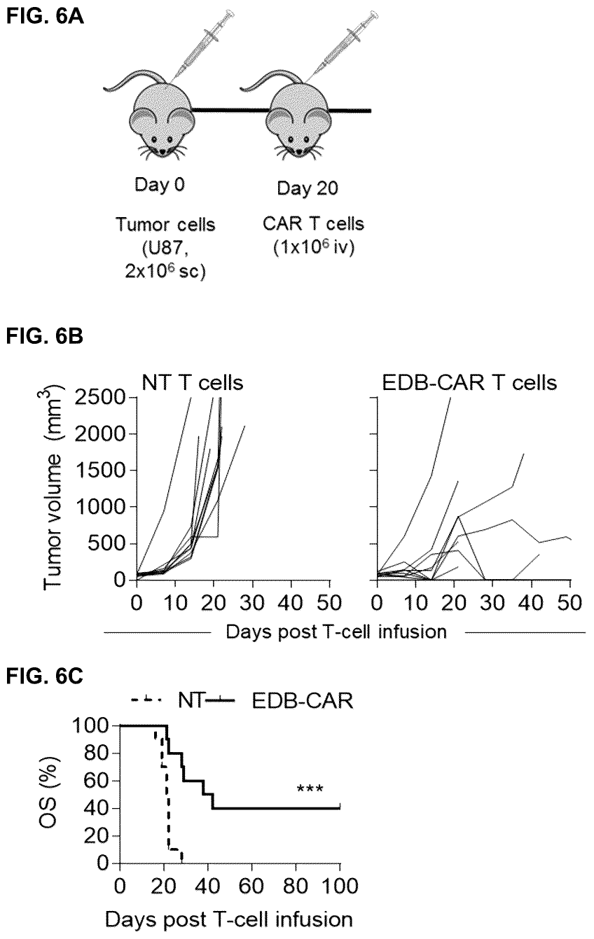

In the second experiment, 2×10⁶ U87 cells were injected subcutaneously into immunodeficient NSG mice, and on day 7, mice received a single intravenous injection of 1×10⁶ EDB-CAR or NT T-cells (n=5 per group). Tumor growth was measured by serial caliper measurements. EDB-CAR T-cells had potent antitumor activity (FIG. 6A) that translated into a significant survival advantage (FIG. 6B).

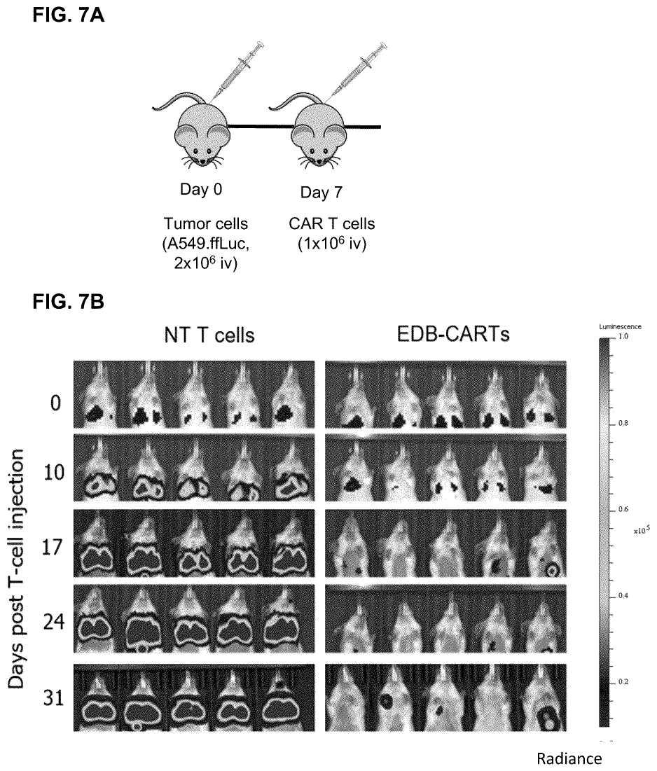

In the third experiment, 2×10⁶ GFP-ffluc-expressing A549 cells were injected intravenously into immunodeficient NSG mice, and on day 7 mice received iv 1×10⁶ NT or EDB-CAR T-cells (FIG. 7A). Tumor growth was followed by serial bioluminescence imaging using an IVIS imager and luciferase as a substrate. EDB-CAR T-cells had significant antitumor activity as judged by bioluminescence imaging in comparison to NT T-cells with a follow up of 31 days post T-cell injection (FIG. 7B).

Thus, these studies indicate that EDB-CAR T-cells have potent antitumor activity. Of note, toxicity was not observed as judged by the lack of weight loss, change in appearance, and/or activity. Since human and murine EDB is 100% identical, these studies thus highlight an encouraging safety profile of EDB-CAR T-cells.

The L19-based CAR not only recognized and killed L19-CAR T-cells EDB+ tumor cells in cell culture experiments, but it surprisingly also had potent antitumor activity in two immunodeficient mouse models, demonstrating that EDB-CAR T-cells do not need other immune cells in vivo to exert their antitumor activity.

### Example 4. Indirect Targeting of Tumor Cells

FIG. 8A depicts an exemplary scheme of a vector that encodes an EDB-specific synNotch receptor, which will induce the expression of a TAA-specific CAR or BiTE molecule. FIG. 8C depicts a vector for combined indirect and direct targeting of tumor cells with EDB-CAR T-cells, wherein T-cell activation via the EDB-CAR.synNotch interacting with the EDB located on the tumor stroma induces expression of a second CAR (e.g., TAA-specific CAR) or BiTE that directly targets the tumor cells.

A synNotch inducible lentiviral vector is generated to link the expression of a second CAR or BiTE molecule to the recognition of EDB by T-cells expressing an EDB-specific SynNotch receptor; see FIG. 8B). T-cell expressing the synNotch receptor alone will not be able to directly kill the tumor cells, even those expressing the cognate antigen. Thus, this synNotch receptor construct is used to only induce the expression of the second CAR or BiTE in the tumor ECM in which EDB in present.

Next, a second lentiviral vector is generated that encodes a constitutive EDB-CAR in addition to the synNotch receptor (i.e., EDB-notch-CAR; see FIG. 8C).

An example of how the indirect approach will be tested in vitro and in vivo is provided below. A lentiviral vector will be generated that encodes the EDB-specific SynNotch receptor and a bispecific T-cell engager BiTE) that recognizes the TAA EphA2 (EA2-ENG).

In vitro analysis. Three T-cell populations: i) EphA2-specific BiTE-Notch T-cells) (EA2-ENG.EDB-Notch T-cells), ii) EA2-ENG T-cells, and iii) NT T-cells are tested. The ability of each cell population to recognize EDB+ target cells and secrete EA2-ENG will be assessed. The same tumor cells U87, A549, LM7) and assays that were used in the in vitro experiment shown in FIG. 3 (Example 2) for EDB-CAR T-cells will be used; in addition, an ELISA to determine the concentration of EA2-ENG will be used.

In vivo analysis. The same experimental set up as shown for the analysis of EDB-CAR T-cells in vivo (FIGS. 4 to 7; Example 3) will be used. Immunodeficient NSG mice will be injected subcutaneously with U87 or A549 cells and will received a single dose of EA2-ENG.EDB-Notch T-cells, EA2-ENG T-cells, or NT T-cells. Tumor cell growth will be measured by caliper measurement. The anti-vascular activity of T-cells will be determined by IHC for CD31. The antitumor activity of EA2-ENG.EDB-Notch T-cells in the systemic tumor cell model in which A549 cells are injected intravenously as shown in FIG. 7 will also be assessed.

For the BiTE molecule approach, a lentiviral vector in which the inducible, tumor-specific molecule is an EA2-specific CAR will be constructed an evaluated as described above, wherein the only difference is that CAR expression will be measured by FACS analysis and not EA2-ENG production by ELISA. In addition, the indirect approach for other TAAs for which CARs and/or engager molecules are available (e.g., HER2, GD2, IL13Rα2, or B7-H3) will be tested.

### Example 5. Additional EDB-CAR T-Cells

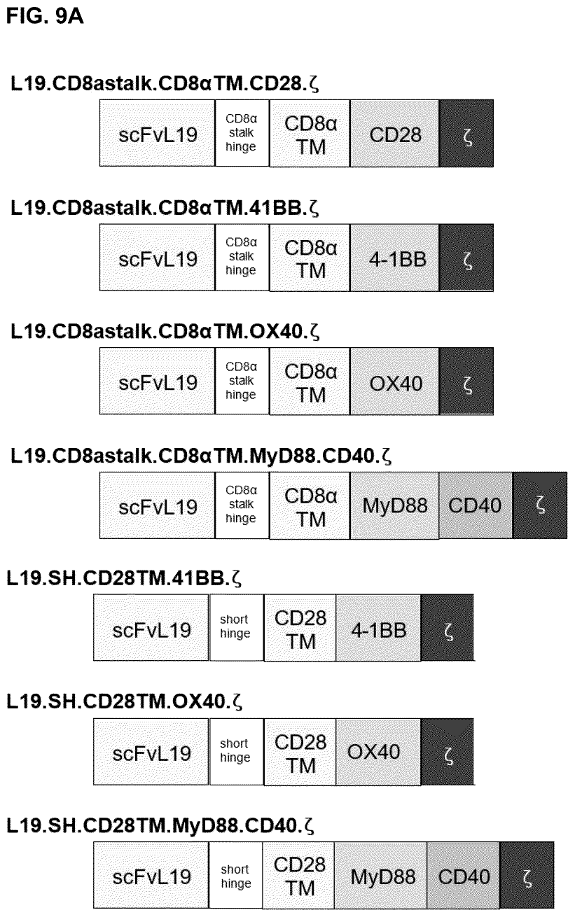

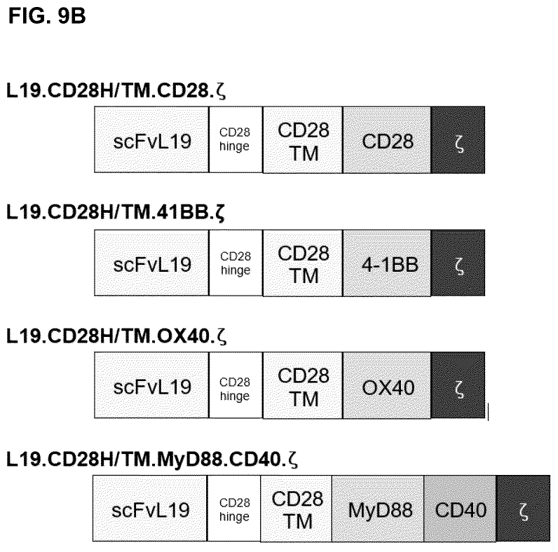

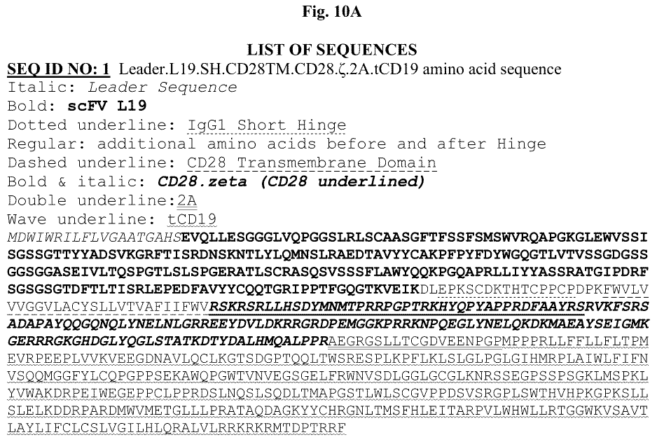

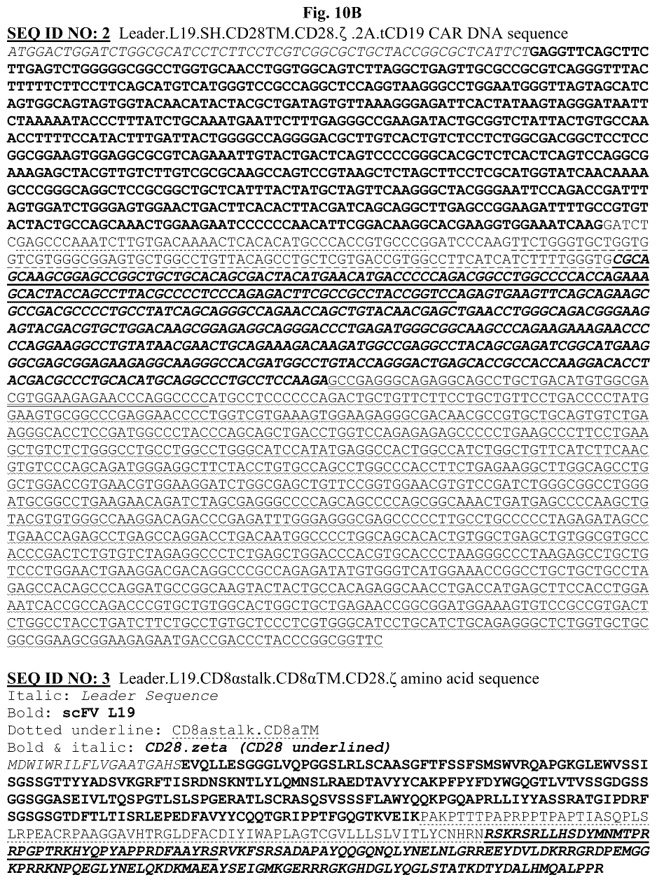

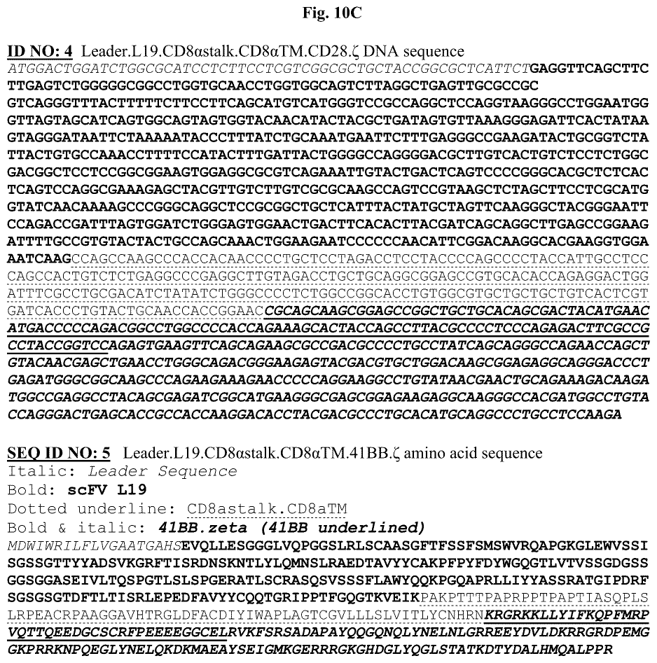

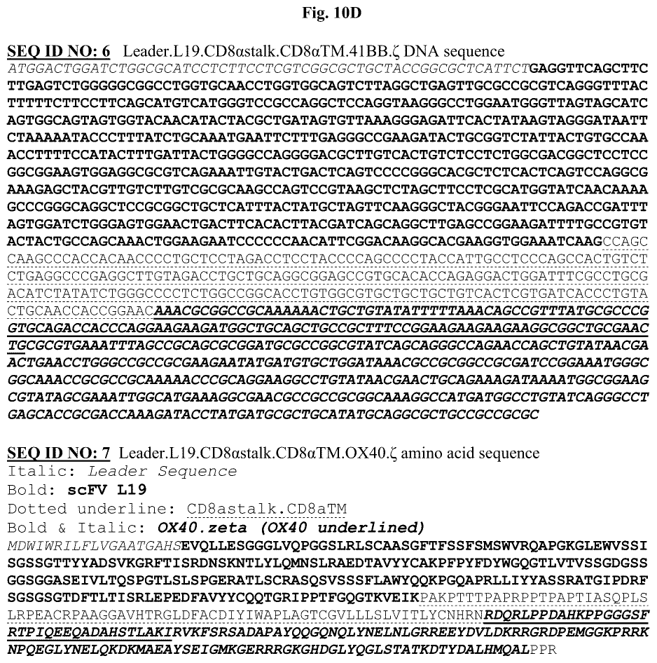

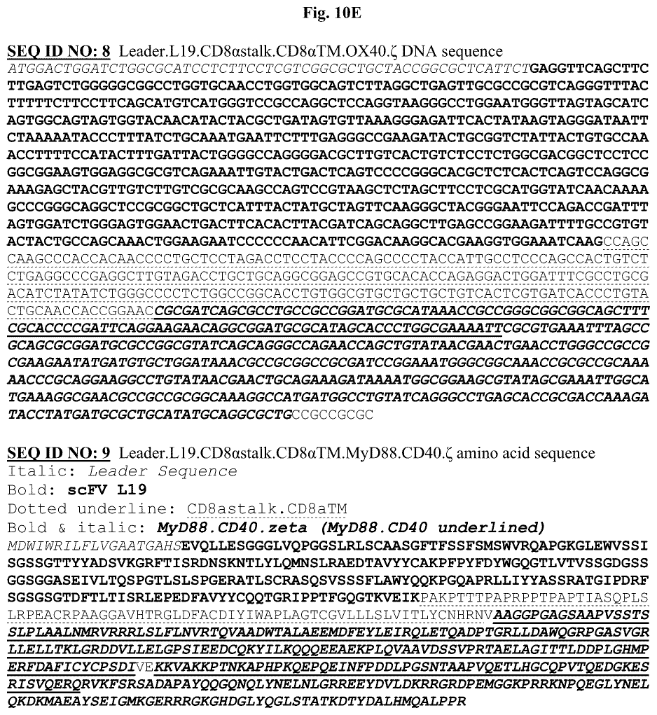

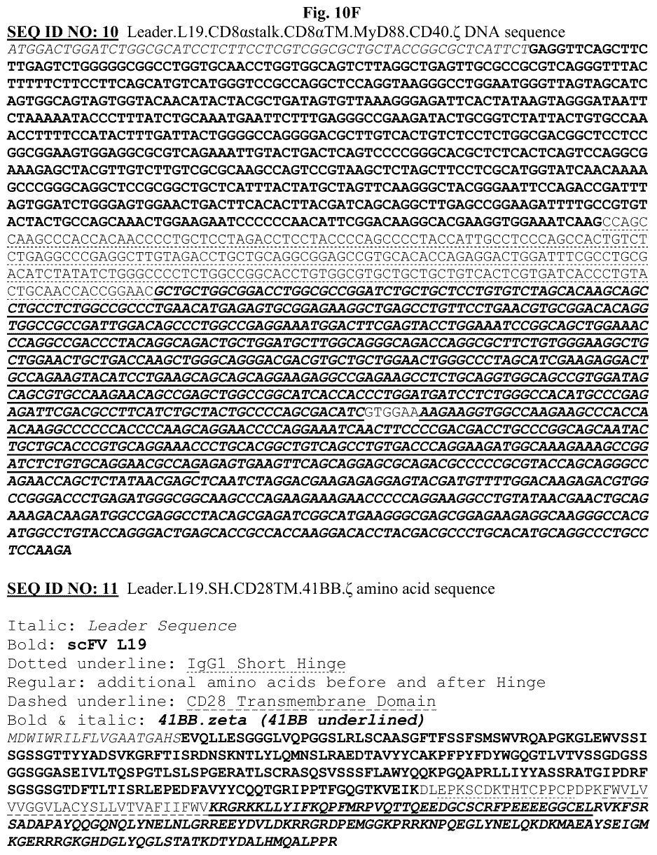

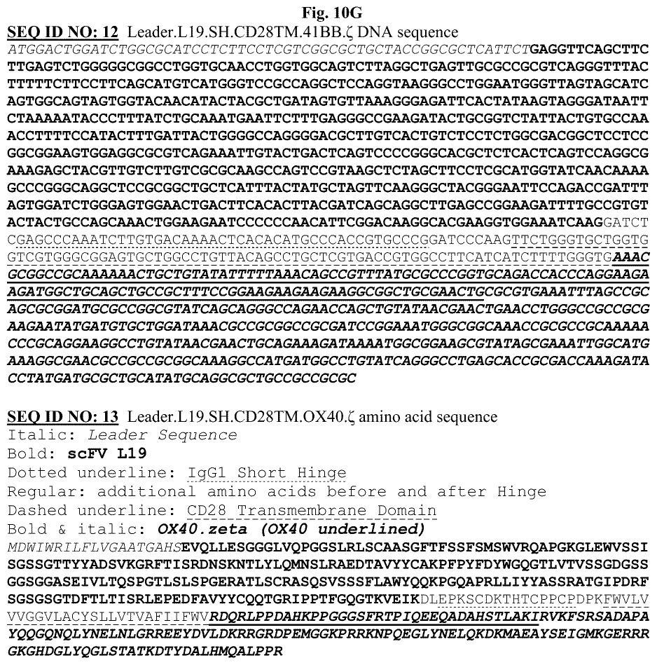

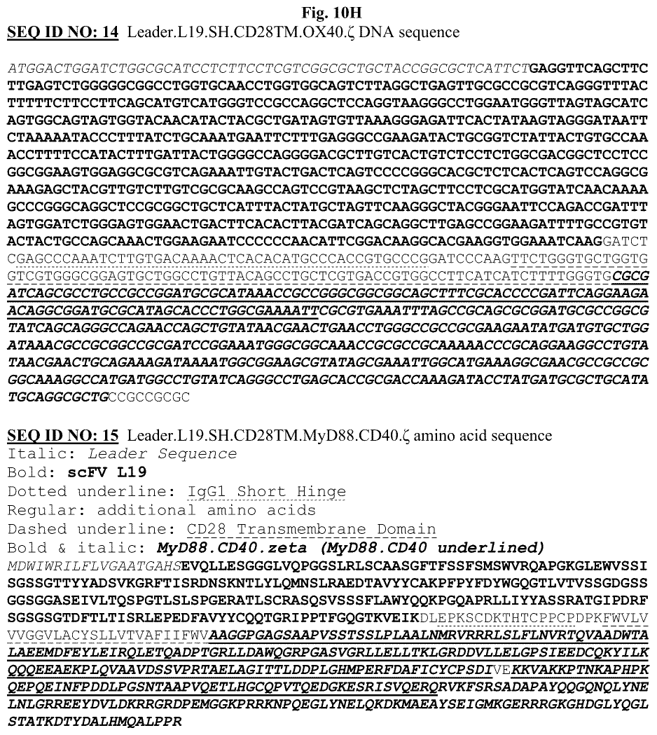

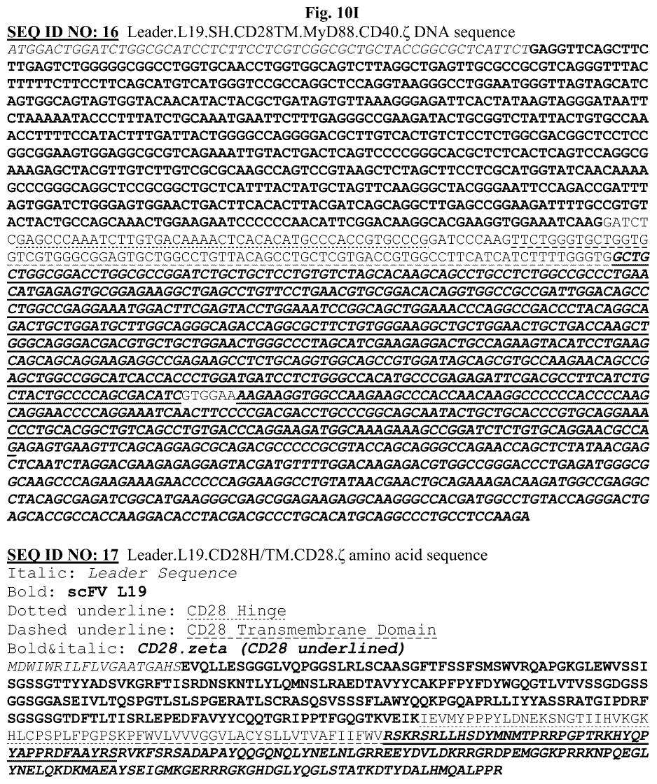

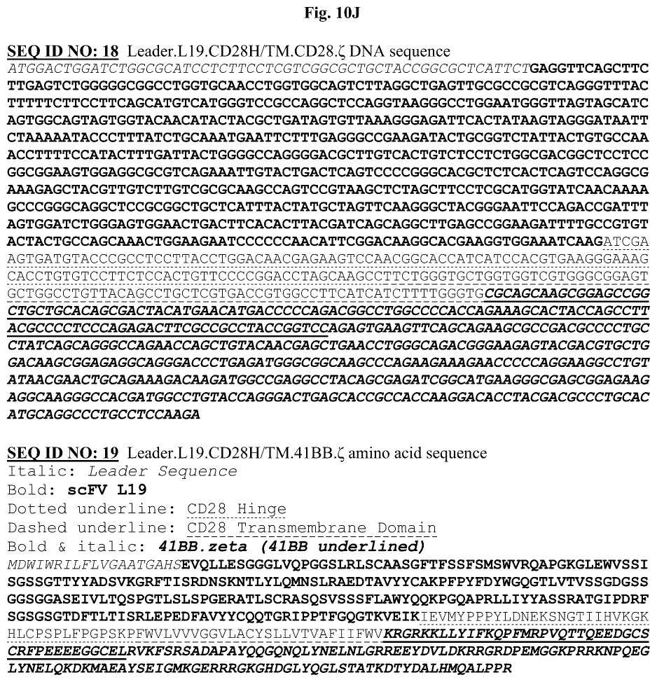

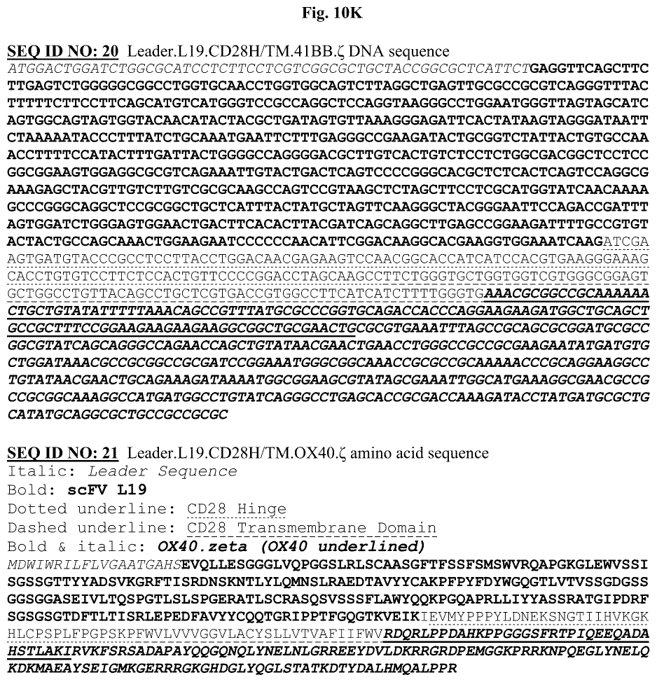

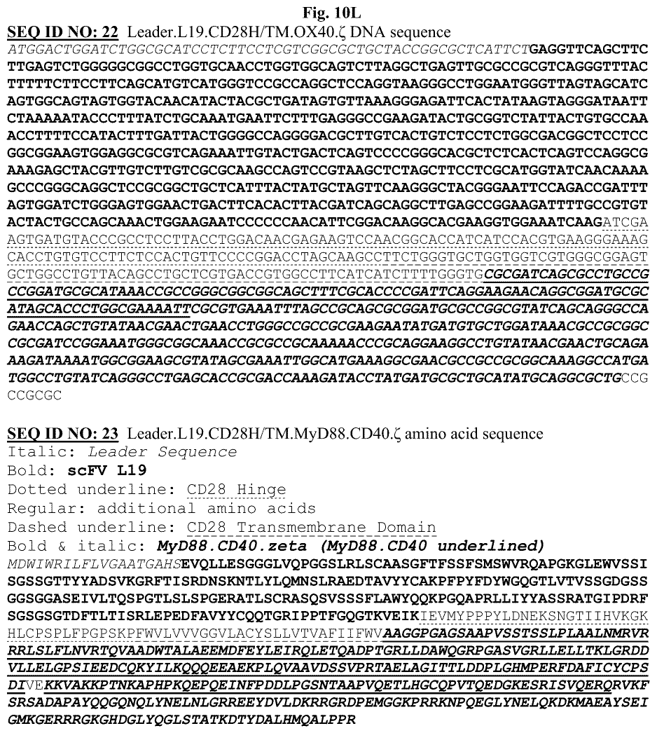

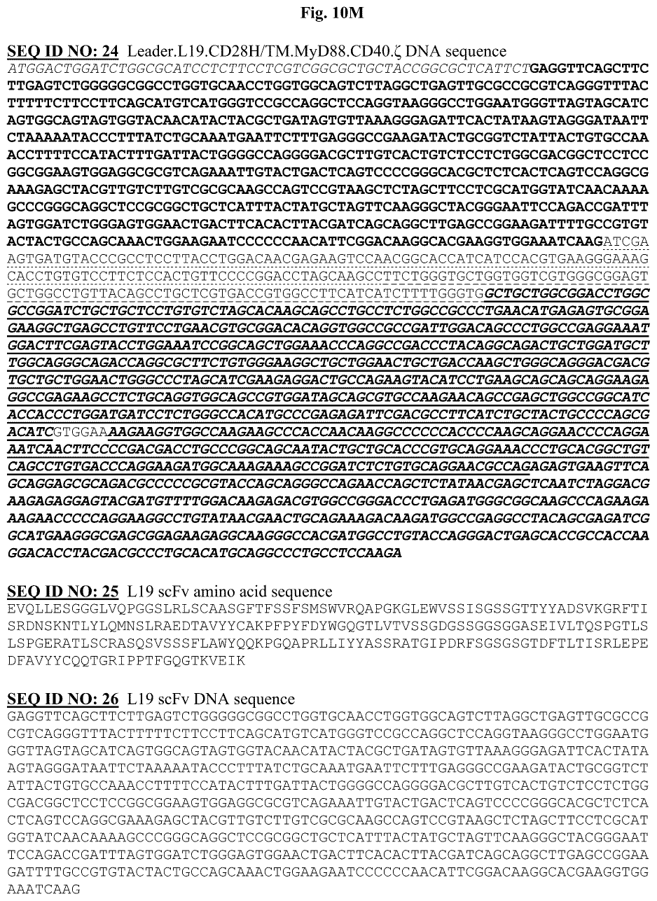

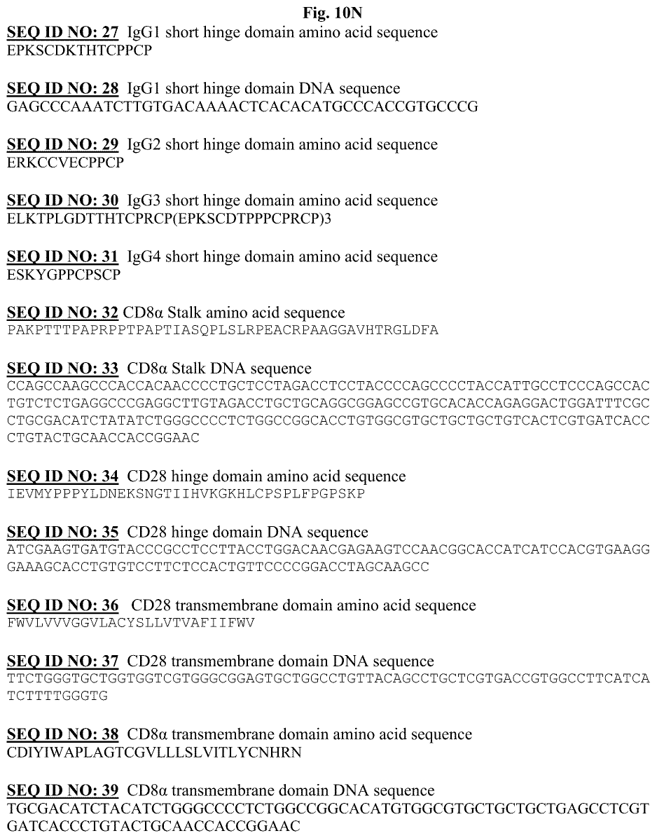

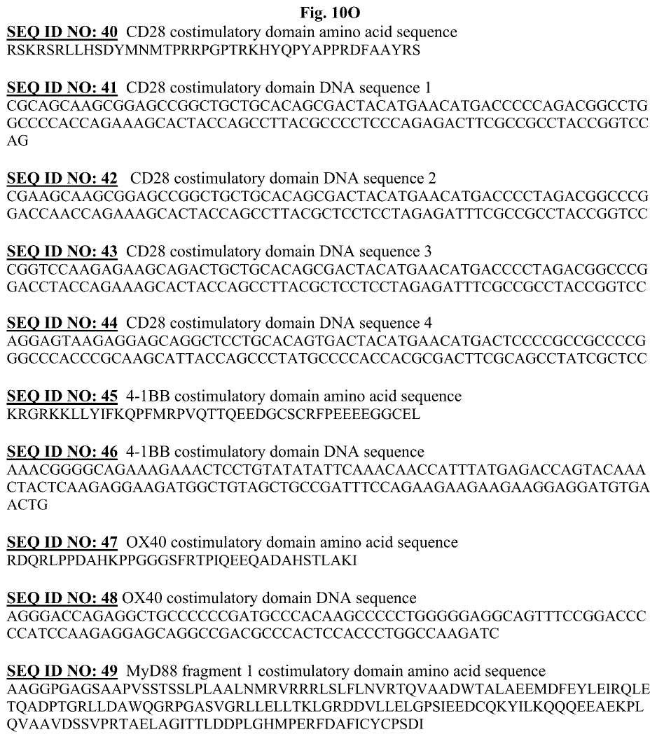

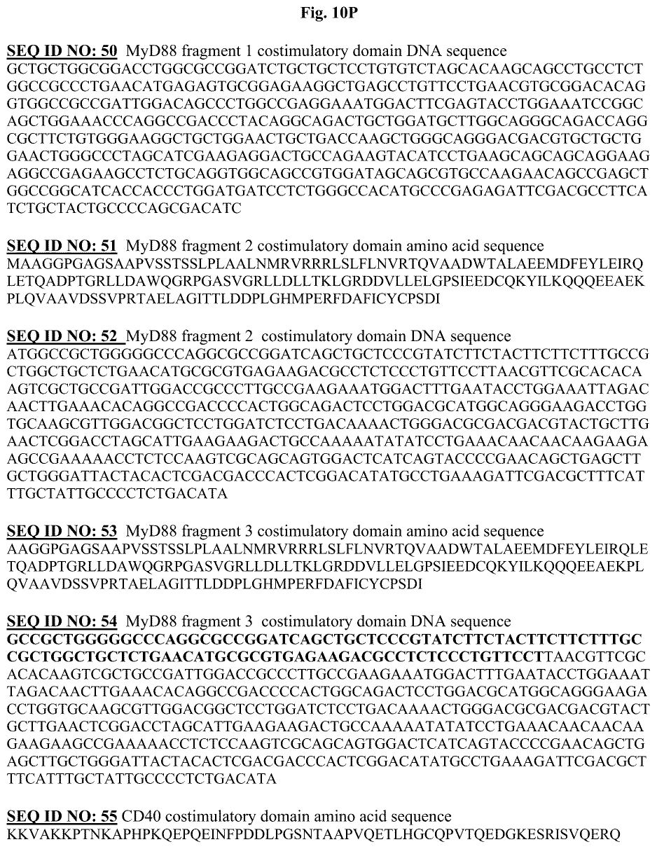

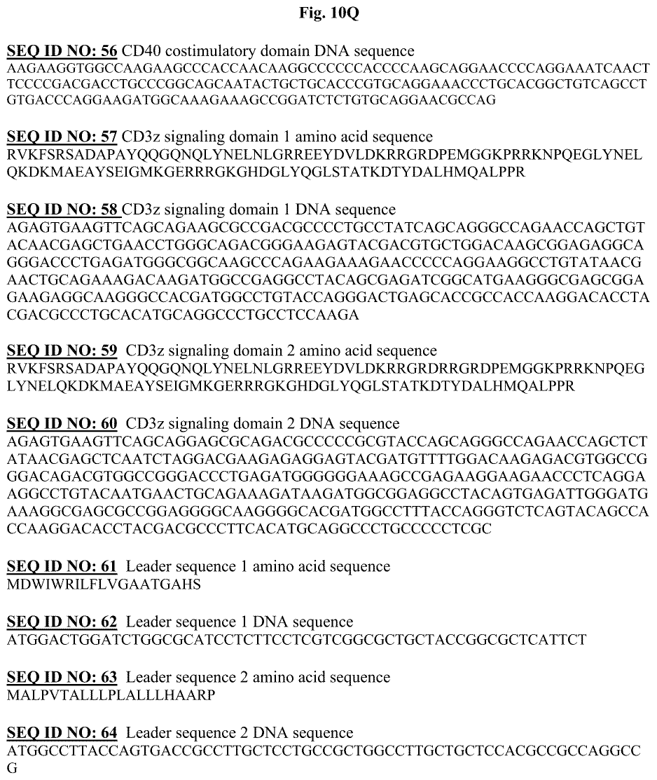

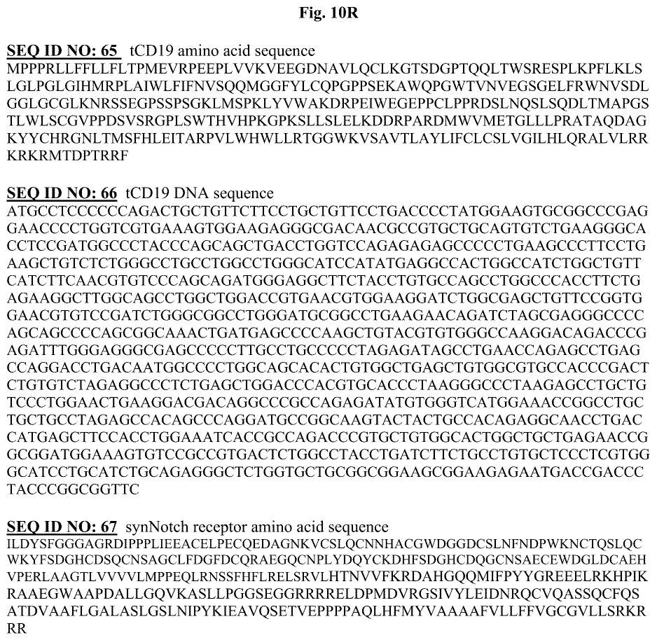

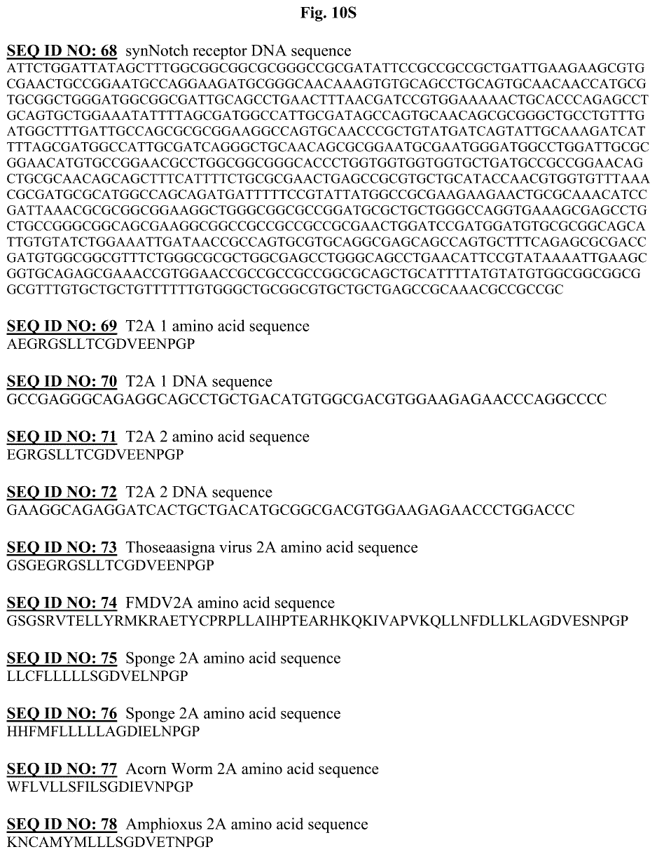

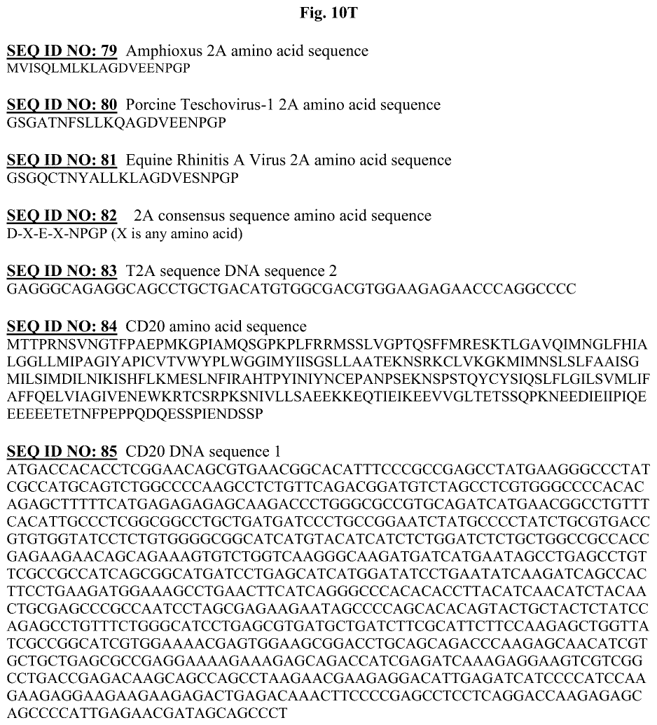

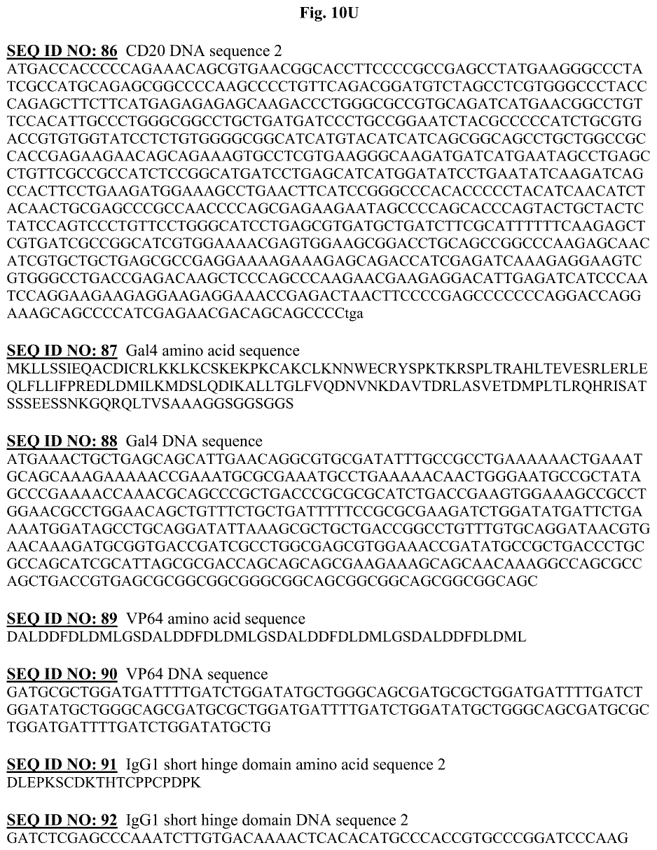

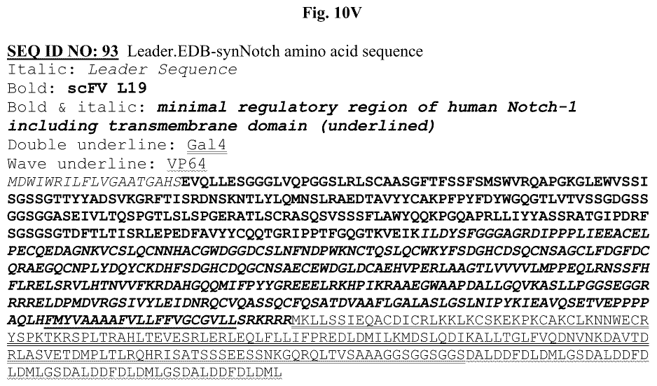

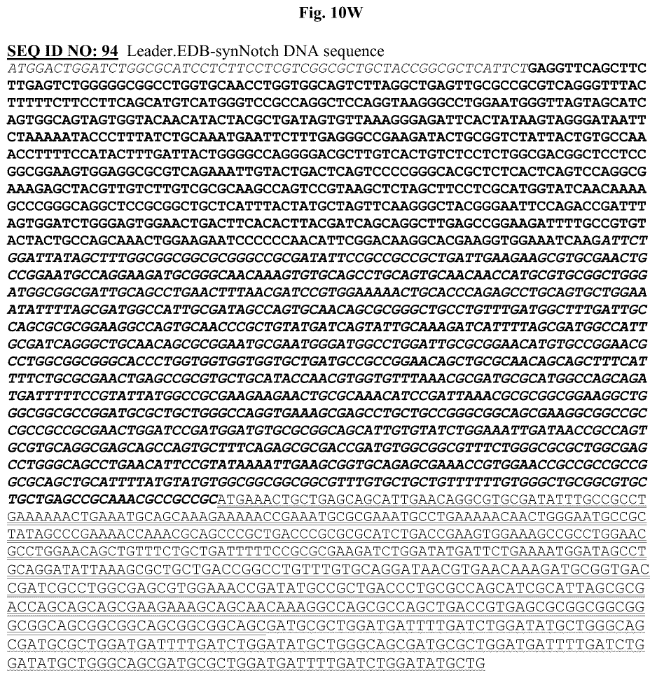

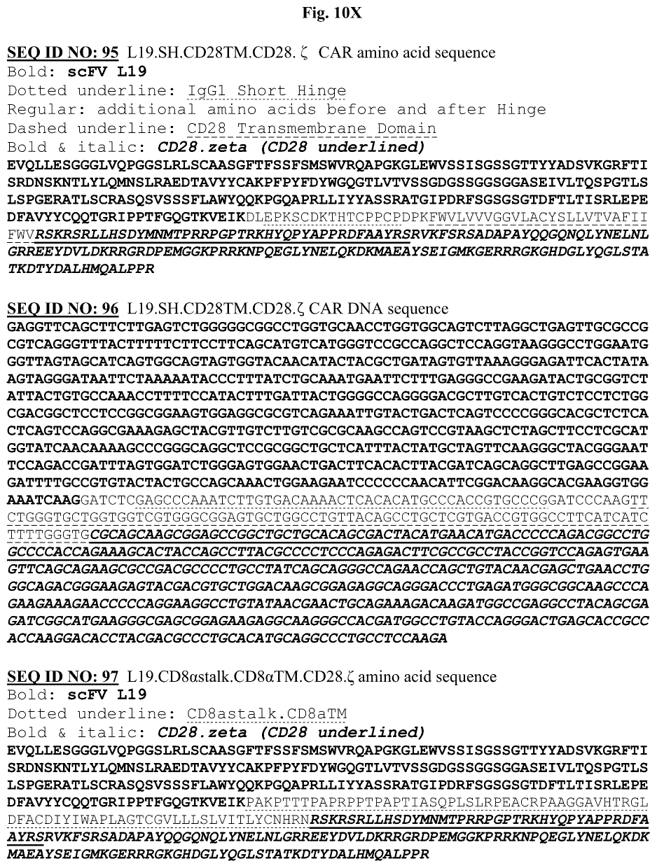

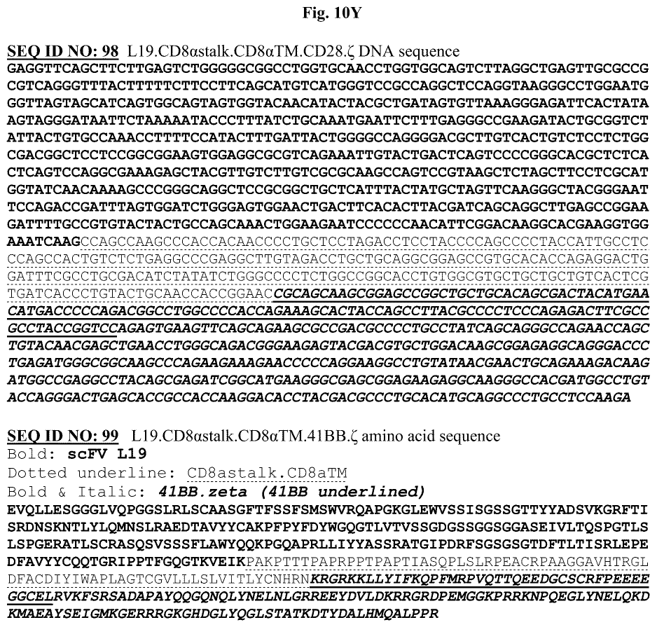

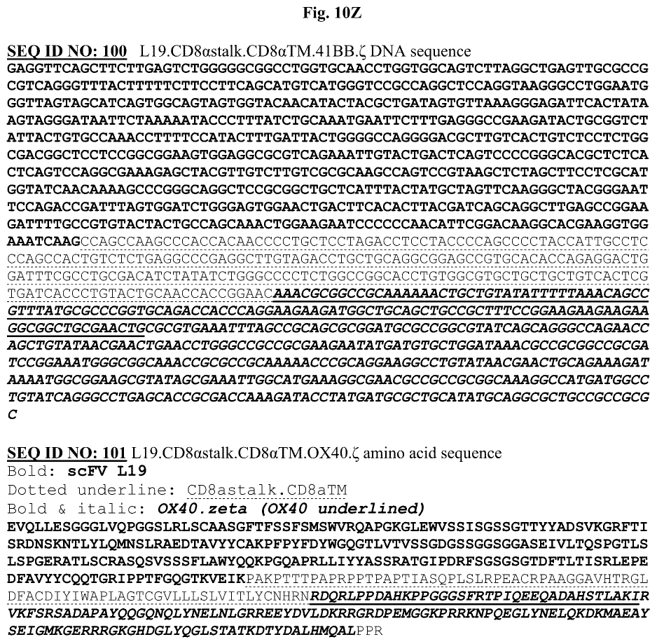

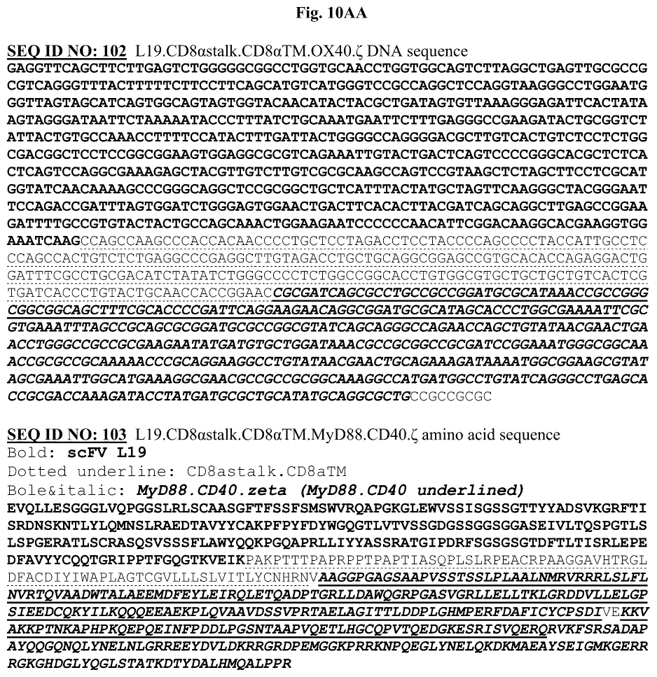

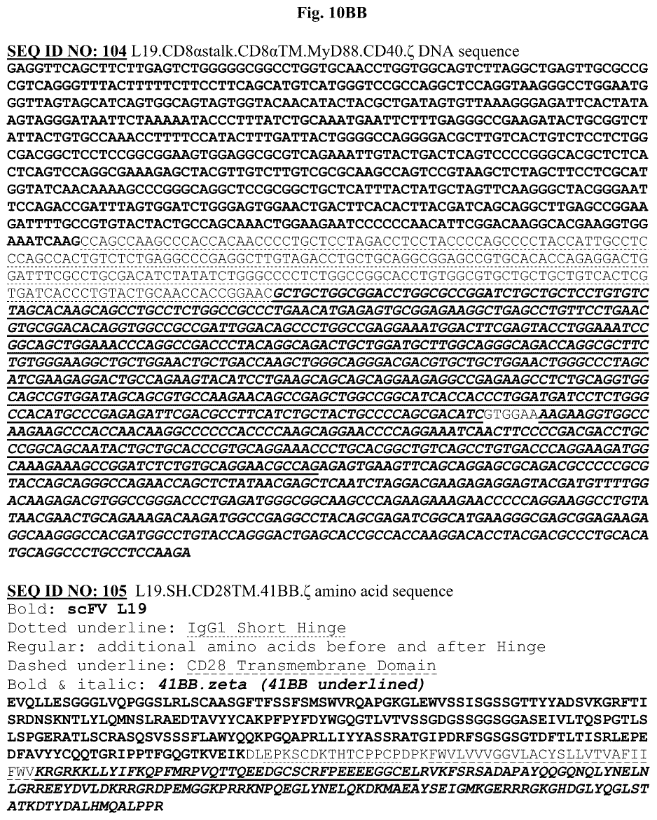

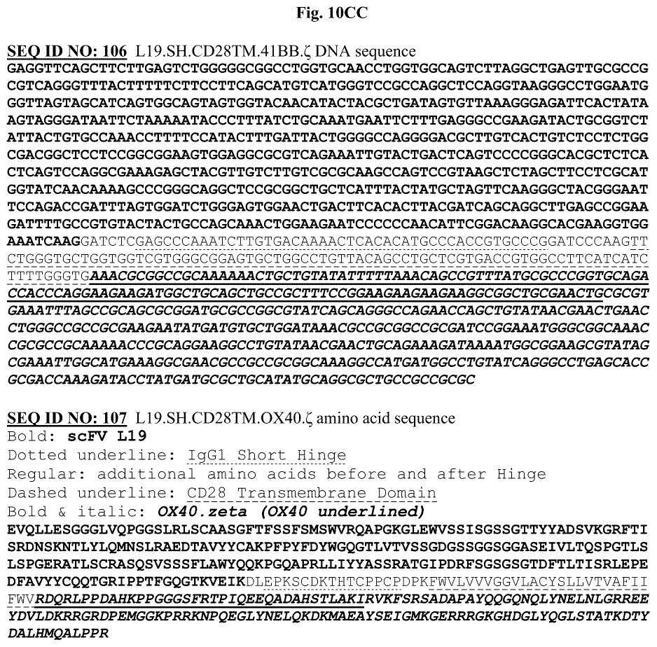

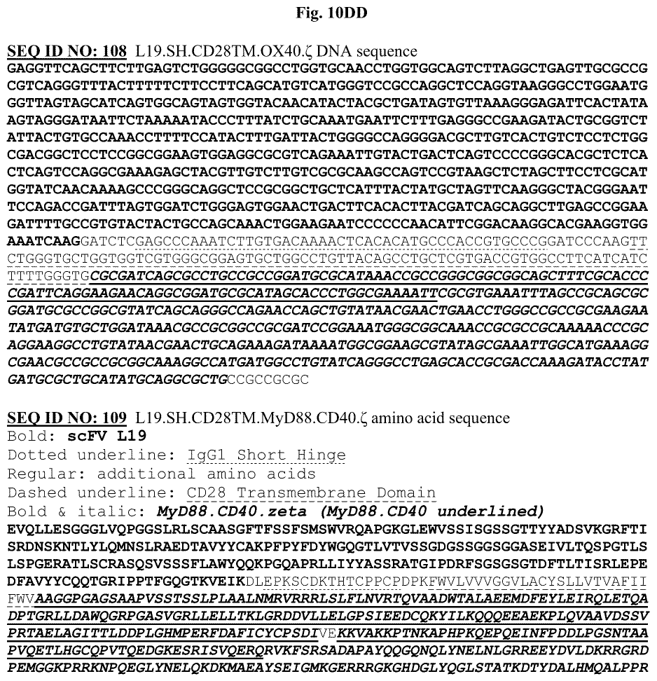

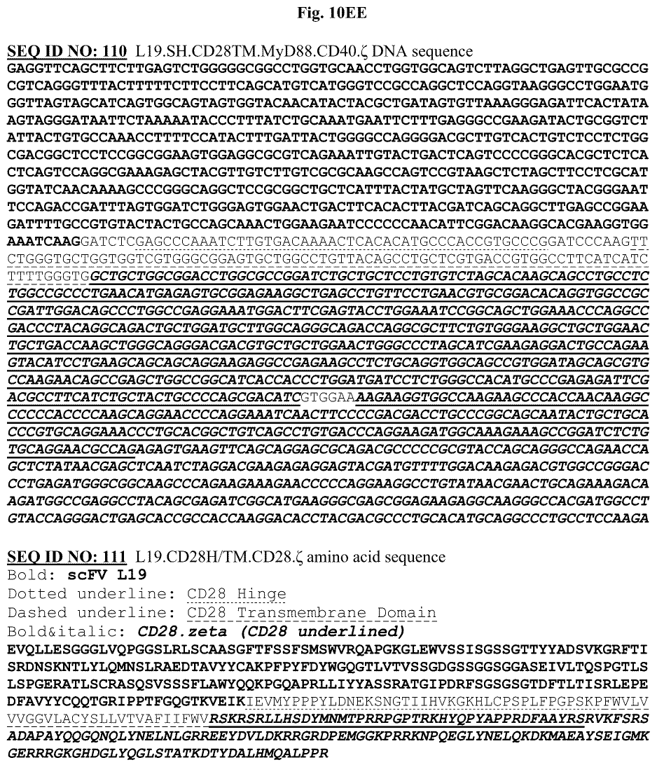

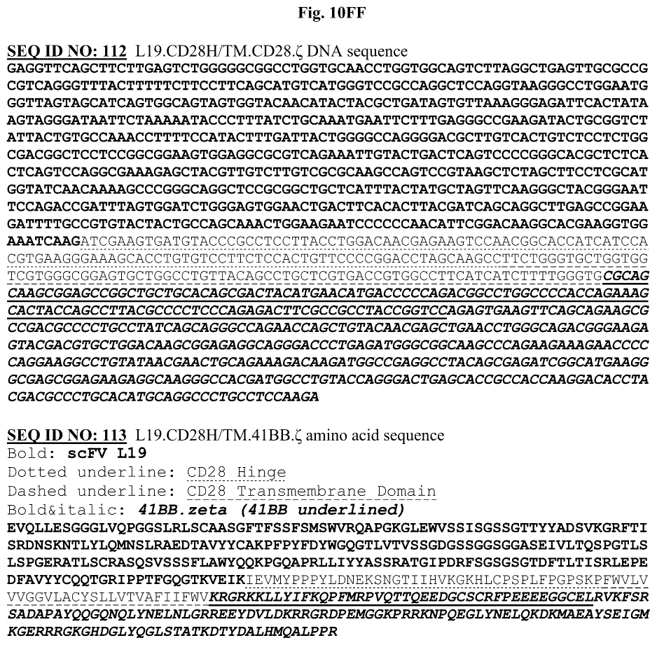

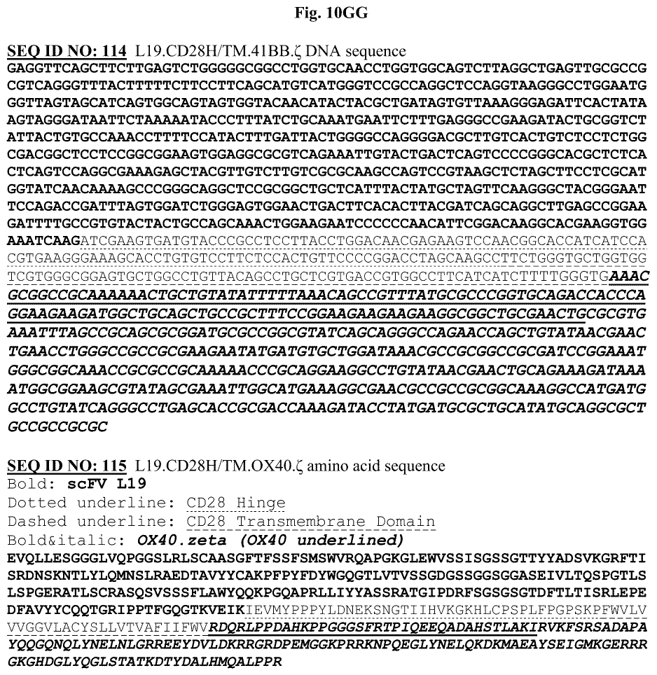

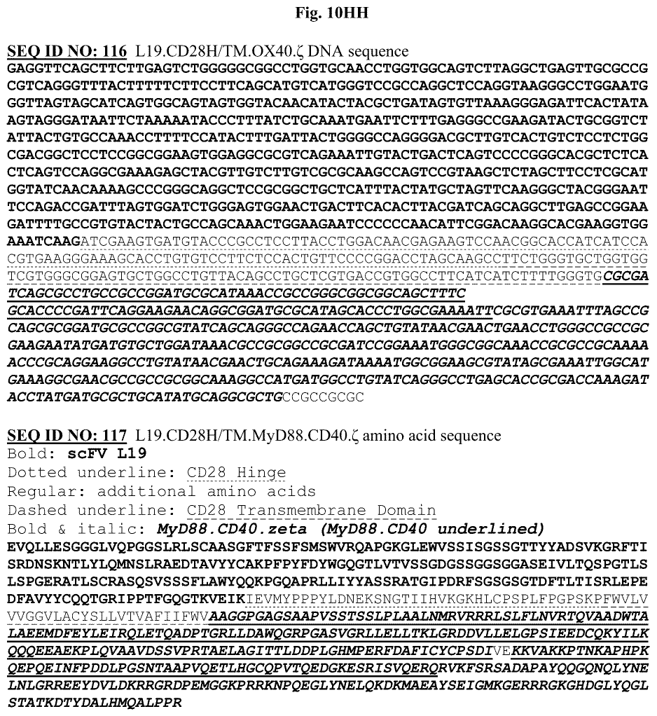

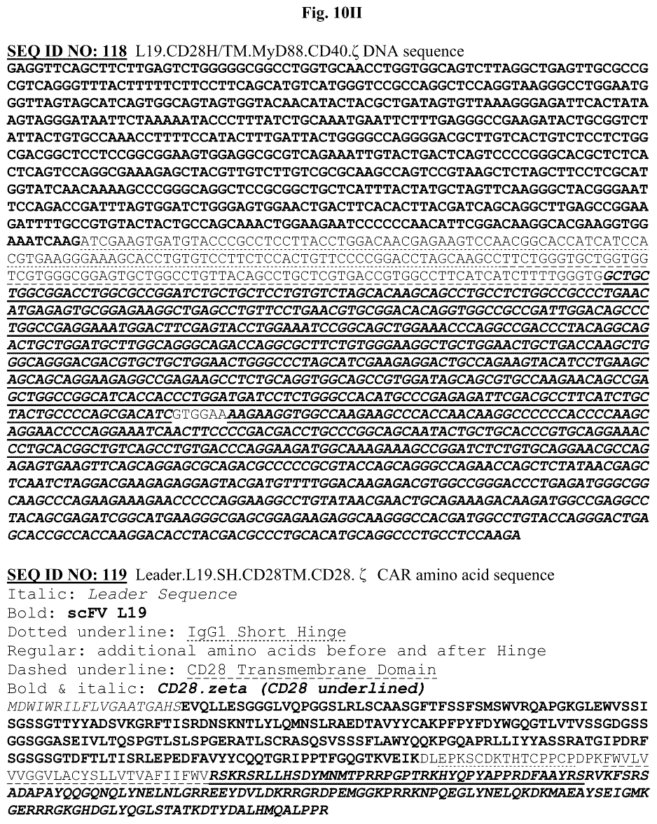

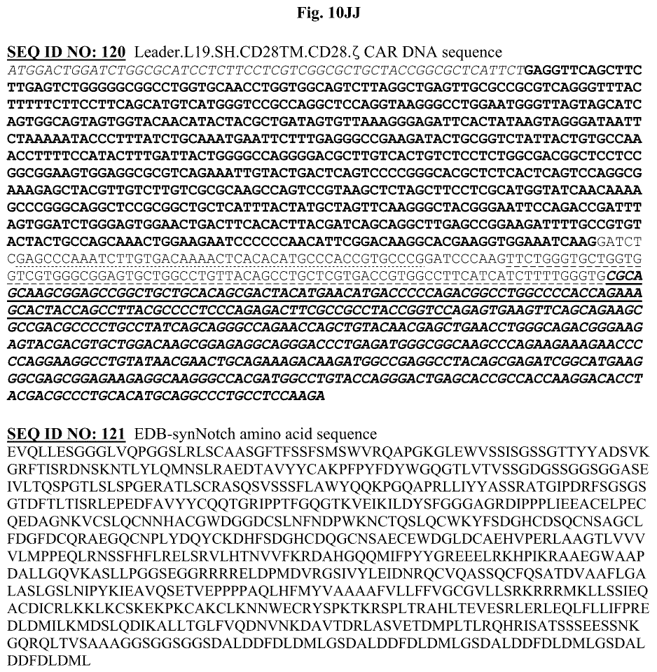

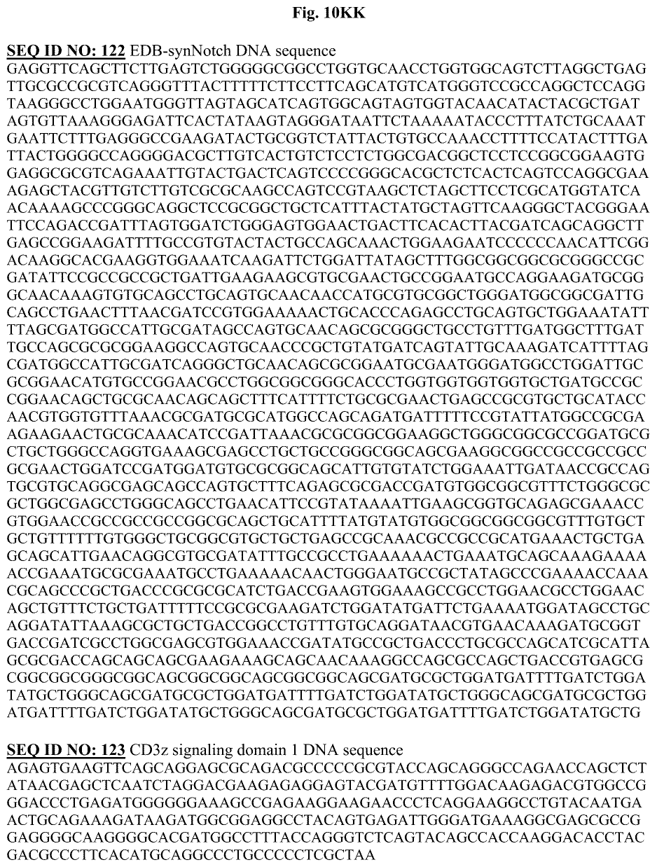

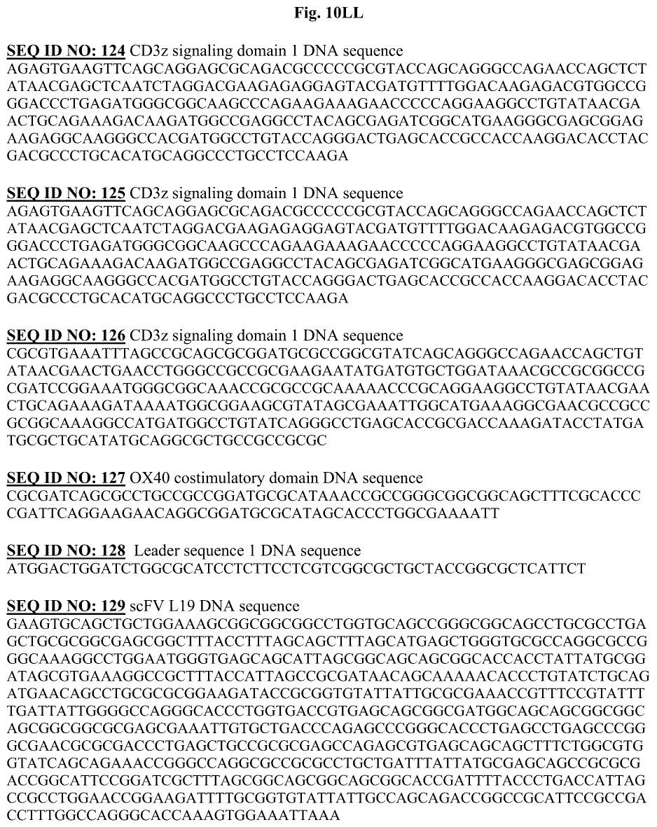

Additional retroviral constructs (FIG. 9) are generated by cloning the EDB-specific scFv into CAR expression cassettes, such as but not limited to the expression cassettes discussed in Krenciute et al., Cancer Immunology Research, (2017) 5:571-81; Yi et al., Mol. Ther. Methods Clin. Dev. (2018) 9:70-80; or Mata et al., Cancer Discovery (2017) 7:1306-19, each of which are incorporated herein by reference in their entirety. RD114-pseudotyped retroviral vectors are generated by standard methods and EDB-CAR T-cells are generated as outlined in Example 2.

### REFERENCES

1. Maude S L, Laetsch T W, Buechner J, et al. Tisagenlecleucel in Children and Young Adults with β-Cell Lymphoblastic Leukemia. The New England journal of medicine 2018; 378:439-48.
2. Park J H, Riviere I, Gonen M, et al. Long-Term Follow-up of CD19 CAR Therapy in Acute Lymphoblastic Leukemia. The New England journal of medicine 2018; 378:449-59.
3. Kochenderfer J N, Dudley M E, Kassim S H, et al. Chemotherapy-refractory diffuse large β-cell lymphoma and indolent β-cell malignancies can be effectively treated with autologous T cells expressing an anti-CD19 chimeric antigen receptor. Journal of clinical oncology: official journal of the American Society of Clinical Oncology 2015; 33:540-9.
4. Gardner R A, Finney O, Annesley C, et al. Intent-to-treat leukemia remission by CD19 CAR T cells of defined formulation and dose in children and young adults. Blood 2017; 129:3322-31.
5. Ahmed N, Brawley V S, Hegde M, et al. Human Epidermal Growth Factor Receptor 2 (HER2)-Specific Chimeric Antigen Receptor-Modified T Cells for the Immunotherapy of HER2-Positive Sarcoma. Journal of clinical oncology: official journal of the American Society of Clinical Oncology 2015; 33:1688-96.
6. Heczey A, Louis C U, Savoldo B, et al. CAR T Cells Administered in Combination with Lymphodepletion and PD-1 Inhibition to Patients with Neuroblastoma. Molecular therapy: the journal of the American Society of Gene Therapy 2017; 25:2214-24.
7. Zhang C, Wang Z, Yang Z, et al. Phase I Escalating-Dose Trial of CAR-T Therapy Targeting CEA (+) Metastatic Colorectal Cancers. Molecular therapy: the journal of the American Society of Gene Therapy 2017; 25:1248-58.
8. Thistlethwaite F C, Gilham D E, Guest R D, et al. The clinical efficacy of first-generation carcinoembryonic antigen (CEACAM5)-specific CAR T cells is limited by poor persistence and transient pre-conditioning-dependent respiratory toxicity. Cancer immunology, immunotherapy: CII 2017; 66:1425-36.
9. Knochelmann H M, Smith A S, Dwyer C J, Wyatt M M, Mehrotra S, Paulos C M. CAR T Cells in Solid Tumors: Blueprints for Building Effective Therapies. Frontiers in immunology 2018; 9:1740.
10. Long K B, Young R M, Boesteanu A C, et al. CAR T Cell Therapy of Non-hematopoietic Malignancies: Detours on the Road to Clinical Success. Frontiers in immunology 2018; 9:2740.
11. O'Rourke D M, Nasrallah M P, Desai A, et al. A single dose of peripherally infused EGFRvIII-directed CAR T cells mediates antigen loss and induces adaptive resistance in patients with recurrent glioblastoma. Science translational medicine 2017; 9.
12. Sauer S, Erba P A, Petrini M, et al. Expression of the oncofetal ED-B-containing fibronectin isoform in hematologic tumors enables ED-B-targeted 131I-L19SIP radioimmunotherapy in Hodgkin lymphoma patients. Blood 2009; 113:2265-74.
13. Santimaria M, Moscatelli G, Viale G L, et al. Immunoscintigraphic detection of the ED-B domain of fibronectin, a marker of angiogenesis, in patients with cancer. Clinical cancer research: an official journal of the American Association for Cancer Research 2003; 9:571-9.
14. Fukuda T, Yoshida N, Kataoka Y, et al. Mice lacking the EDB segment of fibronectin develop normally but exhibit reduced cell growth and fibronectin matrix assembly in vitro. Cancer research 2002; 62:5603-10.
15. Velasquez M P, Torres D, Iwahori K, et al. T cells expressing CD19-specific Engager Molecules for the Immunotherapy of CD19-positive Malignancies. Scientific reports 2016; 6:27130.
16. Iwahori K, Kakarla S, Velasquez M P, et al. Engager T cells: a new class of antigen-specific T cells that redirect bystander T cells. Molecular therapy: the journal of the American Society of Gene Therapy 2015; 23:171-8.
17. Skapek S X, Ferrari A, Gupta A A, et al. Rhabdomyosarcoma. Nat Rev Dis Primers 2019; 5:1.
18. Grunewald T G P, Cidre-Aranaz F, Surdez D, et al. Ewing sarcoma. Nat Rev Dis Primers 2018; 4:5.
19. Saraf A J, Fenger J M, Roberts R D. Osteosarcoma: Accelerating Progress Makes for a Hopeful Future. Front Oncol 2018; 8:4.
20. Dotti G, Gottschalk S, Savoldo B, Brenner M K. Design and development of therapies using chimeric antigen receptor-expressing T cells. Immunol Rev 2014; 257:107-26.
21. June C H, Sadelain M. Chimeric Antigen Receptor Therapy. The New England journal of medicine 2018; 379:64-73.
22. Ongaro T, Matasci M, Cazzamalli S, et al. A novel anti-cancer L19-interleukin-12 fusion protein with an optimized peptide linker efficiently localizes in vivo at the site of tumors. J Biotechnol 2019; 291:17-25.
23. White E S, Baralle F E, Muro A F. New insights into form and function of fibronectin splice variants. J Pathol 2008; 216:1-14.
24. White E S, Muro A F. Fibronectin splice variants: understanding their multiple roles in health and disease using engineered mouse models. IUBMB Life 2011; 63:538-46.
25. Mariani G, Lasku A, Pau A, et al. A pilot pharmacokinetic and immunoscintigraphic study with the technetium-99m-labeled monoclonal antibody BC-1 directed against oncofetal fibronectin in patients with brain tumors. Cancer 1997; 80:2484-9.
26. Berndorff D, Borkowski S, Moosmayer D, et al. Imaging of tumor angiogenesis using 99mTc-labeled human recombinant anti-ED-B fibronectin antibody fragments. J Nucl Med 2006; 47:1707-16.
27. Borsi L, Balza E, Bestagno M, et al. Selective targeting of tumoral vasculature: comparison of different formats of an antibody (L19) to the ED-B domain of fibronectin. Int J Cancer 2002; 102:75-85.
28. Pini A, Viti F, Santucci A, et al. Design and use of a phage display library. Human antibodies with subnanomolar affinity against a marker of angiogenesis eluted from a two-dimensional gel. J Biol Chem 1998; 273:21769-76.
29. Tijink B M, Perk L R, Budde M, et al. (124) I-L19-SIP for immuno-PET imaging of tumour vasculature and guidance of (131) I-L19-SIP radioimmunotherapy. Eur J Nucl Med Mol Imaging 2009; 36:1235-44.
30. Xie Y J, Dougan M, Jailkhani N, et al. Nanobody-based CAR T cells that target the tumor microenvironment inhibit the growth of solid tumors in immunocompetent mice. Proceedings of the National Academy of Sciences of the United States of America 2019; 116:7624-31.
31. Posthumadeboer J, Piersma S R, Pham TV, et al. Surface proteomic analysis of osteosarcoma identifies EPHA2 as receptor for targeted drug delivery. British journal of cancer 2013; 109:2142-54.
32. Nordberg J, Mpindi J P, Iljin K, et al. Systemic analysis of gene expression profiles identifies ErbB3 as a potential drug target in pediatric alveolar rhabdomyosarcoma. PloS one 2012; 7: e50819.
33. Morsut L, Roybal K T, Xiong X, et al. Engineering Customized Cell Sensing and Response Behaviors Using Synthetic Notch Receptors. Cell 2016; 164:780-91.
34. Roybal K T, Williams J Z, Morsut L, et al. Engineering T Cells with Customized Therapeutic Response Programs Using Synthetic Notch Receptors. Cell 2016; 167:419-32 e16.
35. To W S, Midwood K S. Plasma and cellular fibronectin: distinct and independent functions during tissue repair. Fibrogenesis Tissue Repair 2011; 4:21.
36. Krenciute G, Prinzing B L, Yi Z, et al. Transgenic Expression of IL15 Improves Antiglioma Activity of IL13Ralpha2-CAR T Cells but Results in Antigen Loss Variants. Cancer immunology research 2017; 5:571-81.
37. Krenciute G, Krebs S, Torres D, et al. Characterization and Functional Analysis of scFv-based Chimeric Antigen Receptors to Redirect T Cells to IL13Ralpha2-positive Glioma. Molecular therapy: the journal of the American Society of Gene Therapy 2016; 24:354-63.
38. Yi Z, Prinzing B L, Cao F, Gottschalk S, Krenciute G. Optimizing EphA2-CAR T Cells for the Adoptive Immunotherapy of Glioma. Mol Ther Methods Clin Dev 2018; 9:70-80.
39. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer discovery 2017; 7:1306-19.
40. Koneru M, Purdon T J, Spriggs D, Koneru S, Brentjens R J. IL-12 secreting tumor-targeted chimeric antigen receptor T cells eradicate ovarian tumors in vivo. Oncoimmunology 2015; 4: e994446.
41. Chmielewski M, Abken H. CAR T Cells Releasing IL-18 Convert to T-Bet (high) FoxO1 (low) Effectors that Exhibit Augmented Activity against Advanced Solid Tumors. Cell reports 2017; 21:3205-19.
42. Zhao S, Kurenbekova L, Gao Y, et al. NKD2, a negative regulator of Wnt signaling, suppresses tumor growth and metastasis in osteosarcoma. Oncogene 2015; 34:5069-79.
43. Di Stasi A, Tey S K, Dotti G, et al. Inducible apoptosis as a safety switch for adoptive cell therapy. NEnglJMed 2011; 365:1673-83.
44. Falcon C, Al-Obaidi M, Di Stasi A. Exploiting Cell Death Pathways for Inducible Cell Elimination to Modulate Graft-versus-Host-Disease. Biomedicines 2017; 5.
45. Bonifant C L, Szoor A, Torres D, et al. CD123-Engager T Cells as a Novel Immunotherapeutic for Acute Myeloid Leukemia. Molecular therapy: the journal of the American Society of Gene Therapy 2016; 24:1615-26.
46. Hedeker D, Gibbons R, Waternaux C. Sample size estimation for longitudinal designs with attrition: comparing time-related contrasts between two groups. Journal of Educational and Behavioral Statistics 1999:70-93.
47. Orlando E J, Han X, Tribouley C, et al. Genetic mechanisms of target antigen loss in CAR19 therapy of acute lymphoblastic leukemia. Nature medicine 2018; 24:1504-6.
48. Yoshida T, Akatsuka T, Imanaka-Yoshida K. Tenascin-C and integrins in cancer. Cell Adh Migr 2015; 9:96-104.
49. Spenle C, Saupe F, Midwood K, Burckel H, Noel G, Orend G. Tenascin-C: Exploitation and collateral damage in cancer management. Cell Adh Migr 2015; 9:141-53.
50. Allen-Rhoades W, Whittle 5B, and Rainusso N. Pediatric Solid Tumors in Children and Adolescents: An Overview. Pediatr Rev. 2018; 39 (9): 444-53.
51. Ostrom Q T, Gittleman H, Xu J, Kromer C, Wolinsky Y, Kruchko C, et al. CBTRUS Statistical Report: Primary Brain and Other Central Nervous System Tumors Diagnosed in the United States in 2009-2013. Neuro Oncol. 2016; 18 (suppl_5): v1-v75.
52. Ostrom Q T, Gittleman H, Liao P, Vecchione-Koval T, Wolinsky Y, Kruchko C, et al. CBTRUS Statistical Report: Primary brain and other central nervous system tumors diagnosed in the United States in 2010-2014. Neuro Oncol. 2017; 19 (suppl_5): v1-v88.
53. Mackay A, Burford A, Carvalho D, Izquierdo E, Fazal-Salom J, Taylor K R, et al. Integrated Molecular Meta-Analysis of 1,000 Pediatric High-Grade and Diffuse Intrinsic Pontine Glioma. Cancer Cell. 2017; 32 (4): 520-37.e5.
54. Jones C, Karajannis M A, Jones D T W, Kieran M W, Monje M, Baker S J, et al.

Pediatric high-grade glioma: biologically and clinically in need of new thinking. Neuro Oncol. 2017; 19 (2): 153-61.
55. Sadelain M, Brentjens R, and Rivière I. The basic principles of chimeric antigen receptor design. Cancer Discov. 2013; 3 (4): 388-98.
56. Muhammad N, Mao Q, and Xia H. CAR T-cells for cancer therapy. Biotechnol Genet Eng Rev. 2017; 33 (2): 190-226.
57. Brocker T, and Karjalainen K. Signals through T cell receptor-zeta chain alone are insufficient to prime resting T lymphocytes. J Exp Med. 1995; 181 (5): 1653-9.
58. Segal N H, Parsons D W, Peggs K S, Velculescu V, Kinzler K W, Vogelstein B, et al. Epitope landscape in breast and colorectal cancer. Cancer Res. 2008; 68 (3): 889-92.
59. Brudno J N, and Kochenderfer J N. Toxicities of chimeric antigen receptor T cells: recognition and management. Blood. 2016; 127 (26): 3321-30.
60. J. P. Connelly, S. M. Pruett-Miller, CRIS.py: A Versatile and High-throughput Analysis Program for CRISPR-based Genome Editing. Sci Rep 9, 4194 (2019).
61. S. Kakarla et al., Antitumor effects of chimeric receptor engineered human T cells directed to tumor stroma. Mol Ther 21, 1611-1620 (2013).
62. S. Gottschalk et al., Generating CTLs against the subdominant Epstein-Barr virus LMP1 antigen for the adoptive immunotherapy of EBV-associated malignancies. Blood 101, 1905-1912 (2003).
63. M. Midulla et al., Source of oncofetal ED-β-containing fibronectin: implications of production by both tumor and endothelial cells. Cancer Res 60, 164-169 (2000).
64. S. A. Grupp et al., Chimeric antigen receptor-modified T cells for acute lymphoid leukemia. N Engl J Med 368, 1509-1518 (2013).
65. T. J. Fry et al., CD22-targeted CAR T cells induce remission in β-ALL that is naive or resistant to CD19-targeted CAR immunotherapy. Nat Med 24, 20-28 (2018).
66. C. A. Ramos et al., Clinical and immunological responses after CD30-specific chimeric antigen receptor-redirected lymphocytes. J Clin Invest 127, 3462-3471 (2017).
67. M. Martinez, E. K. Moon, CAR T Cells for Solid Tumors: New Strategies for Finding, Infiltrating, and Surviving in the Tumor Microenvironment. Front Immunol 10, 128 (2019).
68. D. Hanahan, R. A. Weinberg, Hallmarks of cancer: the next generation. Cell 144, 646-674 (2011).
69. M. Kaspar, L. Zardi, D. Neri, Fibronectin as target for tumor therapy. Int J Cancer 118, 1331-1339 (2006).
70. Z. A. Khan et al., EDB fibronectin and angiogenesis—a novel mechanistic pathway. Angiogenesis 8, 183-196 (2005).
71. E. El-Emir et al., Characterisation and radioimmunotherapy of L19-SIP, an anti-angiogenic antibody against the extra domain B of fibronectin, in colorectal tumour models. Br J Cancer 96, 1862-1870 (2007).
72. D. Moosmayer et al., Bispecific antibody pretargeting of tumor neovasculature for improved systemic radiotherapy of solid tumors. Clin Cancer Res 12, 5587-5595 (2006).
73. S. P. Santoro et al., T cells bearing a chimeric antigen receptor against prostate-specific membrane antigen mediate vascular disruption and result in tumor regression. Cancer Immunol Res 3, 68-84 (2015).
74. W. Wang et al., Specificity redirection by CAR with human VEGFR-1 affinity endows T lymphocytes with tumor-killing ability and anti-angiogenic potency. Gene Ther 20, 970978 (2013).
75. K. Petrovic et al., TEM8/ANTXR1-specific CAR T cells mediate toxicity in vivo. PLOS One 14, e0224015 (2019).
76. J. P. Venables, Aberrant and alternative splicing in cancer. Cancer Res 64, 7647-7654 (2004).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-51. (canceled)

52. A chimeric antigen receptor (CAR) comprising,
(a) an extracellular target-binding domain comprising a Fibronectin Extradomain B (FN-EDB)-binding moiety,
(b) a hinge domain,
(c) a transmembrane domain, and
(d) a cytoplasmic domain comprising
(i) optionally one or more costimulatory domains, and
(ii) a lymphocyte activation domain.

53. An isolated host cell, comprising a polynucleotide sequence encoding a chimeric antigen receptor (CAR) targeting a Fibronectin Extradomain B (FN-EDB) and at least one additional polynucleotide sequence encoding a second gene, wherein the CAR comprises:
(a) an extracellular target-binding domain comprising a Fibronectin Extradomain B (FN-EDB)-binding moiety,
(b) a hinge domain,
(c) a transmembrane domain, and
(d) a cytoplasmic domain comprising
(i) optionally one or more costimulatory domains, and
(ii) a lymphocyte activation domain.

54. An isolated host cell, comprising a chimeric antigen receptor (CAR) comprising the amino acid sequence of SEQ ID NO: 95, or an amino acid sequence having at least 80% identity thereof.

55. The isolated host cell of claim 53, wherein the host cell is an immune cell.

56. The isolated host cell of claim 55, wherein the immune cell is a T-cell, a NK cell, or a macrophage.

57. (canceled)

58. The isolated host cell of claim 53, wherein the host cell further comprises a second CAR or a bispecific molecule and wherein the expression of the second CAR or the bispecific molecule is induced by the activation of the first CAR.

59. The isolated host cell of claim 58, wherein the expression of the second CAR or bispecific molecule is induced using a synthetic Notch (synNotch) receptor.

60. The isolated host cell of claim 58, wherein the expression of the second CAR or bispecific molecule is induced using at least one nuclear factor of activated T-cells (NFAT) dependent-promoter.

61. The isolated host cell of claim 58, wherein the bispecific molecule is a bispecific T-cell engager (BiTE) or a bispecific antibody.

62. The isolated host cell of claim 58, wherein the second CAR or the bispecific molecule targets a tumor associated antigen.

63. (canceled)

64. (canceled)

65. The isolated host cell of any one of claim 53, wherein the host cell is an allogeneic cell or autologous.

66.-70. (canceled)

71. A pharmaceutical composition comprising the host cell of claim 53 and a pharmaceutically acceptable carrier and/or excipient.

72.-76. (canceled)

77. A method for indirectly killing a tumor cell expressing a tumor associated antigen, said method comprising contacting said cell with the pharmaceutical composition of claim 71, wherein the host cell(s) further comprises a second CAR or a bispecific molecule, wherein the expression of the second CAR or the bispecific molecule is induced by the activation of the first CAR, and wherein the second CAR or the bispecific molecule targets the tumor associated antigen.

78. A method for treating a tumor in a subject in need thereof, wherein one or more cells of the tumor express FN-EDB, said method comprising administering to the subject a therapeutically effective amount of the pharmaceutical composition of claim 71.

79. A method for treating a tumor and/or inhibiting tumor neovascularization in a subject in need thereof, wherein one or more cells of the tumor or non-tumor cells within the tumor micro-environment express FN-EDB and the tumor cell expresses a tumor associated antigen, said method comprising administering to the subject a therapeutically effective amount of the pharmaceutical composition of claim 71.

80.-86. (canceled)

87. The isolated host cell of claim 53, wherein the second gene encodes a truncated CD19 (tCD19) polypeptide.

88. The isolated host cell of claim 53, wherein the second gene encodes a synthetic notch (synNotch) receptor.

89. The isolated host cell of claim 88, wherein the synNotch receptor is operably linked to a polynucleotide that encodes an antigen binding moiety.

90. The isolated host cell of claim 53, wherein the FN-EDB-binding moiety is an anti-FN-EDB single chain variable fragment (scFv).

91. The isolated host cell of claim 90, wherein the anti-FN-EDB scFv is derived from antibody L19 (L19 scFv).


SEQUENCE LISTING (129 sequences)

SEQ ID NO: 1 - protein, 807 aa, synthetic construct
  REGION 1..807: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 2 - DNA, 2421 bp, synthetic construct
  misc_feature 1..2421: note = Description of Artificial Sequence: Synthetic polynucleotide
  source 1..2421: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 3 - protein, 485 aa, synthetic construct
  REGION 1..485: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1455 bp, synthetic construct
  misc_feature 1..1455: note = Description of Artificial Sequence: Synthetic polynucleotide
  source 1..1455: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaaccgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 5 - protein, 486 aa, synthetic construct
  REGION 1..486: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1458 bp, synthetic construct
  misc_feature 1..1458: note = Description of Artificial Sequence: Synthetic polynucleotide
  source 1..1458: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaacaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 7 - protein, 480 aa, synthetic construct
  REGION 1..480: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 1440 bp, synthetic construct
  misc_feature 1..1440: note = Description of Artificial Sequence: Synthetic polynucleotide
  source 1..1440: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaaccgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 9 - protein, 680 aa, synthetic construct
  REGION 1..680: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 2037 bp, synthetic construct
  misc_feature 1..2037: note = Description of Artificial Sequence: Synthetic polynucleotide
  source 1..2037: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaacgctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 11 - protein, 456 aa, synthetic construct
  REGION 1..456: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1368 bp, synthetic construct
  misc_feature 1..1368: note = Description of Artificial Sequence: Synthetic polynucleotide
  source 1..1368: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 13 - protein, 450 aa, synthetic construct
  REGION 1..450: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 1350 bp, synthetic construct
  misc_feature 1..1350: note = Description of Artificial Sequence: Synthetic polynucleotide
  source 1..1350: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 15 - protein, 649 aa, synthetic construct
  REGION 1..649: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 1947 bp, synthetic construct
  misc_feature 1..1947: note = Description of Artificial Sequence: Synthetic polynucleotide
  source 1..1947: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 17 - protein, 474 aa, synthetic construct
  REGION 1..474: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - DNA, 1421 bp, synthetic construct
  misc_feature 1..1421: note = Description of Artificial Sequence: Synthetic polynucleotide
  source 1..1421: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 19 - protein, 475 aa, synthetic construct
  REGION 1..475: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 1424 bp, synthetic construct
  misc_feature 1..1424: note = Description of Artificial Sequence: Synthetic polynucleotide
  source 1..1424: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 21 - protein, 469 aa, synthetic construct
  REGION 1..469: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1406 bp, synthetic construct
  misc_feature 1..1406: note = Description of Artificial Sequence: Synthetic polynucleotide
  source 1..1406: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgcgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 23 - protein, 668 aa, synthetic construct
  REGION 1..668: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 2003 bp, synthetic construct
  misc_feature 1..2003: note = Description of Artificial Sequence: Synthetic polynucleotide
  source 1..2003: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 25 - protein, 236 aa, synthetic construct
  REGION 1..236: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 26 - DNA, 708 bp, synthetic construct
  misc_feature 1..708: note = Description of Artificial Sequence: Synthetic polynucleotide
  source 1..708: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaag

SEQ ID NO: 27 - protein, 15 aa, synthetic construct
  REGION 1..15: note = Description of Artificial Sequence: Synthetic peptide
EPKSCDKTHTCPPCP

SEQ ID NO: 28 - DNA, 45 bp, synthetic construct
  misc_feature 1..45: note = Description of Artificial Sequence: Synthetic oligonucleotide
  source 1..45: mol_type = other DNA
gagcccaaatcttgtgacaaaactcacacatgcccaccgtgcccg

SEQ ID NO: 29 - protein, 12 aa, synthetic construct
  REGION 1..12: note = Description of Artificial Sequence: Synthetic peptide
ERKCCVECPPCP

SEQ ID NO: 30 - protein, 62 aa, synthetic construct
  REGION 1..62: note = Description of Artificial Sequence: Synthetic polypeptide
ELKTPLGDTTHTCPRCPEPKSCDTPPPCPRCPEPKSCDTPPPCPRCPEPKSCDTPPPCPRCP

SEQ ID NO: 31 - protein, 12 aa, synthetic construct
  REGION 1..12: note = Description of Artificial Sequence: Synthetic peptide
ESKYGPPCPSCP

SEQ ID NO: 32 - protein, 47 aa, synthetic construct
  REGION 1..47: note = Description of Artificial Sequence: Synthetic polypeptide
PAKPTTTPAPRPPTPAPTIASQPLSLRPEACRPAAGGAVHTRGLDFA

SEQ ID NO: 33 - DNA, 231 bp, synthetic construct
  misc_feature 1..231: note = Description of Artificial Sequence: Synthetic polynucleotide
  source 1..231: mol_type = other DNA
ccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaac

SEQ ID NO: 34 - protein, 39 aa, synthetic construct
  REGION 1..39: note = Description of Artificial Sequence: Synthetic polypeptide
IEVMYPPPYLDNEKSNGTIIHVKGKHLCPSPLFPGPSKP

SEQ ID NO: 35 - DNA, 116 bp, synthetic construct
  misc_feature 1..116: note = Description of Artificial Sequence: Synthetic polynucleotide
  source 1..116: mol_type = other DNA
atcgaagtgatgtacccgcctccttacctggacaacgagaagtccaacggcaccatcatccacgtgaagggaaagcacctgtgtccttctccactgttccccggacctagcaagcc

SEQ ID NO: 36 - protein, 27 aa, synthetic construct
  REGION 1..27: note = Description of Artificial Sequence: Synthetic peptide
FWVLVVVGGVLACYSLLVTVAFIIFWV

SEQ ID NO: 37 - DNA, 81 bp, synthetic construct
  misc_feature 1..81: note = Description of Artificial Sequence: Synthetic oligonucleotide
  source 1..81: mol_type = other DNA
ttctgggtgctggtggtcgtgggcggagtgctggcctgttacagcctgctcgtgaccgtggccttcatcatcttttgggtg

SEQ ID NO: 38 - protein, 30 aa, synthetic construct
  REGION 1..30: note = Description of Artificial Sequence: Synthetic polypeptide
CDIYIWAPLAGTCGVLLLSLVITLYCNHRN

SEQ ID NO: 39 - DNA, 90 bp, synthetic construct
  misc_feature 1..90: note = Description of Artificial Sequence: Synthetic oligonucleotide
  source 1..90: mol_type = other DNA
tgcgacatctacatctgggcccctctggccggcacatgtggcgtgctgctgctgagcctcgtgatcaccctgtactgcaaccaccggaac

SEQ ID NO: 40 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 41 - DNA, 125 bp, synthetic construct
  misc_feature 1..125: note = Description of Artificial Sequence: Synthetic polynucleotide
  source 1..125: mol_type = other DNA
cgcagcaagcggagccggctgctgcacagcgactacatgaacatgacccccagacggcctggccccaccagaaagcactaccagccttacgcccctcccagagacttcgccgcctaccggtccag

SEQ ID NO: 42 - DNA, 123 bp, synthetic construct
  misc_feature 1..123: note = Description of Artificial Sequence: Synthetic polynucleotide
  source 1..123: mol_type = other DNA
cgaagcaagcggagccggctgctgcacagcgactacatgaacatgacccctagacggcccggaccaaccagaaagcactaccagccttacgctcctcctagagatttcgccgcctaccggtcc

SEQ ID NO: 43 - DNA, 123 bp, synthetic construct
  misc_feature 1..123: note = Description of Artificial Sequence: Synthetic polynucleotide
  source 1..123: mol_type = other DNA
cggtccaagagaagcagactgctgcacagcgactacatgaacatgacccctagacggcccggacctaccagaaagcactaccagccttacgctcctcctagagatttcgccgcctaccggtcc

SEQ ID NO: 44 - DNA, 123 bp, synthetic construct
  misc_feature 1..123: note = Description of Artificial Sequence: Synthetic polynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 45 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 46 - DNA, 126 bp, synthetic construct
  misc_feature 1..126: note = Description of Artificial Sequence: Synthetic polynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 47 - protein, 36 aa, synthetic construct
  REGION 1..36: note = Description of Artificial Sequence: Synthetic polypeptide
RDQRLPPDAHKPPGGGSFRTPIQEEQADAHSTLAKI

SEQ ID NO: 48 - DNA, 108 bp, synthetic construct
  misc_feature 1..108: note = Description of Artificial Sequence: Synthetic polynucleotide
  source 1..108: mol_type = other DNA
agggaccagaggctgccccccgatgcccacaagccccctgggggaggcagtttccggacccccatccaagaggagcaggccgacgcccactccaccctggccaagatc

SEQ ID NO: 49 - protein, 171 aa, synthetic construct
  REGION 1..171: note = Description of Artificial Sequence: Synthetic polypeptide
AAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 50 - DNA, 513 bp, synthetic construct
  misc_feature 1..513: note = Description of Artificial Sequence: Synthetic polynucleotide
  source 1..513: mol_type = other DNA
gctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatc

SEQ ID NO: 51 - protein, 172 aa, synthetic construct
  REGION 1..172: note = Description of Artificial Sequence: Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 52 - DNA, 516 bp, synthetic construct
  misc_feature 1..516: note = Description of Artificial Sequence: Synthetic polynucleotide
  source 1..516: mol_type = other DNA
atggccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 53 - protein, 171 aa, synthetic construct
  REGION 1..171: note = Description of Artificial Sequence: Synthetic polypeptide
AAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 54 - DNA, 513 bp, synthetic construct
  misc_feature 1..513: note = Description of Artificial Sequence: Synthetic polynucleotide
  source 1..513: mol_type = other DNA
gccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 55 - protein, 62 aa, synthetic construct
  REGION 1..62: note = Description of Artificial Sequence: Synthetic polypeptide
KKVAKKPTNKAPHPKQEPQEINFPDDLPGSNTAAPVQETLHGCQPVTQEDGKESRISVQERQ

SEQ ID NO: 56 - DNA, 186 bp, synthetic construct
  misc_feature 1..186: note = Description of Artificial Sequence: Synthetic polynucleotide
  source 1..186: mol_type = other DNA
aagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccag

SEQ ID NO: 57 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 336 bp, synthetic construct
  misc_feature 1..336: note = Description of Artificial Sequence: Synthetic polynucleotide
  source 1..336: mol_type = other DNA
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 59 - protein, 117 aa, synthetic construct
  REGION 1..117: note = Description of Artificial Sequence: Synthetic polypeptide
RVKFSRSADAPAYQQGQNQLYNELNLGRREEYDVLDKRRGRDRRGRDPEMGGKPRRKNPQEGLYNELQKDKMAEAYSEIGMKGERRRGKGHDGLYQGLSTATKDTYDALHMQALPPR

SEQ ID NO: 60 - DNA, 351 bp, synthetic construct
  misc_feature 1..351: note = Description of Artificial Sequence: Synthetic polynucleotide
  source 1..351: mol_type = other DNA
agagtgaagttcagcaggagcgcagacgcccccgcgtaccagcagggccagaaccagctctataacgagctcaatctaggacgaagagaggagtacgatgttttggacaagagacgtggccgggacagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 61 - protein, 19 aa, synthetic construct
  REGION 1..19: note = Description of Artificial Sequence: Synthetic peptide
MDWIWRILFLVGAATGAHS

SEQ ID NO: 62 - DNA, 57 bp, synthetic construct
  misc_feature 1..57: note = Description of Artificial Sequence: Synthetic oligonucleotide
  source 1..57: mol_type = other DNA
atggactggatctggcgcatcctcttcctcgtcggcgctgctaccggcgctcattct

SEQ ID NO: 63 - protein, 21 aa, synthetic construct
  REGION 1..21: note = Description of Artificial Sequence: Synthetic peptide
MALPVTALLLPLALLLHAARP

SEQ ID NO: 64 - DNA, 63 bp, synthetic construct
  misc_feature 1..63: note = Description of Artificial Sequence: Synthetic oligonucleotide
  source 1..63: mol_type = other DNA
atggccttaccagtgaccgccttgctcctgccgctggccttgctgctccacgccgccaggccg

SEQ ID NO: 65 - protein, 333 aa, synthetic construct
  REGION 1..333: note = Description of Artificial Sequence: Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 66 - DNA, 999 bp, synthetic construct
  misc_feature 1..999: note = Description of Artificial Sequence: Synthetic polynucleotide
  source 1..999: mol_type = other DNA
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 67 - protein, 336 aa, synthetic construct
  REGION 1..336: note = Description of Artificial Sequence: Synthetic polypeptide
ILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRR

SEQ ID NO: 68 - DNA, 1008 bp, synthetic construct
  misc_feature 1..1008: note = Description of Artificial Sequence: Synthetic polynucleotide
  source 1..1008: mol_type = other DNA
attctggattatagctttggcggcggcgcgggccgcgatattccgccgccgctgattgaagaagcgtgcgaactgccggaatgccaggaagatgcgggcaacaaagtgtgcagcctgcagtgcaacaaccatgcgtgcggctgggatggcggcgattgcagcctgaactttaacgatccgtggaaaaactgcacccagagcctgcagtgctggaaatattttagcgatggccattgcgatagccagtgcaacagcgcgggctgcctgtttgatggctttgattgccagcgcgcggaaggccagtgcaacccgctgtatgatcagtattgcaaagatcattttagcgatggccattgcgatcagggctgcaacagcgcggaatgcgaatgggatggcctggattgcgcggaacatgtgccggaacgcctggcggcgggcaccctggtggtggtggtgctgatgccgccggaacagctgcgcaacagcagctttcattttctgcgcgaactgagccgcgtgctgcataccaacgtggtgtttaaacgcgatgcgcatggccagcagatgatttttccgtattatggccgcgaagaagaactgcgcaaacatccgattaaacgcgcggcggaaggctgggcggcgccggatgcgctgctgggccaggtgaaagcgagcctgctgccgggcggcagcgaaggcggccgccgccgccgcgaactggatccgatggatgtgcgcggcagcattgtgtatctggaaattgataaccgccagtgcgtgcaggcgagcagccagtgctttcagagcgcgaccgatgtggcggcgtttctgggcgcgctggcgagcctgggcagcctgaacattccgtataaaattgaagcggtgcagagcgaaaccgtggaaccgccgccgccggcgcagctgcattttatgtatgtggcggcggcggcgtttgtgctgctgttttttgtgggctgcggcgtgctgctgagccgcaaacgccgccgc

SEQ ID NO: 69 - protein, 19 aa, Thosea asigna virus
AEGRGSLLTCGDVEENPGP

SEQ ID NO: 70 - DNA, 57 bp, Thosea asigna virus
  source 1..57: mol_type = unassigned DNA
gccgagggcagaggcagcctgctgacatgtggcgacgtggaagagaacccaggcccc

SEQ ID NO: 71 - protein, 18 aa, Thosea asigna virus
EGRGSLLTCGDVEENPGP

SEQ ID NO: 72 - DNA, 54 bp, Thosea asigna virus
  source 1..54: mol_type = unassigned DNA
gaaggcagaggatcactgctgacatgcggcgacgtggaagagaaccctggaccc

SEQ ID NO: 73 - protein, 21 aa, synthetic construct
  REGION 1..21: note = Description of Artificial Sequence: Synthetic peptide
GSGEGRGSLLTCGDVEENPGP

SEQ ID NO: 74 - protein, 63 aa, synthetic construct
  REGION 1..63: note = Description of Artificial Sequence: Synthetic polypeptide
GSGSRVTELLYRMKRAETYCPRPLLAIHPTEARHKQKIVAPVKQLLNFDLLKLAGDVESNPGP

SEQ ID NO: 75 - protein, 19 aa, Amphimedon sp.
  source 1..19: note = Amphimedon queenslandica
LLCFLLLLLSGDVELNPGP

SEQ ID NO: 76 - protein, 20 aa, Amphimedon sp.
  source 1..20: note = Amphimedon queenslandica
HHFMFLLLLLAGDIELNPGP

SEQ ID NO: 77 - protein, 20 aa, Saccoglossus kowalevskii
WFLVLLSFILSGDIEVNPGP

SEQ ID NO: 78 - protein, 20 aa, Branchiostoma floridae
KNCAMYMLLLSGDVETNPGP

SEQ ID NO: 79 - protein, 20 aa, Branchiostoma floridae
MVISQLMLKLAGDVEENPGP

SEQ ID NO: 80 - protein, 22 aa, synthetic construct
  REGION 1..22: note = Description of Artificial Sequence: Synthetic peptide
GSGATNFSLLKQAGDVEENPGP

SEQ ID NO: 81 - protein, 23 aa, synthetic construct
  REGION 1..23: note = Description of Artificial Sequence: Synthetic peptide
GSGQCTNYALLKLAGDVESNPGP

SEQ ID NO: 82 - protein, 8 aa, unidentified
  REGION 1..8: note = Description of Unknown: 2A consensus sequence
  VARIANT 2: note = Any amino acid
  VARIANT 4: note = Any amino acid
DXEXNPGP

SEQ ID NO: 83 - DNA, 54 bp, Thosea asigna virus
  source 1..54: mol_type = unassigned DNA
gagggcagaggcagcctgctgacatgtggcgacgtggaagagaacccaggcccc

SEQ ID NO: 84 - protein, 297 aa, synthetic construct
  REGION 1..297: note = Description of Artificial Sequence: Synthetic polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 85 - DNA, 891 bp, synthetic construct
  misc_feature 1..891: note = Description of Artificial Sequence: Synthetic polynucleotide
  source 1..891: mol_type = other DNA
atgaccacacctcggaacagcgtgaacggcacatttcccgccgagcctatgaagggccctatcgccatgcagtctggccccaagcctctgttcagacggatgtctagcctcgtgggccccacacagagctttttcatgagagagagcaagaccctgggcgccgtgcagatcatgaacggcctgtttcacattgccctcggcggcctgctgatgatccctgccggaatctatgcccctatctgcgtgaccgtgtggtatcctctgtggggcggcatcatgtacatcatctctggatctctgctggccgccaccgagaagaacagcagaaagtgtctggtcaagggcaagatgatcatgaatagcctgagcctgttcgccgccatcagcggcatgatcctgagcatcatggatatcctgaatatcaagatcagccacttcctgaagatggaaagcctgaacttcatcagggcccacacaccttacatcaacatctacaactgcgagcccgccaatcctagcgagaagaatagccccagcacacagtactgctactctatccagagcctgtttctgggcatcctgagcgtgatgctgatcttcgcattcttccaagagctggttatcgccggcatcgtggaaaacgagtggaagcggacctgcagcagacccaagagcaacatcgtgctgctgagcgccgaggaaaagaaagagcagaccatcgagatcaaagaggaagtcgtcggcctgaccgagacaagcagccagcctaagaacgaagaggacattgagatcatccccatccaagaagaggaagaagaagagactgagacaaacttccccgagcctcctcaggaccaagagagcagccccattgagaacgatagcagccct

SEQ ID NO: 86 - DNA, 894 bp, synthetic construct
  misc_feature 1..894: note = Description of Artificial Sequence: Synthetic polynucleotide
  source 1..894: mol_type = other DNA
atgaccacccccagaaacagcgtgaacggcaccttccccgccgagcctatgaagggccctatcgccatgcagagcggccccaagcccctgttcagacggatgtctagcctcgtgggccctacccagagcttcttcatgagagagagcaagaccctgggcgccgtgcagatcatgaacggcctgttccacattgccctgggcggcctgctgatgatccctgccggaatctacgcccccatctgcgtgaccgtgtggtatcctctgtggggcggcatcatgtacatcatcagcggcagcctgctggccgccaccgagaagaacagcagaaagtgcctcgtgaagggcaagatgatcatgaatagcctgagcctgttcgccgccatctccggcatgatcctgagcatcatggatatcctgaatatcaagatcagccacttcctgaagatggaaagcctgaacttcatccgggcccacaccccctacatcaacatctacaactgcgagcccgccaaccccagcgagaagaatagccccagcacccagtactgctactctatccagtccctgttcctgggcatcctgagcgtgatgctgatcttcgcattttttcaagagctcgtgatcgccggcatcgtggaaaacgagtggaagcggacctgcagccggcccaagagcaacatcgtgctgctgagcgccgaggaaaagaaagagcagaccatcgagatcaaagaggaagtcgtgggcctgaccgagacaagctcccagcccaagaacgaagaggacattgagatcatcccaatccaggaagaagaggaagaggaaaccgagactaacttccccgagcccccccaggaccaggaaagcagccccatcgagaacgacagcagcccctga

SEQ ID NO: 87 - protein, 159 aa, synthetic construct
  REGION 1..159: note = Description of Artificial Sequence: Synthetic polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGS

SEQ ID NO: 88 - DNA, 477 bp, synthetic construct
  misc_feature 1..477: note = Description of Artificial Sequence: Synthetic polynucleotide
  source 1..477: mol_type = other DNA
atgaaactgctgagcagcattgaacaggcgtgcgatatttgccgcctgaaaaaactgaaatgcagcaaagaaaaaccgaaatgcgcgaaatgcctgaaaaacaactgggaatgccgctatagcccgaaaaccaaacgcagcccgctgacccgcgcgcatctgaccgaagtggaaagccgcctggaacgcctggaacagctgtttctgctgatttttccgcgcgaagatctggatatgattctgaaaatggatagcctgcaggatattaaagcgctgctgaccggcctgtttgtgcaggataacgtgaacaaagatgcggtgaccgatcgcctggcgagcgtggaaaccgatatgccgctgaccctgcgccagcatcgcattagcgcgaccagcagcagcgaagaaagcagcaacaaaggccagcgccagctgaccgtgagcgcggcggcgggcggcagcggcggcagcggcggcagc

SEQ ID NO: 89 - protein, 50 aa, synthetic construct
  REGION 1..50: note = Description of Artificial Sequence: Synthetic polypeptide
DALDDFDLDMLGSDALDDFDLDMLGSDALDDFDLDMLGSDALDDFDLDML

SEQ ID NO: 90 - DNA, 150 bp, synthetic construct
  misc_feature 1..150: note = Description of Artificial Sequence: Synthetic polynucleotide
  source 1..150: mol_type = other DNA
gatgcgctggatgattttgatctggatatgctgggcagcgatgcgctggatgattttgatctggatatgctgggcagcgatgcgctggatgattttgatctggatatgctgggcagcgatgcgctggatgattttgatctggatatgctg

SEQ ID NO: 91 - protein, 20 aa, synthetic construct
  REGION 1..20: note = Description of Artificial Sequence: Synthetic peptide
DLEPKSCDKTHTCPPCPDPK

SEQ ID NO: 92 - DNA, 60 bp, synthetic construct
  misc_feature 1..60: note = Description of Artificial Sequence: Synthetic oligonucleotide
  source 1..60: mol_type = other DNA
gatctcgagcccaaatcttgtgacaaaactcacacatgcccaccgtgcccggatcccaag

SEQ ID NO: 93 - protein, 800 aa, synthetic construct
  REGION 1..800: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDML

SEQ ID NO: 94 - DNA, 2400 bp, synthetic construct
  misc_feature 1..2400: note = Description of Artificial Sequence: Synthetic polynucleotide
  source 1..2400: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagattctggattatagctttggcggcggcgcgggccgcgatattccgccgccgctgattgaagaagcgtgcgaactgccggaatgccaggaagatgcgggcaacaaagtgtgcagcctgcagtgcaacaaccatgcgtgcggctgggatggcggcgattgcagcctgaactttaacgatccgtggaaaaactgcacccagagcctgcagtgctggaaatattttagcgatggccattgcgatagccagtgcaacagcgcgggctgcctgtttgatggctttgattgccagcgcgcggaaggccagtgcaacccgctgtatgatcagtattgcaaagatcattttagcgatggccattgcgatcagggctgcaacagcgcggaatgcgaatgggatggcctggattgcgcggaacatgtgccggaacgcctggcggcgggcaccctggtggtggtggtgctgatgccgccggaacagctgcgcaacagcagctttcattttctgcgcgaactgagccgcgtgctgcataccaacgtggtgtttaaacgcgatgcgcatggccagcagatgatttttccgtattatggccgcgaagaagaactgcgcaaacatccgattaaacgcgcggcggaaggctgggcggcgccggatgcgctgctgggccaggtgaaagcgagcctgctgccgggcggcagcgaaggcggccgccgccgccgcgaactggatccgatggatgtgcgcggcagcattgtgtatctggaaattgataaccgccagtgcgtgcaggcgagcagccagtgctttcagagcgcgaccgatgtggcggcgtttctgggcgcgctggcgagcctgggcagcctgaacattccgtataaaattgaagcggtgcagagcgaaaccgtggaaccgccgccgccggcgcagctgcattttatgtatgtggcggcggcggcgtttgtgctgctgttttttgtgggctgcggcgtgctgctgagccgcaaacgccgccgcatgaaactgctgagcagcattgaacaggcgtgcgatatttgccgcctgaaaaaactgaaatgcagcaaagaaaaaccgaaatgcgcgaaatgcctgaaaaacaactgggaatgccgctatagcccgaaaaccaaacgcagcccgctgacccgcgcgcatctgaccgaagtggaaagccgcctggaacgcctggaacagctgtttctgctgatttttccgcgcgaagatctggatatgattctgaaaatggatagcctgcaggatattaaagcgctgctgaccggcctgtttgtgcaggataacgtgaacaaagatgcggtgaccgatcgcctggcgagcgtggaaaccgatatgccgctgaccctgcgccagcatcgcattagcgcgaccagcagcagcgaagaaagcagcaacaaaggccagcgccagctgaccgtgagcgcggcggcgggcggcagcggcggcagcggcggcagcgatgcgctggatgattttgatctggatatgctgggcagcgatgcgctggatgattttgatctggatatgctgggcagcgatgcgctggatgattttgatctggatatgctgggcagcgatgcgctggatgattttgatctggatatgctg

SEQ ID NO: 95 - protein, 436 aa, synthetic construct
  REGION 1..436: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 1308 bp, synthetic construct
  misc_feature 1..1308: note = Description of Artificial Sequence: Synthetic polynucleotide
  source 1..1308: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 97 - protein, 466 aa, synthetic construct
  REGION 1..466: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - DNA, 1398 bp, synthetic construct
  misc_feature 1..1398: note = Description of Artificial Sequence: Synthetic polynucleotide
  source 1..1398: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaaccgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 99 - protein, 467 aa, synthetic construct
  REGION 1..467: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1401 bp, synthetic construct
  misc_feature 1..1401: note = Description of Artificial Sequence: Synthetic polynucleotide
  source 1..1401: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaacaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 101 - protein, 461 aa, synthetic construct
  REGION 1..461: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1383 bp, synthetic construct
  misc_feature 1..1383: note = Description of Artificial Sequence: Synthetic polynucleotide
  source 1..1383: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaaccgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 103 - protein, 661 aa, synthetic construct
  REGION 1..661: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKPAKPTTTPAPRPPTPAPTIASQPLSLRPEACRPAAGGAVHTRGLDFACDIYIWAPLAGTCGVLLLSLVITLYCNHRNVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 1980 bp, synthetic construct
  misc_feature 1..1980: note = Description of Artificial Sequence: Synthetic polynucleotide
  source 1..1980: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagccagccaagcccaccacaacccctgctcctagacctcctaccccagcccctaccattgcctcccagccactgtctctgaggcccgaggcttgtagacctgctgcaggcggagccgtgcacaccagaggactggatttcgcctgcgacatctatatctgggcccctctggccggcacctgtggcgtgctgctgctgtcactcgtgatcaccctgtactgcaaccaccggaacgctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 105 - protein, 437 aa, synthetic construct
  REGION 1..437: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 1311 bp, synthetic construct
  misc_feature 1..1311: note = Description of Artificial Sequence: Synthetic polynucleotide
  source 1..1311: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 107 - protein, 431 aa, synthetic construct
  REGION 1..431: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1293 bp, synthetic construct
  misc_feature 1..1293: note = Description of Artificial Sequence: Synthetic polynucleotide
  source 1..1293: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 109 - protein, 630 aa, synthetic construct
  REGION 1..630: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - DNA, 1890 bp, synthetic construct
  misc_feature 1..1890: note = Description of Artificial Sequence: Synthetic polynucleotide
  source 1..1890: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 111 - protein, 455 aa, synthetic construct
  REGION 1..455: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 1364 bp, synthetic construct
  misc_feature 1..1364: note = Description of Artificial Sequence: Synthetic polynucleotide
  source 1..1364: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 113 - protein, 456 aa, synthetic construct
  REGION 1..456: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - DNA, 1367 bp, synthetic construct
  misc_feature 1..1367: note = Description of Artificial Sequence: Synthetic polynucleotide
  source 1..1367: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 115 - protein, 450 aa, synthetic construct
  REGION 1..450: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1349 bp, synthetic construct
  misc_feature 1..1349: note = Description of Artificial Sequence: Synthetic polynucleotide
  source 1..1349: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtgcgcgatcagcgcctgccgccggatgcgcataaaccgccgggcggcggcagctttcgcaccccgattcaggaagaacaggcggatgcgcatagcaccctggcgaaaattcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 117 - protein, 649 aa, synthetic construct
  REGION 1..649: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKIEVMYPPPYLDNEKSNGTIIHVKGKHLCPSPLFPGPSKPFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 1946 bp, synthetic construct
  misc_feature 1..1946: note = Description of Artificial Sequence: Synthetic polynucleotide
  source 1..1946: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagatcgaagtgatgtacccgcctccttacctggacaacgagaagtccaacggcaccatcatccacgtgaagggaaagcacctgtgtccttctccactgttccccggacctagcaagccttctgggtgctggtggtcgtgggcggagtgctggcctgttacagcctgctcgtgaccgtggccttcatcatcttttgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgtggaaaagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccagagagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 119 - protein, 455 aa, synthetic construct
  REGION 1..455: note = Description of Artificial Sequence: Synthetic polypeptide
MDWIWRILFLVGAATGAHSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - DNA, 1365 bp, synthetic construct
  misc_feature 1..1365: note = Description of Artificial Sequence: Synthetic polynucleotide
  source 1..1365: mol_type = other DNA
atggactggatctggcgcatcctcttcctcgtcggcgctgctaccggcgctcattctgaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 121 - protein, 781 aa, synthetic construct
  REGION 1..781: note = Description of Artificial Sequence: Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDML

SEQ ID NO: 122 - DNA, 2343 bp, synthetic construct
  misc_feature 1..2343: note = Description of Artificial Sequence: Synthetic polynucleotide
  source 1..2343: mol_type = other DNA
gaggttcagcttcttgagtctgggggcggcctggtgcaacctggtggcagtcttaggctgagttgcgccgcgtcagggtttactttttcttccttcagcatgtcatgggtccgccaggctccaggtaagggcctggaatgggttagtagcatcagtggcagtagtggtacaacatactacgctgatagtgttaaagggagattcactataagtagggataattctaaaaataccctttatctgcaaatgaattctttgagggccgaagatactgcggtctattactgtgccaaaccttttccatactttgattactggggccaggggacgcttgtcactgtctcctctggcgacggctcctccggcggaagtggaggcgcgtcagaaattgtactgactcagtccccgggcacgctctcactcagtccaggcgaaagagctacgttgtcttgtcgcgcaagccagtccgtaagctctagcttcctcgcatggtatcaacaaaagcccgggcaggctccgcggctgctcatttactatgctagttcaagggctacgggaattccagaccgatttagtggatctgggagtggaactgacttcacacttacgatcagcaggcttgagccggaagattttgccgtgtactactgccagcaaactggaagaatccccccaacattcggacaaggcacgaaggtggaaatcaagattctggattatagctttggcggcggcgcgggccgcgatattccgccgccgctgattgaagaagcgtgcgaactgccggaatgccaggaagatgcgggcaacaaagtgtgcagcctgcagtgcaacaaccatgcgtgcggctgggatggcggcgattgcagcctgaactttaacgatccgtggaaaaactgcacccagagcctgcagtgctggaaatattttagcgatggccattgcgatagccagtgcaacagcgcgggctgcctgtttgatggctttgattgccagcgcgcggaaggccagtgcaacccgctgtatgatcagtattgcaaagatcattttagcgatggccattgcgatcagggctgcaacagcgcggaatgcgaatgggatggcctggattgcgcggaacatgtgccggaacgcctggcggcgggcaccctggtggtggtggtgctgatgccgccggaacagctgcgcaacagcagctttcattttctgcgcgaactgagccgcgtgctgcataccaacgtggtgtttaaacgcgatgcgcatggccagcagatgatttttccgtattatggccgcgaagaagaactgcgcaaacatccgattaaacgcgcggcggaaggctgggcggcgccggatgcgctgctgggccaggtgaaagcgagcctgctgccgggcggcagcgaaggcggccgccgccgccgcgaactggatccgatggatgtgcgcggcagcattgtgtatctggaaattgataaccgccagtgcgtgcaggcgagcagccagtgctttcagagcgcgaccgatgtggcggcgtttctgggcgcgctggcgagcctgggcagcctgaacattccgtataaaattgaagcggtgcagagcgaaaccgtggaaccgccgccgccggcgcagctgcattttatgtatgtggcggcggcggcgtttgtgctgctgttttttgtgggctgcggcgtgctgctgagccgcaaacgccgccgcatgaaactgctgagcagcattgaacaggcgtgcgatatttgccgcctgaaaaaactgaaatgcagcaaagaaaaaccgaaatgcgcgaaatgcctgaaaaacaactgggaatgccgctatagcccgaaaaccaaacgcagcccgctgacccgcgcgcatctgaccgaagtggaaagccgcctggaacgcctggaacagctgtttctgctgatttttccgcgcgaagatctggatatgattctgaaaatggatagcctgcaggatattaaagcgctgctgaccggcctgtttgtgcaggataacgtgaacaaagatgcggtgaccgatcgcctggcgagcgtggaaaccgatatgccgctgaccctgcgccagcatcgcattagcgcgaccagcagcagcgaagaaagcagcaacaaaggccagcgccagctgaccgtgagcgcggcggcgggcggcagcggcggcagcggcggcagcgatgcgctggatgattttgatctggatatgctgggcagcgatgcgctggatgattttgatctggatatgctgggcagcgatgcgctggatgattttgatctggatatgctgggcagcgatgcgctggatgattttgatctggatatgctg

SEQ ID NO: 123 - DNA, 339 bp, synthetic construct
  misc_feature 1..339: note = Description of Artificial Sequence: Synthetic polynucleotide
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 124 - DNA, 336 bp, synthetic construct
  misc_feature 1..336: note = Description of Artificial Sequence: Synthetic polynucleotide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 125 - DNA, 336 bp, synthetic construct; identical to SEQ ID NO: 124
  misc_feature 1..336: note = Description of Artificial Sequence: Synthetic polynucleotide
  source 1..336: mol_type = other DNA

SEQ ID NO: 126 - DNA, 336 bp, synthetic construct
  misc_feature 1..336: note = Description of Artificial Sequence: Synthetic polynucleotide
  source 1..336: mol_type = other DNA
cgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 127 - DNA, 108 bp, synthetic construct
  misc_feature 1..108: note = Description of Artificial Sequence: Synthetic polynucleotide
  source 1..108: mol_type = other DNA
cgcgatcagcgcctgccgccggatgcgcataaaccgccgggcggcggcagctttcgcaccccgattcaggaagaacaggcggatgcgcatagcaccctggcgaaaatt

SEQ ID NO: 128 - DNA, 57 bp, synthetic construct; identical to SEQ ID NO: 62
  misc_feature 1..57: note = Description of Artificial Sequence: Synthetic oligonucleotide
  source 1..57: mol_type = other DNA

SEQ ID NO: 129 - DNA, 708 bp, synthetic construct
  misc_feature 1..708: note = Description of Artificial Sequence: Synthetic polynucleotide
  source 1..708: mol_type = other DNA
gaagtgcagctgctggaaagcggcggcggcctggtgcagccgggcggcagcctgcgcctgagctgcgcggcgagcggctttacctttagcagctttagcatgagctgggtgcgccaggcgccgggcaaaggcctggaatgggtgagcagcattagcggcagcagcggcaccacctattatgcggatagcgtgaaaggccgctttaccattagccgcgataacagcaaaaacaccctgtatctgcagatgaacagcctgcgcgcggaagataccgcggtgtattattgcgcgaaaccgtttccgtattttgattattggggccagggcaccctggtgaccgtgagcagcggcgatggcagcagcggcggcagcggcggcgcgagcgaaattgtgctgacccagagcccgggcaccctgagcctgagcccgggcgaacgcgcgaccctgagctgccgcgcgagccagagcgtgagcagcagctttctggcgtggtatcagcagaaaccgggccaggcgccgcgcctgctgatttattatgcgagcagccgcgcgaccggcattccggatcgctttagcggcagcggcagcggcaccgattttaccctgaccattagccgcctggaaccggaagattttgcggtgtattattgccagcagaccggccgcattccgccgacctttggccagggcaccaaagtggaaattaaa